(19) United States
(12) Patent Application Publication
(10) Pub. No.: US 2020/0316221 A1
(43) Pub. Date: Oct. 8, 2020

(54) AAV CAPSID DESIGNS

(71) Applicant: University of Massachusetts, Boston, MA (US)

(72) Inventors: Guangping Gao, Westborough, MA (US); Guangchao Xu, Worcester, MA (US); Phillip Tai, Worcester, MA (US); Yuquan Wei, Chengdu, Sichuan (CN); Li Luo, Worcester, MA (US)

(73) Assignee: University of Massachusetts, Boston, MA (US)

(21) Appl. No.: 16/842,679

(22) Filed: Apr. 7, 2020

Related U.S. Application Data
(63) Continuation-in-part of application No. 16/341,504, filed on Apr. 12, 2019.
(60) Provisional application No. 62/486,642, filed on Apr. 18, 2017.
(60) Provisional application No. 62/417,756, filed on Nov. 4, 2016.
(60) Provisional application No. 62/408,022, filed on Oct. 13, 2016.
(60) Provisional application No. 63/003,143, filed on Mar. 31, 2020.

(51) Int. Cl. A61K 48/00; A61K 9/00; A61K 35/76

(52) U.S. Cl. CPC A61K 48/0008; A61K 35/76; A61K 9/0019; A61K 9/0085

(57) ABSTRACT

The disclosure in some aspects relates to recombinant adeno-associated viruses having distinct tissue targeting capabilities. In some aspects, the disclosure relates to gene transfer methods using the recombinant adeno-associated viruses. In some aspects, the disclosure relates to isolated AAV capsid proteins and isolated nucleic acids encoding the same.

### RELATED APPLICATIONS

This application is a continuation-in-part application of U.S. application Ser. No. 16/341,504, filed Apr. 12, 2019, entitled “AAV CAPSID DESIGNS”, which is a National Stage Application of PCT/US2017/056614, filed Oct. 13, 2017, entitled “AAV CAPSID DESIGNS”, which claims the benefit under 35 U.S.C. § 119(e) of the filing date of U.S. provisional application serial numbers U.S. Ser. No. 62/486,642, filed Apr. 18, 2017, entitled “AAV CAPSID DESIGNS”, 62/417,756, filed Nov. 4, 2016, entitled “AAV CAPSID DESIGNS”, and 62/408,022, filed Oct. 13, 2016, entitled “AAV CAPSID DESIGNS”; and claims the benefit under 35 U.S.C. § 119(e) of the filing date of U.S. provisional application Ser. No. 63/003,143, filed Mar. 31, 2020, entitled “CENTRAL NERVOUS SYSTEM TARGETING CAPSIDS”; the entire contents of each application which are incorporated herein by reference.

### FIELD OF THE DISCLOSURE

The disclosure in some aspects relates to isolated nucleic acids, compositions, and kits useful for identifying adeno-associated viruses in cells. In some aspects, the disclosure provides novel AAVs and methods of use thereof as well as related kits.

### BACKGROUND

Recombinant AAV adeno-associated viruses (rAAVs) are capable of driving stable and sustained transgene expression in target tissues without notable toxicity and host immunogenicity. Thus, rAAVs are promising delivery vehicles for long-term therapeutic gene expression. However, low transduction efficiency and restricted tissue tropisms by currently available rAAV vectors can limit their application as feasible and efficacious therapies. Additionally, faithful clinical translation of leading therapeutic AAV serotypes derived from non-human tissues is a concern. Accordingly, a need remains for new AAV vectors for gene delivery.

### SUMMARY

The disclosure in some aspects relates to novel AAVs for gene therapy applications. In some embodiments, AAVs described herein comprise amino acid variations in one or more capsid proteins that confer new or enhanced tissue tropism properties. According to some embodiments, variants of AAV2, AAV2/3 (e.g., AAV2/3 hybrid), and AAV8 have been identified and are disclosed herein that possess useful tissue targeting properties. For example, variants of AAV8 are provided that are useful for transducing cells, such as, human hepatocytes (e.g., present in liver tissue), central nervous system cells (CNS cells), and others. Variants of AAV2, AAV2/3 (e.g., AAV2/3 hybrid), and AAV8 are provided that, in some embodiments, are useful for targeting cells of the ocular tissue (e.g., the eye), gastrointestinal tract, respiratory system, breast tissue, pancreatic tissue, urinary tract tissue, uterine tissue, tissue associate with certain cancers (e.g., breast cancer, prostate cancer, etc.), and other tissues. In some embodiments, the variant AAVs described herein target tissue other than the tissue targeted by their corresponding wild-type AAVs.

The disclosure in some aspects provides an isolated nucleic acid comprising a sequence encoding a polypeptide selected from the group consisting of: SEQ ID NO: 1-409, 435-868, and 1726-1988, which encodes an AAV capsid protein. In some embodiments, a fragment of the isolated nucleic acid is provided. In certain embodiments, the fragment of the isolated nucleic acid does not encode a peptide that is identical to a sequence of any one of SEQ ID NOs: 869, 870, or 871.

In some aspects, the disclosure provides a nucleic acid comprising a sequence selected from the group consisting of SEQ ID NO: 410-434, 876-1718, and 1989-2251. In some embodiments, the nucleic acid encodes an AAV capsid protein, or a variant thereof and/or an AAV assembly-activating protein (AAP), or a variant thereof. In some embodiments, the AAP is in a different open reading frame of the nucleic acid than the AAV capsid protein. In some embodiments, the AAP is AAV2 AAP (AAP-2), or variant thereof.

The disclosure in some aspects provides an isolated AAV capsid protein comprising an amino acid sequence selected from the group consisting of: SEQ ID NOs: 1-409, 435-868, and 1726-1988. In some embodiments, the isolated AAV capsid protein comprises a sequence selected from: SEQ ID NOs: 1-409, 837-852 or 1726-1814, wherein an amino acid of the sequence that is not identical to a corresponding amino acid of the sequence set forth as SEQ ID NO: 869 is replaced with a conservative substitution.

In some aspects, the disclosure provides AAV2/3 hybrid capsid proteins. In some embodiments, the isolated AAV capsid protein comprises a sequence selected from: SEQ ID NOs: 435-628 and 1815-1988, wherein an amino acid of the sequence that is not identical to a corresponding amino acid of the sequence set forth as SEQ ID NO: 869 or 870 is replaced with a conservative substitution.

In some embodiments, the isolated AAV capsid protein comprises a sequence selected from: SEQ ID NOs: 629-836 or 853-868, wherein an amino acid of the sequence that is not identical to a corresponding amino acid of the sequence set forth as SEQ ID NO: 871 is replaced with a conservative substitution.

In certain aspects of the disclosure, a composition is provided that comprises any of the foregoing isolated AAV capsid proteins. In some embodiments, the composition further comprises a pharmaceutically acceptable carrier. In some embodiments a composition of one or more of the isolated AAV capsid proteins of the disclosure and a physiologically compatible carrier is provided.

In certain aspects of the disclosure, a recombinant AAV (rAAV) is provided that comprises any of the foregoing isolated AAV capsid proteins. In some embodiments, a composition comprising the rAAV is provided. In certain embodiments, the composition comprising the rAAV further comprises a pharmaceutically acceptable carrier. A recombinant AAV is also provided, wherein the recombinant AAV includes one or more of the isolated AAV capsid proteins of the disclosure.

In some aspects of the disclosure, a host cell is provided that contains a nucleic acid that comprises a coding sequence selected from the group consisting of: SEQ ID NO: 410-434, 876-1718 and 1989-2251, that is operably linked to a promoter. In some embodiments, a composition comprising the host cell and a sterile cell culture medium is provided. In some embodiments, a composition comprising the host cell and a cryopreservative is provided.

According to some aspects of the disclosure, a method for delivering a transgene to a subject is provided. In some embodiments, the method comprises administering any of the foregoing rAAVs to a subject, wherein the rAAV comprises at least one transgene, and wherein the rAAV infects cells of a target tissue of the subject. In some embodiments, subject is selected from a mouse, a rat, a rabbit, a dog, a cat, a sheep, a pig, and a non-human primate. In one embodiment, the subject is a human.

In some embodiments, the at least one transgene is a protein coding gene. In certain embodiments, the at least one transgene encodes a small interfering nucleic acid. In certain embodiments, the small interfering nucleic acid is a miRNA. In certain embodiments, the small interfering nucleic acid is a miRNA sponge or TuD RNA that inhibits the activity of at least one miRNA in the subject. In certain embodiments, the miRNA is expressed in a cell of the target tissue In certain embodiments, the target tissue is liver, central nervous system (CNS), ocular, gastrointestinal, respiratory, breast, pancreas, urinary tract, or uterine tissue.

In some embodiments, the transgene expresses a transcript that comprises at least one binding site for a miRNA, wherein the miRNA inhibits activity of the transgene, in a tissue other than the target tissue, by hybridizing to the binding site.

In certain embodiments, the rAAV is administered to the subject intravenously, transdermally, intraocularly, intrathecally, intracererbally, orally, intramuscularly, subcutaneously, intranasally, or by inhalation.

According to some aspects of the disclosure, a method for generating a somatic transgenic animal model is provided. In some embodiments, the method comprises administering any of the foregoing rAAVs to a non-human animal, wherein the rAAV comprises at least one transgene, and wherein the rAAV infects cells of a target tissue of the non-human animal.

In some embodiments, the transgene is at least one protein coding gene. In certain embodiments, the transgene encodes at least one small interfering nucleic acid. In some embodiments, the transgene encodes at least one reporter molecule. In certain embodiments, the small interfering nucleic acid is a miRNA. In certain embodiments, the small interfering nucleic acid is a miRNA sponge or TuD RNA that inhibits the activity of at least one miRNA in the animal. In certain embodiments, the miRNA is expressed in a cell of the target tissue.

In certain embodiments, the target tissue is liver, central nervous system (CNS), ocular, gastrointestinal, respiratory, breast, pancreas, urinary tract, or uterine tissue.

In some embodiments, the transgene expresses a transcript that comprises at least one binding site for a miRNA, wherein the miRNA inhibits activity of the transgene, in a tissue other than the target tissue, by hybridizing to the binding site.

According to some aspects of the disclosure, methods are provided for generating a somatic transgenic animal model that comprise administering any of the foregoing rAAVs to a non-human animal, wherein the rAAV comprises at least one transgene, wherein the transgene expresses a transcript that comprises at least one binding site for a miRNA, wherein the miRNA inhibits activity of the transgene, in a tissue other than a target tissue, by hybridizing to the binding site of the transcript.

In some embodiments, the transgene comprises a tissue specific promoter or inducible promoter. In certain embodiments, the tissue specific promoter is a liver-specific thyroxin binding globulin (TBG) promoter, an insulin promoter, a glucagon promoter, a somatostatin promoter, mucin-2 promoter, a pancreatic polypeptide (PPY) promoter, a synapsin-1 (Syn) promoter, a retinoschisin promoter, a K12 promoter, a CC10 promoter, a surfactant protein C (SP-C) promoter, a PRC1 promoter, a RRM2 promoter, uroplakin 2 (UPII) promoter, or a lactoferrin promoter.

In certain embodiments, the rAAV is administered to the animal intravenously, transdermally, intraocularly, intrathecally, orally, intramuscularly, subcutaneously, intranasally, or by inhalation. According to some aspects of the disclosure, a somatic transgenic animal model is provided that is produced by any of the foregoing methods.

In other aspects of the disclosure, a kit for producing a rAAV is provided. In some embodiments, the kit comprises a container housing an isolated nucleic acid having a sequence of any one of SEQ ID NO: 410-434, 876-1718, and 1989-2251. In some embodiments, the kit comprises a container housing an isolated nucleic acid encoding a polypeptide having a sequence of any one of SEQ ID NO: 1-409, 435-868, or 1726-1988. In some embodiments, the kit further comprises instructions for producing the rAAV. In some embodiments, the kit further comprises at least one container housing a recombinant AAV vector, wherein the recombinant AAV vector comprises a transgene.

In other aspects of the disclosure, a kit is provided that comprises a container housing a recombinant AAV having any of the foregoing isolated AAV capsid proteins. In some embodiments, the container of the kit is a syringe.

In other aspects, the disclosure relates to the use of AAV based vectors as vehicles for, delivery of genes, therapeutic, prophylactic, and research purposes as well as the development of somatic transgenic animal models.

In some aspects, the disclosure relates to AAV serotypes that have demonstrated distinct tissue/cell type tropism and can achieve stable somatic gene transfer in animal tissues at levels similar to those of adenoviral vectors (e.g., up to 100% in vivo tissue transduction depending upon target tissue and vector dose) in the absence of vector related toxicology. In other aspects, the disclosure relates to AAV serotypes having liver, central nervous system (CNS), ocular, gastrointestinal, respiratory, breast, pancreas, urinary tract, or uterine tissue-targeting capabilities. These tissues are associated with a broad spectrum of human diseases including neurological, metabolic, diabetic, ocular, respiratory, gastrointestinal, urinary tract, and reproductive diseases and certain cancers.

In some embodiments the rAAV includes at least one transgene. The transgene may be one which causes a pathological state. In some embodiments, the transgene encoding a protein that treats a pathological state.

In another aspect the novel AAVs of the disclosure may be used in a method for delivering a transgene to a subject. The method is performed by administering a rAAV of the disclosure to a subject, wherein the rAAV comprises at least one transgene. In some embodiments the rAAV targets a predetermined tissue of the subject.

In another aspect the AAVs of the disclosure may be used in a method for generating a somatic transgenic animal model. The method is performed by administering a rAAV of the disclosure to an animal, wherein the rAAV comprises at least one transgene, wherein the transgene causes a pathological state, and wherein the rAAV targets a predetermined tissue of the animal.

The transgene may express a number of genes including cancer related genes, pro-apoptotic genes and apoptosis-related genes. In some embodiments the transgene expresses a small interfering nucleic acid capable of inhibiting expression of a cancer related gene. In other embodiments the transgene expresses a small interfering nucleic acid capable of inhibiting expression of an apoptosis-related gene. The small interfering nucleic acid in other embodiments is a miRNA or shRNA. According to other embodiments the transgene expresses a toxin, optionally wherein the toxin is DTA. In other embodiments the transgene expresses a reporter gene that is optionally a reporter enzyme, such as Beta-Galactosidase or a Fluorescent protein, such as GFP or luciferase.

The transgene may express a miRNA. In other embodiments the transgene expresses a miRNA sponge, wherein miRNA sponge inhibits the activity of one or more miRNAs in the animal. The miRNA may be an endogenous miRNA or it may be expressed in a cell of a liver, central nervous system (CNS), ocular, gastrointestinal, respiratory, breast, pancreas, urinary tract, or uterine tissue, in some embodiments.

The rAAV may transduce many different types of tissue, such as neurons, squamous epithelial cells, renal proximal or distal convoluted tubular cells, mucosa gland cells, blood vessel endothelial cells, endometrial cells, retinal cells, or certain cancer cells (e.g., breast cancer cells, prostate cancer cells, etc.).

In some embodiments the rAAV is administered at a dose of 10¹⁰, 10¹¹, 10¹², 10¹³, 10¹⁴, or 10¹⁵ genome copies per subject. In some embodiments the rAAV is administered at a dose of 10¹⁰, 10¹¹, 10¹², 10¹³, or 10¹⁴ genome copies per kg. The rAAV may be administered by any route. For instance it may be administered intravenously (e.g., by portal vein injection) in some embodiments.

In some embodiments the transgene includes a tissue specific promoter such as a liver-specific thyroxin binding globulin (TBG) promoter, an insulin promoter, a glucagon promoter, a somatostatin promoter, mucin-2 promoter, a pancreatic polypeptide (PPY) promoter, a synapsin-1 (Syn) promoter, a retinoschisin promoter, a K12 promoter, a CC10 promoter, a surfactant protein C (SP-C) promoter, a PRC1 promoter, a RRM2 promoter, uroplakin 2 (UPII) promoter, or a lactoferrin promoter.

The somatic transgenic animal model may be a mammal, such as a mouse, a rat, a rabbit, a dog, a cat, a sheep, a pig, a non-human primate.

In some embodiments a putative therapeutic agent may be administered to the somatic transgenic animal model to determine the effect of the putative therapeutic agent on the pathological state in the animal.

In another aspect the disclosure is a somatic transgenic animal produced by the methods described herein.

A kit for producing a rAAV that generates a somatic transgenic animal having a pathological state in a predetermined tissue is provided according to another aspect of the disclosure. The kit includes at least one container housing a recombinant AAV vector, at least one container housing a rAAV packaging component, and instructions for constructing and packaging the recombinant AAV.

The rAAV packaging component may include a host cell expressing at least one rep gene and/or at least one cap gene. In some embodiments the host cell is a 293 cell. In other embodiments the host cell expresses at least one helper virus gene product that affects the production of rAAV containing the recombinant AAV vector. The at least one cap gene may encode a capsid protein from an AAV serotype that targets the predetermined tissue.

In other embodiments a rAAV packaging component includes a helper virus optionally wherein the helper virus is an adenovirus or a herpes virus.

The rAAV vector and components therein may include any of the elements described herein. For instance, in some embodiments the rAAV vector comprises a transgene, such as any of the transgenes described herein. In some embodiments the transgene expresses a miRNA inhibitor (e.g., a miRNA sponge or TuD RNA), wherein miRNA inhibitor inhibits the activity of one or more miRNAs in the somatic transgenic animal.

In some aspects, the disclosure provides a method for delivering a transgene to a target cell in a subject, the method comprising intracranially administering to the subject a recombinant adeno-associated virus (rAAV) comprising: an isolated nucleic acid comprising a transgene encoding one or more gene products of interest; and an adeno-associated acid (AAV) capsid protein having the sequence set forth in SEQ ID NO: 66.

In some embodiments, intracranial administration comprises intrahippocampal injection.

In some embodiments, a target cell is a central nervous system (CNS) cell. In some embodiments, a CNS cell is a neuron, oligodendrocyte, astrocyte, or microglial cell.

In some embodiments, a subject is a mammal. In some embodiments, a subject is a human. In some embodiments, a subject is characterized by production of anti-AAV2 antibodies. In some embodiments, administration of the rAAV does not result in a neutralizing immune response against the rAAV by the subject.

In some embodiments, an isolated nucleic acid comprises AAV inverted terminal repeats (ITRs) flanking the transgene. In some embodiments, the nucleic acid sequence encoding the one or more gene products is operably linked to a promoter. In some embodiments, the one or more gene products comprise a protein or an inhibitory nucleic acid.

In some aspects, the disclosure provides a method for delivering a transgene to a target cell in a subject, the method comprising intravenously administering to the subject a recombinant adeno-associated virus (rAAV) comprising: an isolated nucleic acid comprising a transgene encoding one or more gene products of interest; and an adeno-associated acid (AAV) capsid protein having the sequence set forth in SEQ ID NO: 66, wherein the administration results in the rAAV crossing the blood brain barrier (BBB) of the subject.

In some embodiments, a target cell is a central nervous system (CNS) cell. In some embodiments, a CNS cell is a neuron, oligodendrocyte, astrocyte, or microglial cell.

In some embodiments, a subject is a mammal. In some embodiments, a subject is a human. In some embodiments, a subject is characterized by production of anti-AAV2 antibodies. In some embodiments, administration of the rAAV does not result in a neutralizing immune response against the rAAV by the subject.

In some embodiments, an isolated nucleic acid comprises AAV inverted terminal repeats (ITRs) flanking the transgene. In some embodiments, the nucleic acid sequence encoding the one or more gene products is operably linked to a promoter. In some embodiments, the one or more gene products comprise a protein or an inhibitory nucleic acid.

In some embodiments, recombinant AAVs (rAAVs) comprising the capsid protein variants described herein (e.g., AAVv66, SEQ ID NO: 66) are more efficiently packaged (e.g., 2-fold, 3-fold, 4-fold, 5-fold, 10-fold, 20-fold, 30-fold, 50-fold, 100-fold, or more) than rAAVs having certain wild-type AAV capsid proteins (e.g., AAV2 capsid protein, SEQ ID NO: 869).

Each of the limitations of the disclosure can encompass various embodiments of the disclosure. It is, therefore, anticipated that each of the limitations of the disclosure involving any one element or combinations of elements can be included in each aspect of the disclosure. This disclosure is not limited in its application to the details of construction and the arrangement of components set forth in the following description or illustrated in the drawings. The disclosure is capable of other embodiments and of being practiced or of being carried out in various ways.

### BRIEF DESCRIPTION OF THE DRAWINGS

FIGS. 1A-1B show workflow schematics for the identification of AAV variants. FIG. 1A depicts high-throughput detection of novel AAV variants in selected human tissues. Proviral capsid sequences are amplified using high-cycle PCR, followed by low-cycle PCR to barcode the amplicon libraries for multiplexed single-molecule, real-time (SMRT) sequencing. FIG. 1B shows a summary of the pipeline for bioinformatics analysis of sequencing data.

FIGS. 2A-2D show data relating to in vivo detection of FFLuc transgene activity with different administrations of selected AAV8 variants. FIG. 2A shows luciferase activities of different AAV8 variants were evaluated at week 6 after IV (intravenous), IM (intramuscular), or IN (intranasal) injection. FIGS. 2B-2D data relating to evaluation of FFLuc activity for each variant, B2 (FIG. 2B), B3 (FIG. 2C), and B61 (FIG. 2D), compared to AAV8 (mean±SD, n=3, t test).

FIGS. 3A-3B show data relating to evaluation of FFLuc transgene activity delivered by the AAV8 variant B61 compared to AAV9 at day 21 after neonatal injection. Luciferase activities and genome copies of brain (FIG. 3A) and spinal cord (FIG. 3B) were detected (mean±SD, n=5, t test).

FIGS. 4A-4B show data relating to in vivo detection of FFLuc transgene activity after right hindlimb intramuscular (IM) injection of the AAV8 variant B44 compared to AAV8. FIG. 4A shows whole animal Luciferase expression of variant B44 was evaluated at week 6 after IM injection. FIG. 4B shows evaluation of muscle (RTA, right tibialis anterior; LTA, left tibialis anterior), liver, and heart. Luciferase activities (left bar graph) and relative ratios (right bar graph) for B44 compared to AAV8 (mean±SD, n=3).

FIG. 5 shows a phylogenic comparison of AAV8 variants (B2, B3, B61) to other AAV serotypes.

FIG. 6A shows a schematic depiction of a workflow for the in vivo characterization of novel AAV variants by high-throughput tropism screening.

FIG. 6B shows a schematic depiction of a workflow for the NHP characterization of novel AAV variants by high-throughput tropism screening.

FIG. 7 shows a scatter plot displaying the distribution of distinct AAV2 capsid variants (409 total) and AAV2/3 variants (194 total) harboring one or more single-amino-acid variants.

FIG. 8 shows diagrams of vector constructs used in the multiplexed screening of discovered capsid variants. Unique 6-bp barcodes were cloned into transgenes and packaged into candidate capsid variants.

FIG. 9 shows a schematic of an indexed transgene and high-throughput sequencing library design to assess capsid variant tropism profiling. The indexed and adapter cassette containing a 6-bp barcode (1° barcode) and a BstEII restriction site can be cloned into vector constructs using flanking BsrGI and SacI sites. Whole crude DNA from rAAV-treated tissues containing both host genome and vector genomes was cut with BstEII enzyme. The resulting 5′-overhang was used to specifically ligate to an adapter containing a second barcode, which allows for further multiplexed sequencing and streamlining; and a 5′-biotin modification, which can be used to select for adapter-containing fragments using magnetic bead enrichment. Enriched material can then undergo PCR amplification using primers specific to adapter and transgene sequences to produce libraries for high-throughput sequencing. SEQ ID NOs.: 1719-1725 are shown from top to bottom.

FIGS. 10A-10D show transduction spread of rAAV2 and rAAVv66 following intrahippocampal injection. FIG. 10A shows native EGFP expression following rAAV2-CB6-Egfp or rAAVv66-CB6-Egfp injection via unilateral intrahippocampal administration. Scale bars=700 μm. FIG. 10B shows quantification of EGFP-positive surface normalized to DAPI-positive surface. Data is presented as the mean±SD; n=3. ****P<0.0001. FIG. 10C shows coronal brain schematic depicting sub-anatomical regions of interest in both contralateral and ipsilateral hemispheres. Cornu ammonis (CA1, CA2, CA3, CA4), dentate gyrus (DG), corpus callosum (CC), and cortex (CTX). FIG. 10D shows high-magnification images of rAAVv66 transduced sub-anatomical regions. Scale bars=50 μm.

FIGS. 11A-11P show transduction of major cell types of the brain by rAAVv66. (FIGS. 11A, 11E, 11I, 11M) Coronal sections of rAAVv66-CB6-Egfp transduced mouse brains. IF-stained sections with antibodies against NEUN (FIG. 11A neurons), GFAP (FIG. 11E astrocytes), IBA1 (FIG. 11I microglia), or OLIG2 (FIG. 11M oligodendrocytes) indicate the distribution of cell types across the brain. Native EGFP expression that colocalize with IF staining indicate positively transduced cell types. Scale bars=700 μm. (FIGS. 11B, 11F, 11J, 11N) 3D rendering of sub-anatomical regions of single representative frames from dashed line rectangle boxes within coronal section views (top panels) with single-cell representations from fields defined by dashed lined square boxes (bottom three panels). Left panels, total area EGFP and cell marker IF stains; center panels, colocalized EGFP with total cell marker IF stains; right panels, colocalized EGFP and cell marker IF stains. Scale bars=50 μm (top panels), 5 μm (bottom three panels). (FIGS. 11C, 11G, 11K, 11O) Quantification of cell type-specific IF staining across indicated hippocampal regions (x axes), normalized to DAPI signal. (FIGS. 11D, 11H, 11L, 11P) Quantification of cell type-specific transduction across indicated regions, normalized to total cell-type IF and DAPI signal. Data is presented as the mean±SD; n=3. Cornu ammonis (CA1, CA2, CA3, CA4), dentate gyrus (DG), corpus callosum (CC), and cortex (CTX).

FIGS. 12A-12E show biophysical analyses of AAVv66. (FIGS. 12A-12B) Heatmap displays of differential scanning fluorimetry (DSF) analyses to query capsid protein unfolding (uncoating) and DNA accessibility (vector genome extrusion) at pHs 7, 6, 5, and 4. (FIGS. 12C-12E) Each defining amino acid residue of AAVv66 was converted to those of AAV2 by site-directed mutagenesis and examined for changes in (FIG. 12C) packaging yield, (FIG. 12D) capsid stability, and (FIG. 12E) genome release at pH 7. Values represent mean±SD. p values were determined by one-way ANOVA. *p<0.05, **p<0.01, ***p<0.001, ****p<0.0001. n³ 3.

FIGS. 13A-13E show cryo-EM primary metrics, map reconstruction, and model generation of AAVv66. (FIG. 13A) Density map of AAVv66. Color scheme demarcates the topological distance from the center (A). (FIG. 13B) Ribbon structure of the refined AAVv66 capsid monomer. Amino acids differentiating from AAV2 are highlighted. The 2-fold (oval), 3-fold (triangle), and 5-fold (pentagon) symmetries are annotated. Part of AAVv66 electron density (dark grey mesh) and residues are shown for regions close to (FIG. 13C) L583, R487, Y533, and K532, (FIG. 13D) S446, D499, and S501, and (FIG. 13E) N407-T414.

FIG. 14 shows structural differences between AAVv66 and AAV2. At the center is the AAVv66 60-mer structure (grey). Amino acid residues unique to AAVv66 are highlighted in green, while amino acid residues for a single monomer that are in common with AAV2 are colored. Atomic models showing residue side chains of select regions with substantial difference between AAVv66 and AAV2. The alignments were made with using monomers of AAV2 (11p3) and AAVv66, with modeled side chains from neighboring residues displayed in grey. Annotations for amino acids shown are indicated as those belonging to AAVv66, the position number, and then AAV2.

FIGS. 15A-15C show differential capsid surface electrostatics between AAV2 and AAVv66. (FIG. 15A) Surface positive and negative charges are displayed for AAV2 and AAVv66 60-mer, trimer (3-fold symmetry), and pentamers (exterior and interior of the 5-fold symmetry) structures. Black arrows at the AAV2 60-mer and trimer structures indicate the approximate positions of R585 and R588 at a single 3-fold protrusion. (FIG. 15B) Zoom-in of amino acid residues at 585-588 of AAV2 and AAVv66. (FIG. 15C) Bar graphs of the zeta potentials of purified vectors as measured by a zetasizer. Values represent mean±SD, n=3.

FIG. 16 shows amino acid sequence of the AAVs/AAVv66 capsid. Amino acid differences between AAV2 and AAVv66 are highlighted. Variable region (VR) residues are denoted by short bars. The aA domain is demarcated by the dotted bar, and residues forming the b-sheets are marked with black arrows. Start positions for VP1, VP2, and VP3 are marked by greater-than symbol (>). The PLA domain within VP1 is denoted by a bar. The AAV2 strand corresponds to SEQ ID NO: 869, and the AAVv66 strand corresponds to SEQ ID NO: 66.

FIG. 17 shows AAVv66 produces higher vector yields than AAV2. Crude lysate PCR assays were performed on media and cellular lysates of HEK239 cells subjected to triple-transfection of pAAV and packaging plasmids for AAV2 or AAVv66. Values represent mean genome copies±SD, n=3.

FIG. 18 shows AAVv66 lacks strong heparin binding. Heparin competition assay showing transduction efficiency of AAV2-CB6-FLuc and AAVv66-CB6-FLuc in HEK293 cells in the presence of increasing amounts of heparin (x-axis). Luminescence values were scaled to values obtained for wells lacking heparin and set to 1 (y-axis). Values represent mean±SD, n=3. **, p<0.01 by 2-way ANOVA.

FIG. 19 shows in vitro infection efficiencies of AAV2, AAV3b, and AAVv66 in HEK293 cells. Vectors were packaged with CB6-FLuc. Cells were lysed 48-hr post-infection to assess the infectivity of vectors via detection of luciferase activity (RLU, relative light units). Data is displayed in log-scale. Values represent mean±SD, ***p<0.0001 by one-way ANOVA, n=3.

FIGS. 20A-20D show intravenous administration of AAVv66 vector shows transduction of the liver. Systemic injection of AAVv66-CB6-Fluc resulted in the transduction of the liver. rAAV2-CB6-Fluc or AAVv66-CB6-Fluc (1.0E11 GC/mouse) was injected into mice by tail vein administration. (FIG. 20A) After 14 days, mice were injected with luciferin substrate intraperitoneally and imaged. Although quantification of whole-body live bioluminescence of luciferase activity did not reveal significant differences in transduction of the liver between AAVv66-CB6-Fluc and AAV2-CB6-Fluc, isolation of liver tissues and quantification of luciferase activity and detection of vector genome copy by qPCR showed that AAVv66 is a significantly weaker transducer of liver than AAV2. (FIG. 20B) Total flux of the abdomen in acquired images was recorded. Tissues were harvested and assayed for luciferase activity (FIG. 20C) and vector genome abundance by qPCR (FIG. 20D). Values represent mean±SD, n=3. *, p<0.05 by Student's t test.

FIGS. 21A-21D show intramuscular administration of AAVv66 vector shows transduction of muscle. Intramuscular injection of AAVv66 into the tibialis anterior resulted in very little difference in transduction capacity when compared with the transduction of AAV2. AAV2-CB6-FLuc or AAVv66-CB6-FLuc (4.0E10 GC/mouse) was injected into mice by intramuscular administration into one hindlimb (tibialis anterior). (FIG. 21A) After 14 days, mice were injected with luciferin substrate intraperitoneally and imaged. (FIG. 21B) Total flux of the injected hindlimb in acquired images was recorded. Tissues were harvested and assayed for luciferase activity (FIG. 21C) and vector genome abundance by qPCR (FIG. 21D). Values represent mean±SD, n=3. *, p<0.05 by Student's t test.

FIGS. 22A-22D show immunological characterization of AAVv66. Mice were intramuscularly administrated by AAV2-CB6-Egfp vector (1E11 GC/mouse). Four weeks after administration, sera were collected for testing neutralizing antibody (NAb) titers against AAV2 or AAVv66 infection. NAb50 values for AAV2 (FIG. 22A) and AAVv66 (FIG. 22B) are defined as the titer dilution that can block 50% of the total transduction achievable by the vector packaged with the LacZ reporter gene. Left, NAb table summaries of individual animals tested. Right, transduction efficiencies were plotted against various serum dilutions. Values represent mean±SD. Dashed lines indicate mean NAb50 serum titers. (FIG. 22C) After the four-week period, mice were intramuscularly administrated with AAV2-hA1AT or AAVv66-hA1AT (1E11 GC/mouse) on the contralateral hindlimb. Serum A1AT levels were measured by ELISA at weeks 5, 6, 7, and 8. Values represent mean±SD, n=3. n.s., not significant; *, p<0.05; **, p<0.01; and ***, p<0.001 by 2-way ANOVA on cross-sectional data points. (FIG. 22D) Rabbit anti-AAV serum cross-reactivity. Rabbit antisera raised against AAV serotypes was tested for NAb to AAVv66 versus the homologous AAV serotype to assess relative cross reactivity. Log 2 values represent highest antibody dilution to achieve 50% inhibition of transduction.

FIGS. 23A-23B show cryo-EM primary metrics, map reconstruction, and model generation of AAVv66. (FIG. 23A) Cryo-electron micrograph of AAVv66. The scale bar represents 100 Å. (FIG. 23B) Fourier shell correlation for even and odd particles (FSC_part) for AAVv66.

FIG. 24 shows RMSD (Å) statistics comparing AAVv66 to AAV2 or AAV3b. Summary of the total and regional RMSD (Å) between AAVv66 and AAV2 (1LP3) or AAV3b (3KIC) measured across all alpha-carbon pairs indicated (AAV2 numbering) calculated by the rms_cur function within PyMOL. Full capsid structures of AAV2, 3b, and AAVv66 were aligned through optimized fit within the cryo-EM density map of AAVv66. Using a custom script within PyMOL, the distance values (Å) between individual alpha-carbon pairs for either AAV2 (upper) or AAV3b (lower) were quantitatively transformed for representation as both color and radial thickness for the corresponding residues of AAVv66.

### DETAILED DESCRIPTION

Adeno-associated virus (AAV) is a small (˜26 nm) replication-defective, non-enveloped virus that generally depends on the presence of a second virus, such as adenovirus or herpes virus, for its growth in cells. AAV is not known to cause disease and induces a very mild immune response. AAV can infect both dividing and non-dividing cells and may incorporate its genome into that of the host cell. These features make AAV a very attractive candidate for creating viral vectors for gene therapy. Prototypical AAV vectors based on serotype 2 provided a proof-of-concept for non-toxic and stable gene transfer in murine and large animal models, but exhibited poor gene transfer efficiency in many major target tissues. The disclosure in some aspects seeks to overcome this shortcoming by providing novel AAVs having distinct tissue targeting capabilities for gene therapy and research applications.

In some aspects of the disclosure new AAV capsid proteins are provided that have distinct tissue targeting capabilities. In some embodiments, an AAV capsid protein is isolated from the tissue to which an AAV comprising the capsid protein targets. In some aspects, methods for delivering a transgene to a target tissue in a subject are provided. The transgene delivery methods may be used for gene therapy (e.g., to treat disease) or research (e.g., to create a somatic transgenic animal model) applications.

### Methods for Discovering AAVs

Much of the biology of AAV is influenced by its capsid. Consequently, methods for discovering novel AAVs have been largely focused on isolating DNA sequences for AAV capsids. A central feature of the adeno-associated virus (AAV) latent life cycle is persistence in the form of integrated and/or episomal genomes in a host cell. Methods used for isolating novel AAV include PCR based molecular rescue of latent AAV DNA genomes, infectious virus rescue of latent proviral genome from tissue DNAs in vitro in the presence of adenovirus helper function, and rescue of circular proviral genome from tissue DNAs by rolling-circle-linear amplification, mediated by an isothermal phage Phi-29 polymerase. All of these isolation methods take advantage of the latency of AAV proviral DNA genomes and focus on rescuing persistent viral genomic DNA.

In some aspects, the disclosure relates to the discovery that novel AAV variants with desirable tissue tropisms can be identified from in vivo tissues of a subject. Without wishing to be bound by any particular theory, the use of in vivo tissue exploits the natural reservoir of genomic diversity observed among viral genomic sequences isolated from both normal and tumor tissues of a subject. Thus in some embodiments, in vivo tissues act as natural incubators for viral (e.g., viral capsid protein) diversity through selective pressure and/or immune evasion.

In some aspects, the disclosure relates to the discovery that PCR products resulting from amplification of AAV DNA (e.g., AAV DNA isolated or extracted from a host cell or in vivo tissue of a subject) can be subjected to high-throughput single-molecule, real-time (SMRT) sequencing to identify novel capsid protein variants. As used herein, “single-molecule, real-time (SMRT) sequencing” refers to a parallelized single molecule sequencing method, for example as described by Roberts et al. (2013) Genome Biology 14:405, doi:10.1186/gb-2013-14-7-405. Without wishing to be bound by any particular theory, the use of SMRT sequencing removes the need to perform viral genome reconstruction and chimera prediction from aligned short-read fragments obtained from other conventional high-throughput genome sequencing methodologies.

Endogenous latent AAV genomes are transcriptionally active in mammalian cells (e.g., cells of nonhuman primate tissues such as liver, spleen and lymph nodes). Without wishing to be bound by theory, it is hypothesized that to maintain AAV persistence in host, low levels of transcription from AAV genes could be required and the resulting cap RNA could serve as more suitable and abundant substrates to retrieve functional cap sequences for vector development. Both rep and cap gene transcripts are detected with variable abundances by RNA detection methods (e.g., RT-PCR). The presence of cap gene transcripts and ability to generate cDNA of cap RNA through reverse transcription (RT) in vitro significantly increases abundance of templates for PCR-based rescue of novel cap sequences from tissues and enhances the sensitivity of novel AAV discovery.

Novel cap sequences may also be identified by transfecting cells with total cellular DNAs isolated from the tissues that harbor proviral AAV genomes at very low abundance, The cells may be further transfected with genes that provide helper virus function (e.g., adenovirus) to trigger and/or boost AAV gene transcription in the transfected cells. In some embodiments, novel cap sequences of the disclosure may be identified by isolating cap mRNA from the transfected cells, creating cDNA from the mRNA (e.g., by RT-PCR) and sequencing the cDNA.

### Isolated Capsid Proteins and Nucleic Acids Encoding the Same

AAVs isolated from mammals, particularly non-human primates, are useful for creating gene transfer vectors for clinical development and human gene therapy applications. The disclosure provides in some aspects novel AAVs that have been discovered in various in vivo tissues (e.g., liver, brain, gastric, respiratory, breast, pancreatic, rectal, prostate, urologic, and cervical tissues) using the methods disclosed herein. In some embodiments, the tissue(s) in which a novel AAV variant is discovered is a cancerous tissue (e.g., a tumor or a cancer cell). In some embodiments, nucleic acids encoding capsid proteins of these novel AAVs have been discovered in viral genomic DNA isolated from the human tissues. Examples of tissues in which novel AAV capsid proteins have been discovered are described in Table 1. Nucleic acid and protein sequences as well as other information regarding the AAVs are set forth in Tables 3-5 and 8, and in the sequence listing.

Isolated nucleic acids of the disclosure that encode AAV capsid proteins include any nucleic acid having a sequence as set forth in any one of SEQ ID NOs: 410-435, 876-1718, or 1989-2251, as well as any nucleic acid having a sequence with substantial homology thereto. In some embodiments, isolated nucleic acids of the disclosure include any nucleic acid having a sequence encoding a polypeptide having a sequence as set forth in any one of SEQ ID NOs: 1-409, 435-868, and 1726-1988. In some embodiments, the disclosure provides an isolated nucleic acid that has substantial homology with a nucleic acid having a sequence as set forth in any one of SEQ ID NOs: 410-435, 876-1718, and 1989-2251, but that does not encode a protein having an amino acid sequence as set forth in SEQ ID NOs: 869, 870, or 871.

In some embodiments, isolated AAV capsid proteins of the disclosure include any protein having an amino acid sequence as set forth in any one of SEQ ID NOs: 1-409, 837-852, or 1726-1814 as well as any protein having substantial homology thereto. In some embodiments, the disclosure provides an isolated capsid protein that has substantial homology with a protein having a sequence as set forth in any one of SEQ ID NOs 1-409, 837-852, or 1726-1814, but that does not have an amino acid sequence as set forth in SEQ ID NO: 869.

In some embodiments, isolated AAV capsid proteins of the disclosure include any protein having an amino acid sequence as set forth in any one of SEQ ID NOs: 435-628 or 1815-1988 as well as any protein having substantial homology thereto. In some embodiments, the disclosure provides an isolated capsid protein that has substantial homology with a protein having a sequence as set forth in any one of SEQ ID NOs 435-628 or 1815-1988, but that does not have an amino acid sequence as set forth in SEQ ID NO: 869 or 870.

In some embodiments, isolated AAV capsid proteins of the disclosure include any protein having an amino acid sequence as set forth in any one of SEQ ID NOs: 629-836 or 853-868 as well as any protein having substantial homology thereto. In some embodiments, the disclosure provides an isolated capsid protein that has substantial homology with a protein having a sequence as set forth in any one of SEQ ID NOs 629-836 or 853-868, but that does not have an amino acid sequence as set forth in SEQ ID NO: 871.

“Homology” refers to the percent identity between two polynucleotide or two polypeptide moieties. The term “substantial homology”, when referring to a nucleic acid, or fragment thereof, indicates that, when optimally aligned with appropriate nucleotide insertions or deletions with another nucleic acid (or its complementary strand), there is nucleotide sequence identity in about 90 to 100% of the aligned sequences. When referring to a polypeptide, or fragment thereof, the term “substantial homology” indicates that, when optimally aligned with appropriate gaps, insertions or deletions with another polypeptide, there is nucleotide sequence identity in about 90 to 100% of the aligned sequences. The term “highly conserved” means at least 80% identity, preferably at least 90% identity, and more preferably, over 97% identity. In some cases, highly conserved may refer to 100% identity. Identity is readily determined by one of skill in the art by, for example, the use of algorithms and computer programs known by those of skill in the art.

As described herein, alignments between sequences of nucleic acids or polypeptides are performed using any of a variety of publicly or commercially available Multiple Sequence Alignment Programs, such as “Clustal W”, accessible through Web Servers on the internet. Alternatively, Vector NTI utilities may also be used. There are also a number of algorithms known in the art that can be used to measure nucleotide sequence identity, including those contained in the programs described above. As another example, polynucleotide sequences can be compared using BLASTN, which provides alignments and percent sequence identity of the regions of the best overlap between the query and search sequences. Similar programs are available for the comparison of amino acid sequences, e.g., the “Clustal X” program, BLASTP. Typically, any of these programs are used at default settings, although one of skill in the art can alter these settings as needed. Alternatively, one of skill in the art can utilize another algorithm or computer program that provides at least the level of identity or alignment as that provided by the referenced algorithms and programs. Alignments may be used to identify corresponding amino acids between two proteins or peptides. A “corresponding amino acid” is an amino acid of a protein or peptide sequence that has been aligned with an amino acid of another protein or peptide sequence. Corresponding amino acids may be identical or non-identical. A corresponding amino acid that is a non-identical amino acid may be referred to as a variant amino acid. Table 6 provides examples of variant amino acids.

Alternatively for nucleic acids, homology can be determined by hybridization of polynucleotides under conditions that form stable duplexes between homologous regions, followed by digestion with single-stranded-specific nuclease(s), and size determination of the digested fragments. DNA sequences that are substantially homologous can be identified in a Southern hybridization experiment under, for example, stringent conditions, as defined for that particular system. Defining appropriate hybridization conditions is within the skill of the art.

A “nucleic acid” sequence refers to a DNA or RNA sequence. In some embodiments, the term nucleic acid captures sequences that include any of the known base analogues of DNA and RNA such as, but not limited to 4-acetylcytosine, 8-hydroxy-N6-methyladenosine, aziridinylcytosine, pseudoisocytosine, 5-(carboxyhydroxyl-methyl) uracil, 5-fluorouracil, 5-bromouracil, 5-carboxymethylaminomethyl-2-thiouracil, 5-carboxymethyl-aminomethyluracil, dihydrouracil, inosine, N6-isopentenyladenine, 1-methyladenine, 1-methylpseudo-uracil, 1-methylguanine, 1-methylinosine, 2,2-dimethyl-guanine, 2-methyladenine, 2-methylguanine, 3-methyl-cytosine, 5-methylcytosine, N6-methyladenine, 7-methylguanine, 5-methylaminomethyluracil, 5-methoxy-amino-methyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, -uracil-5-oxyacetic acid methylester, uracil-5-oxyacetic acid, pseudouracil, queosine, 2-thiocytosine, and 2,6-diaminopurine.

In some embodiments, proteins and nucleic acids of the disclosure are isolated. As used herein, the term “isolated” means artificially obtained or produced. As used herein with respect to nucleic acids, the term “isolated” generally means: (i) amplified in vitro by, for example, polymerase chain reaction (PCR); (ii) recombinantly produced by cloning; (iii) purified, as by cleavage and gel separation; or (iv) synthesized by, for example, chemical synthesis. An isolated nucleic acid is one that is readily manipulable by recombinant DNA techniques well known in the art. Thus, a nucleotide sequence contained in a vector in which 5′ and 3′ restriction sites are known or for which polymerase chain reaction (PCR) primer sequences have been disclosed is considered isolated but a nucleic acid sequence existing in its native state in its natural host is not. An isolated nucleic acid may be substantially purified, but need not be. For example, a nucleic acid that is isolated within a cloning or expression vector is not pure in that it may comprise only a tiny percentage of the material in the cell in which it resides. Such a nucleic acid is isolated, however, as the term is used herein because it is readily manipulable by standard techniques known to those of ordinary skill in the art. As used herein with respect to proteins or peptides, the term “isolated” generally refers to a protein or peptide that has been artificially obtained or produced (e.g., by chemical synthesis, by recombinant DNA technology, etc.).

It should be appreciated that conservative amino acid substitutions may be made to provide functionally equivalent variants, or homologs of the capsid proteins. In some aspects the disclosure embraces sequence alterations that result in conservative amino acid substitutions. As used herein, a conservative amino acid substitution refers to an amino acid substitution that does not alter the relative charge or size characteristics of the protein in which the amino acid substitution is made. Variants can be prepared according to methods for altering polypeptide sequence known to one of ordinary skill in the art such as are found in references that compile such methods, e.g., Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. Conservative substitutions of amino acids include substitutions made among amino acids within the following groups: (a) M, I, L, V; (b) F, Y, W; (c) K, R, H; (d) A, G; (e) S, T; (f) Q, N; and (g) E, D. Therefore, one can make conservative amino acid substitutions to the amino acid sequence of the proteins and polypeptides disclosed herein.

An example of an isolated nucleic acid that encodes a polypeptide comprising an AAV capsid protein is a nucleic acid having a sequence selected from the group consisting of: SEQ ID NO: 410-434, 876-1718, and 1989-2251. A fragment of an isolated nucleic acid encoding an AAV capsid sequence may be useful for constructing a nucleic acid encoding a desired capsid sequence. Fragments may be of any appropriate length. In some embodiments, a fragment (portion) of an isolated nucleic acid encoding an AAV capsid sequence may be useful for constructing a nucleic acid encoding a desired capsid sequence. Fragments may be of any appropriate length (e.g., at least 6, at least 9, at least 18, at least 36, at least 72, at least 144, at least 288, at least 576, at least 1152 or more nucleotides in length). For example, a fragment of nucleic acid sequence encoding a polypeptide of a first AAV capsid protein may be used to construct, or may be incorporated within, a nucleic acid sequence encoding a second AAV capsid sequence to alter the properties of the AAV capsid. In some embodiments, AAV capsid proteins that comprise capsid sequence fragments from multiple AAV serotypes are referred to as chimeric AAV capsids. The fragment may be a fragment that does not encode a peptide that is identical to a sequence of any one of SEQ ID NOs: 869, 870, or 871. For example, a fragment of nucleic acid sequence encoding a variant amino acid (compared with a known AAV serotype) may be used to construct, or may be incorporated within, a nucleic acid sequence encoding an AAV capsid sequence to alter the properties of the AAV capsid. In some embodiments, a nucleic acid sequence encoding an AAV variant may comprise about 1 to about 100 amino acid variants compared with a known AAV serotype (e.g., AAV serotype 2, AAV2/3 (e.g., AAV2/3 hybrid) or AAV8). In some embodiments, a nucleic acid sequence encoding an AAV variant may comprise about 5 to about 50 amino acid variants compared with a known AAV serotype (e.g., AAV serotype 2, AAV2/3 (e.g., AAV2/3 hybrid) or AAV8). In some embodiments, a nucleic acid sequence encoding an AAV variant may comprise about 10 to about 30 amino acid variants compared with a known AAV serotype (e.g., AAV serotype 2, AAV2/3 (e.g., AAV2/3 hybrid) or AAV8). In some embodiments, a nucleic acid sequence encoding an AAV variant may comprise 1, or 2, or 3, or 4, 5, or 6, or 7, or 8, or 9, or 10, or 11, or 12, or 13, or 14, or 15, or 16, or 17, or 18, or 19, or 20 amino acid variants compared with a known AAV serotype (e.g., AAV serotype 2, AAV2/3 (e.g., AAV2/3 hybrid) or AAV8). For example, a nucleic sequence encoding an AAV variant (e.g., SEQ ID NO: 861 may comprise 3 amino acid variants compared with a known AAV serotype (e.g., AAV8). A recombinant cap sequence may be constructed having one or more of the 3 amino acid variants by incorporating fragments of a nucleic acid sequence comprising a region encoding a variant amino acid into the sequence of a nucleic acid encoding the known AAV serotype. The fragments may be incorporated by any appropriate method, including using site directed mutagenesis. Thus, new AAV variants may be created having new properties.

In some aspects, the disclosure provides isolated nucleic acids encoding AAV assembly-activating proteins (AAPs), or variants thereof. As used herein, an “assembly activating protein” or “AAP” is a protein chaperone that functions to target newly synthesized capsid proteins (e.g., VP proteins, such as AAV VP1, VP2, and VP3) to the nucleolus of a cell thereby promoting encapsidation of viral genomes. Generally, an AAP is encoded in the cap gene of an adeno-associated virus. For example, AAP-2 is encoded in the cap gene of AAV2. Other examples of AAPs include but are not limited to AAP-1, AAP-3, AAP-4, AAP-5, AAP-8, AAP-9, AAP-11 and AAP-12, for example as described by Sonntag et al. J. Virol. 2011 December 85(23): 12686-12697. In some embodiments, an AAP is translated from a different open reading frame (ORF) of the cap gene than a capsid protein (e.g., VP1, VP2, VP3). For example, in some embodiments, a capsid protein (e.g., AAV2 VP1, VP2, VP3) is translated from ORF 1 of a cap gene and an AAP (e.g., AAP-2) is translated from ORF 2 of the cap gene. In some embodiments, an isolated nucleic acid encoding an AAP comprises or consists of a sequence selected from SEQ ID NO: 410-434 and 876-1718.

In some aspects, the disclosure relates to an AAVv66 capsid protein (e.g., an isolated nucleic acid encoding an AAVv66 capsid protein, a recombinant adeno-associated virus (rAAV) comprising an AAVv66 capsid protein, etc.), or a capsid protein having substantial homology to an AAVv66 capsid protein. In some embodiments, a capsid protein having substantial homology to an AAVv66 capsid protein is at least 50%, 60%, 70%, 80%, 90%, 95%, or 99% identical to the amino acid sequence set forth in SEQ ID NO: 66. In some embodiments, a capsid protein having substantial homology to an AAVv66 capsid protein comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acid substitutions, insertions, or deletions, relative to the amino acid sequence set forth in SEQ ID NO: 66. In some embodiments, a capsid protein having substantial homology to an AAVv66 capsid protein comprises more than 50 amino acid substitutions, insertions, or deletions, relative to the amino acid sequence set forth in SEQ ID NO: 66.

The disclosure relates, in some aspects, to the surprising discovery that rAAVs comprising AAVv66 capsid proteins are able to be produced in higher quantities in mammalian cell lines (e.g., HEK-293 cells) relative to rAAVs having certain other AAV capsid proteins (e.g., AAV2 capsid proteins, AAV3B capsid proteins, etc.). In some embodiments, transduced mammalian (e.g., HEK) producer cells yield between about 1.5-fold and about 5-fold (e.g., 1.5, 2, 3, 4, 5-fold) more rAAVs having AAVv66 capsid than mammalian (e.g., HEK) producer cells transduced with AAV2 capsid proteins. In some embodiments, transduced mammalian (e.g., HEK) producer cells yield between about 5% and about 50% (e.g., 5%, 10%, 15%, 20%, 25%, 30%, 35%, 40%, 45%, 50%, etc.) more rAAVs having AAVv66 capsid than mammalian (e.g., HEK) producer cells transduced with AAV3B capsid proteins.

Aspects of the disclosure relate to the unexpectedly improved central nervous system (CNS) cell transduction efficiency of AAVv66 capsid protein (e.g., rAAVs comprising AAVv66 capsid proteins) relative to rAAVs having AAV2 capsid proteins. In some embodiments, AAVv66-containing rAAVs transduce CNS cells at least 5%, 10%, 15%, 20%, 25%, 30%, 40%, 50%, 100%, 200%, 500%, 1000%, or more efficiently than AAV2-containing rAAVs. In some embodiments, the CNS cells are comprise neurons, oligodendrocytes, astrocytes, or microglial cells.

Aspects of the disclosure relate to certain AAV capsid proteins (e.g., AAVv66 capsid proteins) that are serologically distinct from other AAV capsid proteins (e.g., AAV1, AAV2, AAV3B, AAV8, AAV9, AAVrh.8, AAVrh.10, etc.). Without wishing to be bound by any particular theory, rAAVs comprising AAVv66 capsid proteins are not subject to the neutralizing antibody response in a subject that is sero-positive for antibodies against certain other AAV capsids. Accordingly, in some embodiments, rAAVs comprising AAVv66 capsid protein may be useful as a second-line therapy for delivery of transgenes to subjects that have previously been administered AAV therapies, or that are sero-positive for certain AAV capsid neutralizing antibodies.

In some aspects, the disclosure relates to rAAV capsid proteins (e.g., AAVv66 capsid protein) that exhibit increased thermostability relative to certain wild-type AAV capsid proteins (e.g., AAV2 capsid protein). In some embodiments, an AAVv66 capsid protein is more thermostable than an AAV2 capsid protein at a pH ranging from about pH 4 to about pH 7. In some embodiments, thermostability is determined by calculating the melting temperature of a capsid protein. In some embodiments, an AAVv66 capsid protein is characterized by a melting temperature that is between about 5° C. and about 10° C. above the melting temperature of an AAV2 capsid protein, at a given pH (e.g., between pH 4 and pH 7).

### Recombinant AAVs

In some aspects, the disclosure provides isolated AAVs. As used herein with respect to AAVs, the term “isolated” refers to an AAV that has been artificially obtained or produced. Isolated AAVs may be produced using recombinant methods. Such AAVs are referred to herein as “recombinant AAVs”. Recombinant AAVs (rAAVs) preferably have tissue-specific targeting capabilities, such that a transgene of the rAAV will be delivered specifically to one or more predetermined tissue(s). The AAV capsid is an important element in determining these tissue-specific targeting capabilities. Thus, an rAAV having a capsid appropriate for the tissue being targeted can be selected. In some embodiments, the rAAV comprises a capsid protein having an amino acid sequence as set forth in any one of SEQ ID NOs 1-409, 435-852, 859-874, or 1726-1988, or a protein having substantial homology thereto.

Methods for obtaining recombinant AAVs having a desired capsid protein are well known in the art. (See, for example, US 2003/0138772), the contents of which are incorporated herein by reference in their entirety). Typically the methods involve culturing a host cell which contains a nucleic acid sequence encoding an AAV capsid protein (e.g., a nucleic acid encoding a polypeptide having a sequence as set forth in any one of SEQ ID NOs 1-409, 435-868, or 1726-1988) or fragment thereof; a functional rep gene; a recombinant AAV vector composed of, AAV inverted terminal repeats (ITRs) and a transgene; and sufficient helper functions to permit packaging of the recombinant AAV vector into the AAV capsid proteins. In some embodiments, capsid proteins are structural proteins encoded by a cap gene of an AAV. In some embodiments, AAVs comprise three capsid proteins, virion proteins 1 to 3 (named VP1, VP2 and VP3), all of which may be expressed from a single cap gene. Accordingly, in some embodiments, the VP1, VP2 and VP3 proteins share a common core sequence. In some embodiments, the molecular weights of VP1, VP2 and VP3 are respectively about 87 kDa, about 72 kDa and about 62 kDa. In some embodiments, upon translation, capsid proteins form a spherical 60-mer protein shell around the viral genome. In some embodiments, the protein shell is primarily comprised of a VP3 capsid protein. In some embodiments, the functions of the capsid proteins are to protect the viral genome, deliver the genome and interact with the host. In some aspects, capsid proteins deliver the viral genome to a host in a tissue specific manner. In some embodiments, VP1 and/or VP2 capsid proteins may contribute to the tissue tropism of the packaged AAV. In some embodiments, the tissue tropism of the packaged AAV is determined by the VP3 capsid protein. In some embodiments, the tissue tropism of an AAV is enhanced or changed by mutations occurring in the capsid proteins.

In some aspects, the instant disclosure describes variants of wild-type AAV serotypes In some embodiments, the variants have altered tissue tropism. In some embodiments, the AAV variants described herein comprise amino acid variations (e.g., substitution, deletion, insertion) within the cap gene. As discussed above, all three capsid proteins are transcribed from a single cap gene. Accordingly, in some embodiments, an amino acid variation within a cap gene is present in all three capsid proteins encoded by said cap gene. Alternatively, in some embodiments, an amino acid variation may not be present in all three capsid proteins. In some embodiments, an amino acid variation occurs only in the VP1 capsid protein. In some embodiments, an amino acid variation occurs only in the VP2 capsid protein. In some embodiments, an amino acid variation occurs only within the VP3 capsid protein. In some embodiments, an AAV variant comprises more than one variation in a cap gene. In some embodiments, the more than one variation occur within the same capsid protein (e.g., within VP3). In some embodiments, the more than one variation occur within different capsid proteins (e.g., at least one variation in VP2 and at least one variation in VP3).

In some embodiments, the AAV variants described herein are variants of AAV2, AAV2/3 (e.g., AAV2/3 hybrid) or AAV8. AAV2 is known to efficiently transduce human central nervous system (CNS) tissue, kidney tissue, ocular tissue (e.g., photoreceptor cells and retinal pigment epithelium (RPE)), and other tissues. Accordingly, in some embodiments, the AAV3 variants described herein may be useful for delivering gene therapy to CNS tissue, kidney tissue, or ocular tissue. It is also known that AAV3 efficiently transduces cancerous human hepatocytes. Accordingly, in some embodiments, the AAV3 variants described herein may be useful for delivering gene therapy to cancerous and normal human hepatocytes. AAV8 is known to target tissue of the liver tissue, respiratory tissue, and the eye. Accordingly, in some embodiments, the AAV8 variants described herein may be useful for delivering gene therapy to the liver tissue, respiratory tissue or the eye.

It should be appreciated that the AAV2, AAV2/3 (e.g., AAV2/3 hybrid) and AAV8 variants described herein may comprise one or more variations within the cap gene compared with a corresponding wild-type AAV. Therefore, in some embodiments, the AAV2, AAV2/3 (e.g., AAV2/3 hybrid) and AAV8 variants described herein may have a tissue tropism useful for delivering gene therapy to additional tissue types that are not targeted by wild-type AAV2, AAV2/3 (e.g., AAV2/3 hybrid) or AAV8. For example, in some embodiments, AAV8 variants described herein (e.g., B61; SEQ ID NO: 865) may be useful for delivering gene therapy to the central nervous system (CNS). In some embodiments, AV2, AAV2/3 (e.g., AAV2/3 hybrid), or AAV8 variants described herein may be useful for targeting cells of the kidney or cells of the liver. In some embodiments, AAV2, AAV2/3 (e.g., AAV2/3 hybrid), or AAV8 variants described herein may be useful for targeting gene therapy to the liver, spleen, heart or brain.

In some aspects, AAV variants described herein may be useful for the treatment of CNS-related disorders. As used herein, a “CNS-related disorder” is a disease or condition of the central nervous system. A CNS-related disorder may affect the spinal cord (e.g., a myelopathy), brain (e.g., a encephalopathy) or tissues surrounding the brain and spinal cord. A CNS-related disorder may be of a genetic origin, either inherited or acquired through a somatic mutation. A CNS-related disorder may be a psychological condition or disorder, e.g., Attention Deficient Hyperactivity Disorder, Autism Spectrum Disorder, Mood Disorder, Schizophrenia, Depression, Rett Syndrome, etc. A CNS-related disorder may be an autoimmune disorder. A CNS-related disorder may also be a cancer of the CNS, e.g., brain cancer. A CNS-related disorder that is a cancer may be a primary cancer of the CNS, e.g., an astrocytoma, glioblastomas, etc., or may be a cancer that has metastasized to CNS tissue, e.g., a lung cancer that has metastasized to the brain. Further non-limiting examples of CNS-related disorders, include Parkinson's Disease, Lysosomal Storage Disease, Ischemia, Neuropathic Pain, Amyotrophic lateral sclerosis (ALS), Multiple Sclerosis (MS), and Canavan disease (CD).

In some embodiments, AAV variants described herein may be useful for delivering gene therapy to cardiac cells (e.g., heart tissue). Accordingly, in some embodiments, AAV variants described herein may be useful for the treatment of cardiovascular disorders. As used herein, a “cardiovascular disorder” is a disease or condition of the cardiovascular system. A cardiovascular disease may affect the heart, circulatory system, arteries, veins, blood vessels and/or capillaries. A cardiovascular disorder may be of a genetic origin, either inherited or acquired through a somatic mutation. Non-limiting examples of cardiovascular disorders include rheumatic heart disease, valvular heart disease, hypertensive heart disease, aneurysm, atherosclerosis, hypertension (e.g., high blood pressure), peripheral arterial disease (PAD), ischemic heart disease, angina, coronary heart disease, coronary artery disease, myocardial infarction, cerebral vascular disease, transient ischemic attack, inflammatory heart disease, cardiomyopathy, pericardial disease, congenital heart disease, heart failure, stroke, and myocarditis due to Chagas disease.

In some embodiments, AAV variants described herein may target the lung and/or tissue of the pulmonary system (e.g., respiratory system). Accordingly, in some embodiments, AAV variants described herein may be useful for treatment of pulmonary disease. As used herein a “pulmonary disease” is a disease or condition of the pulmonary system. A pulmonary disease may affect the lungs or muscles involved in breathing. A pulmonary disease may be of a genetic origin, either inherited or acquired through a somatic mutation. A pulmonary disease may be a cancer of the lung, including but not limited to, non-small cell lung cancer, small cell lung cancer, and lung carcinoid tumor. Further non-limiting examples of pulmonary diseases include acute bronchitis, acute respiratory distress syndrome (ARDS), asbestosis, asthma, bronchiectasis, bronchiolitis, bronchiolitis obliterans organizing pneumonia (BOOP), bronchopulmonary dysplasia, byssinosis, chronic bronchitis, coccidioidomycosis (Cocci), chronic obstructive pulmonary disorder (COPD), cryptogenic organizing pneumonia (COP), cystic fibrosis, emphysema, Hantavirus Pulmonary Syndrome, histoplasmosis, Human Metapneumovirus, hypersensitivity pneumonitis, influenza, lymphangiomatosis, mesothelioma, Middle Eastern Respiratory Syndrome, non-tuberculosis Mycobacterium, Pertussis, Pneumoconiosis (Black Lung Disease), pneumonia, primary ciliary dyskinesia, primary pulmonary hypertension, pulmonary arterial hypertension, pulmonary fibrosis, pulmonary vascular disease, Respiratory Syncytial Virus (RSV), sarcoidosis, Severe Acute Respiratory Syndrome (SARS), silicosis, sleep apnea, Sudden Infant Death Syndrome (SIDS), and tuberculosis.

In some embodiments, AAV variants described herein may target liver tissue. Accordingly, in some embodiments, AAV variants described herein may be useful for treatment of hepatic disease. As used herein a “hepatic disease” is a disease or condition of the liver. A hepatic disease may be of a genetic origin, either inherited or acquired through a somatic mutation. A hepatic disease may be a cancer of the liver, including but not limited to hepatocellular carcinoma (HCC), fibrolamellar carcinoma, cholangiocarcinoma, angiosarcoma and hepatoblastoma. Further non-limiting examples of pulmonary diseases include Alagille Syndrome, Alpha 1 Anti-Trypsin Deficiency, autoimmune hepatitis, biliary atresia, cirrhosis, cystic disease of the liver, fatty liver disease, galactosemia, gallstones, Gilbert's Syndrome, hemochromatosis, liver disease in pregnancy, neonatal hepatitis, primary biliary cirrhosis, primary sclerosing cholangitis, porphyria, Reye's Syndrome, sarcoidosis, toxic hepatitis, Type 1 Glycogen Storage Disease, tyrosinemia, viral hepatitis A, B, C, Wilson Disease, and schistosomiasis.

In some embodiments, AAV variants described herein may target kidney tissue. Accordingly, in some embodiments, AAV variants described herein may be useful for treatment of kidney disease. As used herein a “kidney disease” is a disease or condition of the liver. A kidney disease may be of a genetic origin, either inherited or acquired through a somatic mutation. A kidney disease may be a cancer of the kidney, including but not limited to renal cell cancer, clear cell cancer, papillary cancer type 1, papillary cancer type 2, chromophobe cancer, oncocytic cell cancer, collecting duct cancer, transitional cell cancer of the renal pelvis and Wilm's tumor. Further non-limiting examples of kidney disease include Abderhalden-Kaufmann-Lignac syndrome (Nephropathic Cystinosis), Acute Kidney Failure/Acute Kidney Injury, Acute Lobar Nephronia, Acute Phosphate Nephropathy, Acute Tubular Necrosis, Adenine Phosphoribosyltransferase Deficiency, Adenovirus Nephritis, Alport Syndrome, Amyloidosis, Angiomyolipoma, Analgesic Nephropathy, Angiotensin Antibodies and Focal Segmental Glomerulosclerosis, Antiphospholipid Syndrome, Anti-TNF-α Therapy-related Glomerulonephritis, APOL1 Mutations, Apparent Mneralocorticoid Excess Syndrome, Aristolochic Acid Nephropathy, Balkan Endemic Nephropathy, Bartter Syndrome, Beeturia, β-Thalassemia Renal Disease, Bile Cast Nephropathy, BK Polyoma, C1q Nephropathy, Cardiorenal syndrome, CFHR5 nephropathy, Cholesterol Emboli, Churg-Strauss syndrome, Chyluria, Collapsing Glomerulopathy, Collapsing Glomerulopathy Related to CMV, Congenital Nephrotic Syndrome, Conorenal syndrome (Mainzer-Saldino Syndrome or Saldino-Mainzer Disease), Contrast Nephropathy, Copper Sulfate Intoxication, Cortical Necrosis, Cryoglobuinemia, Crystal-Induced Acute Kidney injury, Cystic Kidney Disease, Acquired, Cystinuria, Dense Deposit Disease (MPGN Type 2), Dent Disease (X-linked Recessive Nephrolithiasis), Dialysis Disequilibrium Syndrome, Diabetic Kidney Disease, Diabetes Insipidus, EAST syndrome, Ectopic Ureter, Edema, Erdheim-Chester Disease, Fabry's Disease, Familial Hypocalciuric Hypercalcemia, Fanconi Syndrome, Fraser syndrome, Fibronectin Glomerulopathy, Fibrillary Glomerulonephritis and Immunotactoid Glomerulopathy, Fraley syndrome, Focal Segmental Glomerulosclerosis, Focal Sclerosis, Focal Glomerulosclerosis, Galloway Mowat syndrome, Gitelman Syndrome, Glomerular Diseases, Glomerular Tubular Reflux, Glycosuria, Goodpasture Syndrome, Hemolytic Uremic Syndrome (HUS), Atypical Hemolytic Uremic Syndrome (aHUS), Hemophagocytic Syndrome, Hemorrhagic Cystitis, Hemosiderosis related to Paroxysmal Nocturnal Hemoglobinuria and Hemolytic Anemia, Hepatic Veno-Occlusive Disease, Sinusoidal Obstruction Syndrome, Hepatitis C-Associated Renal Disease, Hepatorenal Syndrome, HIV-Associated Nephropathy (HIVAN), Horseshoe Kidney (Renal Fusion), Hunner's Ulcer, Hyperaldosteronism, Hypercalcemia, Hyperkalemia, Hypermagnesemia, Hypernatremia, Hyperoxaluria, Hyperphosphatemia, Hypocalcemia, Hypokalemia, Hypokalemia-induced renal dysfunction, Hypomagnesemia, Hyponatremia, Hypophosphatemia, IgA Nephropathy, IgG4 Nephropathy, Interstitial Cystitis, Painful Bladder Syndrome, Interstitial Nephritis, Ivemark's syndrome, Kidney Stones, Nephrolithiasis, Leptospirosis Renal Disease, Light Chain Deposition Disease, Monoclonal Immunoglobulin Deposition Disease, Liddle Syndrome, Lightwood-Albright Syndrome, Lipoprotein Glomerulopathy, Lithium Nephrotoxicity, LMX1B Mutations Cause Hereditary FSGS, Loin Pain Hematuria, Lupus, Systemic Lupus Erythematosis, Lupus Kidney Disease, Lupus Nephritis, Lyme Disease-Associated Glomerulonephritis, Malarial Nephropathy, Malignant Hypertension, Malakoplakia, Meatal Stenosis, Medullary Cystic Kidney Disease, Medullary Sponge Kidney, Megaureter, Melamine Toxicity and the Kidney, Membranoproliferative Glomerulonephritis, Membranous Nephropathy, MesoAmerican Nephropathy, Metabolic Acidosis, Metabolic Alkalosis, Microscopic Polyangiitis, Milk-alkalai syndrome, Minimal Change Disease, Multicystic dysplastic kidney, Multiple Myeloma, Myeloproliferative Neoplasms and Glomerulopathy, Nail-patella Syndrome, Nephrocalcinosis, Nephrogenic Systemic Fibrosis, Nephroptosis (Floating Kidney, Renal Ptosis), Nephrotic Syndrome, Neurogenic Bladder, Nodular Glomerulosclerosis, Non-Gonococcal, Nutcracker syndrome, Orofaciodigital Syndrome, Orthostatic Hypotension, Orthostatic Proteinuria, Osmotic Diuresis, Page Kidney, Papillary Necrosis, Papillorenal Syndrome (Renal-Coloboma Syndrome, Isolated Renal Hypoplasia), The Peritoneal-Renal Syndrome, Posterior Urethral Valve, Post-infectious Glomerulonephritis, Post-streptococcal Glomerulonephritis, Polyarteritis Nodosa, Polycystic Kidney Disease, Posterior Urethral Valves, Preeclampsia, Proliferative Glomerulonephritis with Monoclonal IgG Deposits (Nasr Disease), Proteinuria (Protein in Urine), Pseudohyperaldosteronism, Pseudohypoparathyroidism, Pulmonary-Renal Syndrome, Pyelonephritis (Kidney Infection), Pyonephrosis, Radiation Nephropathy, Refeeding syndrome, Reflux Nephropathy, Rapidly Progressive Glomerulonephritis, Renal Abscess, Peripnephric Abscess, Renal Agenesis, Renal Artery Aneurysm, Renal Artery Stenosis, Renal Cell Cancer, Renal Cyst, Renal Hypouricemia with Exercise-induced Acute Renal Failure, Renal Infarction, Renal Osteodystrophy, Renal Tubular Acidosis, Reset Osmostat, Retrocaval Ureter, Retroperitoneal Fibrosis, Rhabdomyolysis, Rhabdomyolysis related to Bariatric Sugery, Rheumatoid Arthritis-Associated Renal Disease, Sarcoidosis Renal Disease, Salt Wasting, Renal and Cerebral, Schimke immuno-osseous dysplasia, Scleroderma Renal Crisis, Serpentine Fibula-Polycystic Kidney Syndrome, Exner Syndrome, Sickle Cell Nephropathy, Silica Exposure and Chronic Kidney Disease, Kidney Disease Following Hematopoietic Cell Transplantation, Kidney Disease Related to Stem Cell Transplantation, Thin Basement Membrane Disease, Benign Familial Hematuria, Trigonitis, Tuberous Sclerosis, Tubular Dysgenesis, Tumor Lysis Syndrome, Uremia, Uremic Optic Neuropathy, Ureterocele, Urethral Caruncle, Urethral Stricture, Urinary Incontinence, Urinary Tract Infection, Urinary Tract Obstruction, Vesicointestinal Fistula, Vesicoureteral Reflux, Von Hippel-Lindau Disease, Warfarin-Related Nephropathy, Wegener's Granulomatosis, Granulomatosis with Polyangiitis, and Wunderlich syndrome.

In some embodiments, AAV variants described herein may be useful for delivering gene therapy to ocular tissue (e.g., tissue or cells of the eye). Accordingly, in some embodiments, AAV variants described herein may be useful for the treatment of ocular disorders. As used herein, an “ocular disorder” is a disease or condition of the eye. An ocular disease may affect the eye, sclera, cornea, anterior chamber, posterior chamber, iris, pupil, lens, vitreous humor, retina, or optic nerve. An ocular disorder may be of a genetic origin, either inherited or acquired through a somatic mutation. Non-limiting examples of ocular diseases and disorders include but are not limited to: age-related macular degeneration, retinopathy, diabetic retinopathy, macular edema, glaucoma, retinitis pigmentosa and eye cancer.

In some embodiments, AAV variants described herein may be useful for delivering gene therapy to gastrointestinal tissue (e.g., tissue of the gastrointestinal tract). Accordingly, in some embodiments, AAV variants described herein may be useful for the treatment of gastrointestinal tract disorders. As used herein, a “gastrointestinal tract disorder” is a disease or condition of the gastrointestinal tract. A gastrointestinal disease may affect the mucosa (e.g., epithelium, lamina propria, muscularis mucosae, etc.), submucosa (e.g., submucous plexus, enteric nervous plexis, etc.), muscular layer of the gastrointestinal tract, the serosa and/or adventitia, oral cavity, esophagus, pylorus, stomach duodenum, small intestine, caecum, appendix, colon, anal canal, or rectum. A gastrointestinal tract disorder may be of a genetic origin, either inherited or acquired through a somatic mutation. Non-limiting examples of gastrointestinal tract diseases and disorders include but are not limited to: inflammatory bowel disease (IBD), Crohn's disease, ulcerative colitis, irritable bowel syndrome, Celiac disease, gastroesophageal reflux disease (GERD), achakasua, diverticulitus, diarrhea, and certain cancers (e.g., bowel cancer, stomach cancer, colon cancer, rectal cancer, etc.).

In some embodiments, AAV variants described herein may be useful for delivering gene therapy to breast tissue (e.g., tissue of the breast). Accordingly, in some embodiments, AAV variants described herein may be useful for the treatment of breast disorders. As used herein, a “breast disorder” is a disease or condition of the breast. A breast disease may affect the fibrous tissue, fatty tissue, lobules, or ducts of the breast. A breast disorder may be of a genetic origin, either inherited or acquired through a somatic mutation. Non-limiting examples of breast diseases and disorders include but are not limited to: mastitis, breast calcification, fat necrosis, fibroadenoma, fibrosis and simple cysts, galactorrhea, hyperplasia and breast cancer.

In some embodiments, AAV variants described herein may be useful for delivering gene therapy to pancreatic tissue (e.g., tissue of the pancreas). Accordingly, in some embodiments, AAV variants described herein may be useful for the treatment of pancreatic disorders. As used herein, a “pancreatic disorder” is a disease or condition of the pancreas. A pancreatic disease may affect the head of the pancreas, neck of the pancreas, body of the pancreas, tail of the pancreas, pancreatic islets (e.g., islets of Langerhans), acini, or columnar epithelium. A pancreatic disorder may be of a genetic origin, either inherited or acquired through a somatic mutation. Non-limiting examples of pancreatic diseases and disorders include but are not limited to: diabetes (e.g., diabetes mellitus type 1 and diabetes mellitus type 2), pancreatitis (e.g., acute pancreatitis, chronic pancreatitis), and pancreatic cancer.

In some embodiments, AAV variants described herein may be useful for delivering gene therapy to urinary tract tissue (e.g., tissue of the urinary tract, such as bladder tissue). Accordingly, in some embodiments, AAV variants described herein may be useful for the treatment of urinary tract disorders. As used herein, a “urinary tract disorder” is a disease or condition of the urinary tract. A urinary tract disease may affect the bladder, ureters, urethera, or prostate. A urinary tract disorder may be of a genetic origin, either inherited or acquired through a somatic mutation. Non-limiting examples of urinary tract diseases and disorders include but are not limited to: urinary tract infections, kidney stones, bladder control problems (e.g., urinary retention, urinary incontinence, etc.), cystitis, and bladder cancer.

In some embodiments, AAV variants described herein may be useful for delivering gene therapy to uterine tissue (e.g., tissue of the uterus). Accordingly, in some embodiments, AAV variants described herein may be useful for the treatment of uterine disorders. As used herein, a “uterine disorder” is a disease or condition of the uterus. A uterine disease may affect the cervix, cervical canal, body of the uterus (fundus), endometrium, myometrium, or perimetrium. A uterine disorder may be of a genetic origin, either inherited or acquired through a somatic mutation. Non-limiting examples of uterine diseases and disorders include but are not limited to: adenomyosis, endometriosis, endometrial hyperplasia, Asherman's syndrome, and endometrial cancer.

The components to be cultured in the host cell to package a rAAV vector in an AAV capsid may be provided to the host cell in trans. Alternatively, any one or more of the required components (e.g., recombinant AAV vector, rep sequences, cap sequences, and/or helper functions) may be provided by a stable host cell which has been engineered to contain one or more of the required components using methods known to those of skill in the art. Most suitably, such a stable host cell will contain the required component(s) under the control of an inducible promoter. However, the required component(s) may be under the control of a constitutive promoter. Examples of suitable inducible and constitutive promoters are provided herein, in the discussion of regulatory elements suitable for use with the transgene. In still another alternative, a selected stable host cell may contain selected component(s) under the control of a constitutive promoter and other selected component(s) under the control of one or more inducible promoters. For example, a stable host cell may be generated which is derived from 293 cells (which contain E1 helper functions under the control of a constitutive promoter), but which contain the rep and/or cap proteins under the control of inducible promoters. Still other stable host cells may be generated by one of skill in the art.

The recombinant AAV vector, rep sequences, cap sequences, and helper functions required for producing the rAAV of the disclosure may be delivered to the packaging host cell using any appropriate genetic element (vector). In some embodiments, a single nucleic acid encoding all three capsid proteins (e.g., VP1, VP2 and VP3) is delivered into the packaging host cell in a single vector. In some embodiments, nucleic acids encoding the capsid proteins are delivered into the packaging host cell by two vectors; a first vector comprising a first nucleic acid encoding two capsid proteins (e.g., VP1 and VP2) and a second vector comprising a second nucleic acid encoding a single capsid protein (e.g., VP3). In some embodiments, three vectors, each comprising a nucleic acid encoding a different capsid protein, are delivered to the packaging host cell. The selected genetic element may be delivered by any suitable method, including those described herein. The methods used to construct any embodiment of this disclosure are known to those with skill in nucleic acid manipulation and include genetic engineering, recombinant engineering, and synthetic techniques. See, e.g., Sambrook et al, Molecular Cloning: A Laboratory Manual, Cold Spring Harbor Press, Cold Spring Harbor, N.Y. Similarly, methods of generating rAAV virions are well known and the selection of a suitable method is not a limitation on the present disclosure. See, e.g., K. Fisher et al, J. Virol., 70:520-532 (1993) and U.S. Pat. No. 5,478,745.

In some embodiments, recombinant AAVs may be produced using the triple transfection method (described in detail in U.S. Pat. No. 6,001,650). Typically, the recombinant AAVs are produced by transfecting a host cell with a recombinant AAV vector (comprising a transgene) to be packaged into AAV particles, an AAV helper function vector, and an accessory function vector. An AAV helper function vector encodes the “AAV helper function” sequences (e.g., rep and cap), which function in trans for productive AAV replication and encapsidation. Preferably, the AAV helper function vector supports efficient AAV vector production without generating any detectable wild-type AAV virions (e.g., AAV virions containing functional rep and cap genes). Non-limiting examples of vectors suitable for use with the present disclosure include pHLP19, described in U.S. Pat. No. 6,001,650 and pRep6cap6 vector, described in U.S. Pat. No. 6,156,303, the entirety of both incorporated by reference herein. The accessory function vector encodes nucleotide sequences for non-AAV derived viral and/or cellular functions upon which AAV is dependent for replication (e.g., “accessory functions”). The accessory functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the known helper viruses such as adenovirus, herpesvirus (other than herpes simplex virus type-1), and vaccinia virus.

In some aspects, the disclosure provides transfected host cells. The term “transfection” is used to refer to the uptake of foreign DNA by a cell, and a cell has been “transfected” when exogenous DNA has been introduced inside the cell (e.g., across the cell membrane). A number of transfection techniques are generally known in the art. See, e.g., Graham et al. (1973) Virology, 52:456, Sambrook et al. (1989) Molecular Cloning, a laboratory manual, Cold Spring Harbor Laboratories, New York, Davis et al. (1986) Basic Methods in Molecular Biology, Elsevier, and Chu et al. (1981) Gene 13:197. Such techniques can be used to introduce one or more exogenous nucleic acids, such as a nucleotide integration vector and other nucleic acid molecules, into suitable host cells.

A “host cell” refers to any cell that harbors, or is capable of harboring, a substance of interest. Often a host cell is a mammalian cell. A host cell may be used as a recipient of an AAV helper construct, an AAV minigene plasmid, an accessory function vector, or other transfer DNA associated with the production of recombinant AAVs. The term includes the progeny of the original cell that has been transfected. Thus, a “host cell” as used herein may refer to a cell that has been transfected with an exogenous DNA sequence. It is understood that the progeny of a single parental cell may not necessarily be completely identical in morphology or in genomic or total DNA complement as the original parent, due to natural, accidental, or deliberate mutation.

As used herein, the term “cell line” refers to a population of cells capable of continuous or prolonged growth and division in vitro. Often, cell lines are clonal populations derived from a single progenitor cell. It is further known in the art that spontaneous or induced changes can occur in karyotype during storage or transfer of such clonal populations. Therefore, cells derived from the cell line referred to may not be precisely identical to the ancestral cells or cultures, and the cell line referred to includes such variants.

As used herein, the terms “recombinant cell” refers to a cell into which an exogenous DNA segment, such as DNA segment that leads to the transcription of a biologically-active polypeptide or production of a biologically active nucleic acid such as an RNA, has been introduced.

Cells may also be transfected with a vector (e.g., helper vector) that provides helper functions to the AAV. The vector providing helper functions may provide adenovirus functions, including, e.g., E1a, E1b, E2a, and E4ORF6. The sequences of adenovirus gene providing these functions may be obtained from any known adenovirus serotype, such as serotypes 2, 3, 4, 7, 12 and 40, and further including any of the presently identified human types known in the art. Thus, in some embodiments, the methods involve transfecting the cell with a vector expressing one or more genes necessary for AAV replication, AAV gene transcription, and/or AAV packaging.

As used herein, the term “vector” includes any genetic element, such as a plasmid, phage, transposon, cosmid, chromosome, artificial chromosome, virus, virion, etc., that is capable of replication when associated with the proper control elements and which can transfer gene sequences between cells. Thus, the term includes cloning and expression vehicles, as well as viral vectors. In some embodiments, useful vectors are contemplated to be those vectors in which the nucleic acid segment (e.g., nucleic acid sequence) to be transcribed is positioned under the transcriptional control of a promoter. A “promoter” refers to a DNA sequence recognized by the synthetic machinery of the cell, or introduced synthetic machinery, that is required to initiate the specific transcription of a gene. The phrases “operatively positioned,” “under control” or “under transcriptional control” means that the promoter is in the correct location and orientation in relation to the nucleic acid to control RNA polymerase initiation and expression of the gene. The term “expression vector or construct” means any type of genetic construct containing a nucleic acid in which part or all of the nucleic acid encoding sequence is capable of being transcribed. In some embodiments, expression includes transcription of the nucleic acid, for example, to generate a biologically-active polypeptide product or inhibitory RNA (e.g., shRNA, miRNA, miRNA inhibitor) from a transcribed gene.

In some cases, an isolated capsid gene can be used to construct and package recombinant AAVs, using methods well known in the art, to determine functional characteristics associated with the capsid protein encoded by the gene. For example, isolated capsid genes can be used to construct and package a recombinant AAV (rAAV) comprising a reporter gene (e.g., B-Galactosidase, GFP, Luciferase, etc.). The rAAV can then be delivered to an animal (e.g., mouse) and the tissue targeting properties of the novel isolated capsid gene can be determined by examining the expression of the reporter gene in various tissues (e.g., heart, liver, kidneys) of the animal. Other methods for characterizing the novel isolated capsid genes are disclosed herein and still others are well known in the art.

The foregoing methods for packaging recombinant vectors in desired AAV capsids to produce the rAAVs of the disclosure are not meant to be limiting and other suitable methods will be apparent to the skilled artisan.

Recombinant AAV vectors.

“Recombinant AAV (rAAV) vectors” of the disclosure are typically composed of, at a minimum, a transgene and its regulatory sequences, and 5′ and 3′ AAV inverted terminal repeats (ITRs). It is this recombinant AAV vector which is packaged into a capsid protein and delivered to a selected target cell. In some embodiments, the transgene is a nucleic acid sequence, heterologous to the vector sequences, that encodes a polypeptide, protein, functional RNA molecule (e.g., miRNA, miRNA inhibitor) or other gene product, of interest. The nucleic acid coding sequence is operatively linked to regulatory components in a manner that permits transgene transcription, translation, and/or expression in a cell of a target tissue.

The AAV sequences of the vector typically comprise the cis-acting 5′ and 3′ inverted terminal repeat sequences (See, e.g., B. J. Carter, in “Handbook of Parvoviruses”, ed., P. Tijsser, CRC Press, pp. 155 168 (1990)). The ITR sequences are about 145 bp in length. Preferably, substantially the entire sequences encoding the ITRs are used in the molecule, although some degree of minor modification of these sequences is permissible. The ability to modify these ITR sequences is within the skill of the art. (See, e.g., texts such as Sambrook et al, “Molecular Cloning. A Laboratory Manual”, 2d ed., Cold Spring Harbor Laboratory, New York (1989); and K. Fisher et al., J Virol., 70:520 532 (1996)). An example of such a molecule employed in the present disclosure is a “cis-acting” plasmid containing the transgene, in which the selected transgene sequence and associated regulatory elements are flanked by the 5′ and 3′ AAV ITR sequences. The AAV ITR sequences may be obtained from any known AAV, including presently identified mammalian AAV types.

In some embodiments, the disclosure provides a self-complementary AAV vector. As used herein, the term “self-complementary AAV vector” (scAAV) refers to a vector containing a double-stranded vector genome generated by the absence of a terminal resolution site (TR) from one of the ITRs of the AAV. The absence of a TR prevents the initiation of replication at the vector terminus where the TR is not present. In general, scAAV vectors generate single-stranded, inverted repeat genomes, with a wild-type (wt) AAV TR at each end and a mutated TR (mTR) in the middle.

In some embodiments, the rAAVs of the present disclosure are pseudotyped rAAVs. Pseudotyping is the process of producing viruses or viral vectors in combination with foreign viral envelope proteins. The result is a pseudotyped virus particle. With this method, the foreign viral envelope proteins can be used to alter host tropism or an increased/decreased stability of the virus particles. In some aspects, a pseudotyped rAAV comprises nucleic acids from two or more different AAVs, wherein the nucleic acid from one AAV encodes a capsid protein and the nucleic acid of at least one other AAV encodes other viral proteins and/or the viral genome. In some embodiments, a pseudotyped rAAV refers to an AAV comprising an inverted terminal repeat (ITR) of one AAV serotype and a capsid protein of a different AAV serotype. For example, a pseudotyped AAV vector containing the ITRs of serotype X encapsidated with the proteins of Y will be designated as AAVX/Y (e.g., AAV2/1 has the ITRs of AAV2 and the capsid of AAV1). In some embodiments, pseudotyped rAAVs may be useful for combining the tissue-specific targeting capabilities of a capsid protein from one AAV serotype with the viral DNA from another AAV serotype, thereby allowing targeted delivery of a transgene to a target tissue.

In addition to the major elements identified above for the recombinant AAV vector, the vector also includes conventional control elements necessary which are operably linked to the transgene in a manner which permits its transcription, translation and/or expression in a cell transfected with the plasmid vector or infected with the virus produced by the disclosure. As used herein, “operably linked” sequences include both expression control sequences that are contiguous with the gene of interest and expression control sequences that act in trans or at a distance to control the gene of interest.

Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (e.g., Kozak consensus sequence); sequences that enhance protein stability; and when desired, sequences that enhance secretion of the encoded product. A great number of expression control sequences, including promoters that are native, constitutive, inducible and/or tissue-specific, are known in the art and may be utilized.

As used herein, a nucleic acid sequence (e.g., coding sequence) and regulatory sequences are said to be “operably” linked when they are covalently linked in such a way as to place the expression or transcription of the nucleic acid sequence under the influence or control of the regulatory sequences. If it is desired that the nucleic acid sequences be translated into a functional protein, two DNA sequences are said to be operably linked if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably linked to a nucleic acid sequence if the promoter region were capable of effecting transcription of that DNA sequence such that the resulting transcript might be translated into the desired protein or polypeptide. Similarly two or more coding regions are operably linked when they are linked in such a way that their transcription from a common promoter results in the expression of two or more proteins having been translated in frame. In some embodiments, operably linked coding sequences yield a fusion protein. In some embodiments, operably linked coding sequences yield a functional RNA (e.g., shRNA, miRNA, miRNA inhibitor).

For nucleic acids encoding proteins, a polyadenylation sequence generally is inserted following the transgene sequences and before the 3′ AAV ITR sequence. A rAAV construct useful in the present disclosure may also contain an intron, desirably located between the promoter/enhancer sequence and the transgene. One possible intron sequence is derived from SV-40, and is referred to as the SV-40 T intron sequence. Another vector element that may be used is an internal ribosome entry site (IRES). An IRES sequence is used to produce more than one polypeptide from a single gene transcript. An IRES sequence would be used to produce a protein that contains more than one polypeptide chains. Selection of these and other common vector elements are conventional and many such sequences are available [see, e.g., Sambrook et al, and references cited therein at, for example, pages 3.18 3.26 and 16.17 16.27 and Ausubel et al., Current Protocols in Molecular Biology, John Wiley & Sons, New York, 1989]. In some embodiments, a Foot and Mouth Disease Virus 2A sequence is included in polyprotein; this is a small peptide (approximately 18 amino acids in length) that has been shown to mediate the cleavage of polyproteins (Ryan, M D et al., EMBO, 1994; 4: 928-933; Mattion, N M et al., J Virology, November 1996; p. 8124-8127; Furler, S et al., Gene Therapy, 2001; 8: 864-873; and Halpin, C et al., The Plant Journal, 1999; 4: 453-459). The cleavage activity of the 2A sequence has previously been demonstrated in artificial systems including plasmids and gene therapy vectors (AAV and retroviruses) (Ryan, M D et al., EMBO, 1994; 4: 928-933; Mattion, N M et al., J Virology, November 1996; p. 8124-8127; Furler, S et al., Gene Therapy, 2001; 8: 864-873; and Halpin, C et al., The Plant Journal, 1999; 4: 453-459; de Felipe, P et al., Gene Therapy, 1999; 6: 198-208; de Felipe, P et al., Human Gene Therapy, 2000; 11: 1921-1931.; and Klump, H et al., Gene Therapy, 2001; 8: 811-817).

The precise nature of the regulatory sequences needed for gene expression in host cells may vary between species, tissues or cell types, but shall in general include, as necessary, 5′ non-transcribed and 5′ non-translated sequences involved with the initiation of transcription and translation respectively, such as a TATA box, capping sequence, CAAT sequence, enhancer elements, and the like. Especially, such 5′ non-transcribed regulatory sequences will include a promoter region that includes a promoter sequence for transcriptional control of the operably joined gene. Regulatory sequences may also include enhancer sequences or upstream activator sequences as desired. The vectors of the disclosure may optionally include 5′ leader or signal sequences. The choice and design of an appropriate vector is within the ability and discretion of one of ordinary skill in the art.

Examples of constitutive promoters include, without limitation, the retroviral Rous sarcoma virus (RSV) LTR promoter (optionally with the RSV enhancer), the cytomegalovirus (CMV) promoter (optionally with the CMV enhancer) [see, e.g., Boshart et al, Cell, 41:521-530 (1985)], the SV40 promoter, the dihydrofolate reductase promoter, the β-actin promoter, the phosphoglycerol kinase (PGK) promoter, and the EF1α promoter [Invitrogen].

Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clontech and Ariad. Many other systems have been described and can be readily selected by one of skill in the art. Examples of inducible promoters regulated by exogenously supplied promoters include the zinc-inducible sheep metallothionine (MT) promoter, the dexamethasone (Dex)-inducible mouse mammary tumor virus (MMTV) promoter, the T7 polymerase promoter system (WO 98/10088); the ecdysone insect promoter (No et al, Proc. Natl. Acad. Sci. USA, 93:3346-3351 (1996)), the tetracycline-repressible system (Gossen et al, Proc. Natl. Acad. Sci. USA, 89:5547-5551 (1992)), the tetracycline-inducible system (Gossen et al, Science, 268:1766-1769 (1995), see also Harvey et al, Curr. Opin. Chem. Biol., 2:512-518 (1998)), the RU486-inducible system (Wang et al, Nat. Biotech., 15:239-243 (1997) and Wang et al, Gene Ther., 4:432-441 (1997)) and the rapamycin-inducible system (Magari et al, J. Clin. Invest., 100:2865-2872 (1997)). Still other types of inducible promoters that may be useful in this context are those that are regulated by a specific physiological state, e.g., temperature, acute phase, a particular differentiation state of the cell, or in replicating cells only.

In another embodiment, the native promoter for the transgene will be used. The native promoter may be preferred when it is desired that expression of the transgene should mimic the native expression. The native promoter may be used when expression of the transgene must be regulated temporally or developmentally, or in a tissue-specific manner, or in response to specific transcriptional stimuli. In a further embodiment, other native expression control elements, such as enhancer elements, polyadenylation sites or Kozak consensus sequences may also be used to mimic the native expression.

In some embodiments, the regulatory sequences impart tissue-specific gene expression capabilities. In some cases, the tissue-specific regulatory sequences bind tissue-specific transcription factors that induce transcription in a tissue specific manner. Such tissue-specific regulatory sequences (e.g., promoters, enhancers, etc.) are well known in the art. Exemplary tissue-specific regulatory sequences include, but are not limited to the following tissue specific promoters: a liver-specific thyroxin binding globulin (TBG) promoter, an insulin promoter, a glucagon promoter, a somatostatin promoter, a pancreatic polypeptide (PPY) promoter, a synapsin-1 (Syn) promoter, a creatine kinase (MCK) promoter, a mammalian desmin (DES) promoter, a α-myosin heavy chain (a-MHC) promoter, a gastrointestinal-specific mucin-2 promoter, an eye-specific retinoschisin promoter, an eye-specific K12 promoter, a respiratory tissue-specific CC10 promoter, a respiratory tissue-specific surfactant protein C (SP-C) promoter, a breast tissue-specific PRC1 promoter, a breast tissue-specific RRM2 promoter, a urinary tract tissue-specific uroplakin 2 (UPII) promoter, a uterine tissue-specific lactoferrin promoter, or a cardiac Troponin T (cTnT) promoter. Other exemplary promoters include Beta-actin promoter, hepatitis B virus core promoter, Sandig et al., Gene Ther., 3:1002-9 (1996); alpha-fetoprotein (AFP) promoter, Arbuthnot et al., Hum. Gene Ther., 7:1503-14 (1996)), bone osteocalcin promoter (Stein et al., Mol. Biol. Rep., 24:185-96 (1997)); bone sialoprotein promoter (Chen et al., J. Bone Miner. Res., 11:654-64 (1996)), CD2 promoter (Hansal et al., J. Immunol., 161:1063-8 (1998); immunoglobulin heavy chain promoter; T cell receptor α-chain promoter, neuronal such as neuron-specific enolase (NSE) promoter (Andersen et al., Cell. Mol. Neurobiol., 13:503-15 (1993)), neurofilament light-chain gene promoter (Piccioli et al., Proc. Natl. Acad. Sci. USA, 88:5611-5 (1991)), and the neuron-specific vgf gene promoter (Piccioli et al., Neuron, 15:373-84 (1995)), among others which will be apparent to the skilled artisan.

In some embodiments, one or more bindings sites for one or more of miRNAs are incorporated in a transgene of a rAAV vector, to inhibit the expression of the transgene in one or more tissues of an subject harboring the transgene. The skilled artisan will appreciate that binding sites may be selected to control the expression of a transgene in a tissue specific manner. For example, binding sites for the liver-specific miR-122 may be incorporated into a transgene to inhibit expression of that transgene in the liver. The target sites in the mRNA may be in the 5′ UTR, the 3′ UTR or in the coding region. Typically, the target site is in the 3′ UTR of the mRNA. Furthermore, the transgene may be designed such that multiple miRNAs regulate the mRNA by recognizing the same or multiple sites. The presence of multiple miRNA binding sites may result in the cooperative action of multiple RISCs and provide highly efficient inhibition of expression. The target site sequence may comprise a total of 5-100, 10-60, or more nucleotides. The target site sequence may comprise at least 5 nucleotides of the sequence of a target gene binding site.

### Recombinant AAV Vector: Transgene Coding Sequences

The composition of the transgene sequence of the rAAV vector will depend upon the use to which the resulting vector will be put. For example, one type of transgene sequence includes a reporter sequence, which upon expression produces a detectable signal. In another example, the transgene encodes a therapeutic protein or therapeutic functional RNA. In another example, the transgene encodes a protein or functional RNA that is intended to be used for research purposes, e.g., to create a somatic transgenic animal model harboring the transgene, e.g., to study the function of the transgene product. In another example, the transgene encodes a protein or functional RNA that is intended to be used to create an animal model of disease. Appropriate transgene coding sequences will be apparent to the skilled artisan.

Reporter sequences that may be provided in a transgene include, without limitation, DNA sequences encoding β-lactamase, β-galactosidase (LacZ), alkaline phosphatase, thymidine kinase, green fluorescent protein (GFP), chloramphenicol acetyltransferase (CAT), luciferase, and others well known in the art. When associated with regulatory elements which drive their expression, the reporter sequences, provide signals detectable by conventional means, including enzymatic, radiographic, colorimetric, fluorescence or other spectrographic assays, fluorescent activating cell sorting assays and immunological assays, including enzyme linked immunosorbent assay (ELISA), radioimmunoassay (RIA) and immunohistochemistry. For example, where the marker sequence is the LacZ gene, the presence of the vector carrying the signal is detected by assays for β-galactosidase activity. Where the transgene is green fluorescent protein or luciferase, the vector carrying the signal may be measured visually by color or light production in a luminometer. Such reporters can, for example, be useful in verifying the tissue-specific targeting capabilities and tissue specific promoter regulatory activity of an rAAV.

In some aspects, the disclosure provides rAAV vectors for use in methods of preventing or treating one or more genetic deficiencies or dysfunctions in a mammal, such as for example, a polypeptide deficiency or polypeptide excess in a mammal, and particularly for treating or reducing the severity or extent of deficiency in a human manifesting one or more of the disorders linked to a deficiency in such polypeptides in cells and tissues. The method involves administration of an rAAV vector that encodes one or more therapeutic peptides, polypeptides, siRNAs, microRNAs, antisense nucleotides, etc. in a pharmaceutically-acceptable carrier to the subject in an amount and for a period of time sufficient to treat the deficiency or disorder in the subject suffering from such a disorder.

Thus, the disclosure embraces the delivery of rAAV vectors encoding one or more peptides, polypeptides, or proteins, which are useful for the treatment or prevention of disease states in a mammalian subject. Exemplary therapeutic proteins include one or more polypeptides selected from the group consisting of growth factors, interleukins, interferons, anti-apoptosis factors, cytokines, anti-diabetic factors, anti-apoptosis agents, coagulation factors, anti-tumor factors. Other non-limiting examples of therapeutic proteins include BDNF, CNTF, CSF, EGF, FGF, G-SCF, GM-CSF, gonadotropin, IFN, IFG-1, M-CSF, NGF, PDGF, PEDF, TGF, VEGF, TGF-B2, TNF, prolactin, somatotropin, XIAP1, IL-1, IL-2, IL-3, IL-4, IL-5, IL-6, IL-7, IL-8, IL-9, IL-10, IL-10 (187A), viral IL-10, IL-11, IL-12, IL-13, IL-14, IL-15, IL-16 IL-17, and IL-18.

The rAAV vectors may comprise a gene to be transferred to a subject to treat a disease associated with reduced expression, lack of expression or dysfunction of the gene. Exemplary genes and associated disease states include, but are not limited to: glucose-6-phosphatase, associated with glycogen storage deficiency type 1A; phosphoenolpyruvate-carboxykinase, associated with Pepck deficiency; galactose-1 phosphate uridyl transferase, associated with galactosemia; phenylalanine hydroxylase, associated with phenylketonuria; branched chain alpha-ketoacid dehydrogenase, associated with Maple syrup urine disease; fumarylacetoacetate hydrolase, associated with tyrosinemia type 1; methylmalonyl-CoA mutase, associated with methylmalonic acidemia; medium chain acyl CoA dehydrogenase, associated with medium chain acetyl CoA deficiency; omithine transcarbamylase, associated with omithine transcarbamylase deficiency; argininosuccinic acid synthetase, associated with citrullinemia; low density lipoprotein receptor protein, associated with familial hypercholesterolemia; UDP-glucouronosyltransferase, associated with Crigler-Najjar disease; adenosine deaminase, associated with severe combined immunodeficiency disease; hypoxanthine guanine phosphoribosyl transferase, associated with Gout and Lesch-Nyan syndrome; biotinidase, associated with biotinidase deficiency; beta-glucocerebrosidase, associated with Gaucher disease; beta-glucuronidase, associated with Sly syndrome; peroxisome membrane protein 70 kDa, associated with Zellweger syndrome; porphobilinogen deaminase, associated with acute intermittent porphyria; alpha-1 antitrypsin for treatment of alpha-1 antitrypsin deficiency (emphysema); erythropoietin for treatment of anemia due to thalassemia or to renal failure; vascular endothelial growth factor, angiopoietin-1, and fibroblast growth factor for the treatment of ischemic diseases; thrombomodulin and tissue factor pathway inhibitor for the treatment of occluded blood vessels as seen in, for example, atherosclerosis, thrombosis, or embolisms; aromatic amino acid decarboxylase (AADC), and tyrosine hydroxylase (TH) for the treatment of Parkinson's disease; the beta adrenergic receptor, anti-sense to, or a mutant form of, phospholamban, the sarco(endo)plasmic reticulum adenosine triphosphatase-2 (SERCA2), and the cardiac adenylyl cyclase for the treatment of congestive heart failure; a tumor suppessor gene such as p53 for the treatment of various cancers; a cytokine such as one of the various interleukins for the treatment of inflammatory and immune disorders and cancers; dystrophin or minidystrophin and utrophin or miniutrophin for the treatment of muscular dystrophies; and, insulin for the treatment of diabetes.

In some embodiments, the disclosure relates to an AAV comprising a nucleic acid encoding a protein or functional RNA useful for the treatment of a condition, disease or disorder associated with the central nervous system (CNS). The following is a non-limiting list of genes associated with CNS disease: DRD2, GRIA1, GRIA2, GRIN1, SLC1A1, SYP, SYT1, CHRNA7, 3Rtau/4rTUS, APP, BAX, BCL-2, GRIK1, GFAP, IL-1, AGER, associated with Alzheimer's Disease; UCH-L1, SKP1, EGLN1, Nurr-1, BDNF, TrkB, gstm1, S106β, associated with Parkinson's Disease; IT15, PRNP, JPH3, TBP, ATXN1, ATXN2, ATXN3, Atrophin 1, FTL, TITF-1, associated with Huntington's Disease; FXN, associated with Freidrich's ataxia; ASPA, associated with Canavan's Disease; DMD, associated with muscular dystrophy; and SMN1, UBE1, DYNC1H1 associated with spinal muscular atrophy. In some embodiments, the disclosure relates to recombinant AAVs comprising nucleic acids that express one or more of the foregoing genes or fragments thereof. In some embodiments, the disclosure relates to recombinant AAVs comprising nucleic acids that express one or more functional RNAs that inhibit expression of one or more of the foregoing genes.

In some embodiments, the disclosure relates to a nucleic acid encoding a protein or functional RNA useful for the treatment of a condition, disease or disorder associated with the cardiovascular system. The following is a non-limiting list of genes associated with cardiovascular disease: VEGF, FGF, SDF-1, connexin 40, connexin 43, SCN4a, HIF1α, SERCa2a, ADCY1, and ADCY6. In some embodiments, the disclosure relates to recombinant AAVs comprising nucleic acids that express one or more of the foregoing genes or fragments thereof. In some embodiments, the disclosure relates to recombinant AAVs comprising nucleic acids that express one or more functional RNAs that inhibit expression of one or more of the foregoing genes.

In some embodiments, the disclosure relates to an AAV comprising a nucleic acid encoding a protein or functional RNA useful for the treatment of a condition, disease or disorder associated with the pulmonary system. The following is a non-limiting list of genes associated with pulmonary disease: TNFα, TGFβ1, SFTPA1, SFTPA2, SFTPB, SFTPC, HPS1, HPS3, HPS4, ADTB3A, IL1A, IL1B, LTA, IL6, CXCR1, and CXCR2. In some embodiments, the disclosure relates to recombinant AAVs comprising nucleic acids that express one or more of the foregoing genes or fragments thereof. In some embodiments, the disclosure relates to recombinant AAVs comprising nucleic acids that express one or more functional RNAs that inhibit expression of one or more of the foregoing genes.

In some embodiments, the disclosure relates to an AAV comprising a nucleic acid encoding a protein or functional RNA useful for the treatment of a condition, disease or disorder associated with the liver. The following is a non-limiting list of genes associated with liver disease: α1-AT, HFE, ATP7B, fumarylacetoacetate hydrolase (FAH), glucose-6-phosphatase, NCAN, GCKR, LYPLAL1, and PNPLA3. In some embodiments, the disclosure relates to recombinant AAVs comprising nucleic acids that express one or more of the foregoing genes or fragments thereof. In some embodiments, the disclosure relates to recombinant AAVs comprising nucleic acids that express one or more functional RNAs that inhibit expression of one or more of the foregoing genes.

In some embodiments, the disclosure relates to an AAV comprising a nucleic acid encoding a protein or functional RNA useful for the treatment of a condition, disease or disorder associated with the kidney. The following is a non-limiting list of genes associated with kidney disease: PKD1, PKD2, PKHD1, NPHS1, NPHS2, PLCE1, CD2AP, LAMB2, TRPC6, WT1, LMX1B, SMARCAL1, COQ2, PDSS2, SCARB3, FN1, COL4A5, COL4A6, COL4A3, COL4A4, FOX1C, RET, UPK3A, BMP4, SIX2, CDC5L, USF2, ROBO2, SLIT2, EYA1, MYOG, SIX1, SIXS, FRAS1, FREM2, GATA3, KAL1, PAX2, TCF2, and SALL1. In some embodiments, the disclosure relates to recombinant AAVs comprising nucleic acids that express one or more of the foregoing genes or fragments thereof. In some embodiments, the disclosure relates to recombinant AAVs comprising nucleic acids that express one or more functional RNAs that inhibit expression of one or more of the foregoing genes.

In some embodiments, the disclosure relates to an AAV comprising a nucleic acid encoding a protein or functional RNA useful for the treatment of a condition, disease or disorder associated with the eye. The following is a non-limiting list of genes associated with ocular disease: CFH, C3, MT-ND2, ARMS2, TIMP3, CAMK4, FMN1, RHO, USH2A, RPGR, RP2, TMCO, SIX1, SIX6, LRP12, ZFPM2, TBK1, GALC, myocilin, CYP1B1, CAV1, CAV2, optineurin and CDKN2B. In some embodiments, the disclosure relates to recombinant AAVs comprising nucleic acids that express one or more of the foregoing genes or fragments thereof. In some embodiments, the disclosure relates to recombinant AAVs comprising nucleic acids that express one or more functional RNAs that inhibit expression of one or more of the foregoing genes.

In some embodiments, the disclosure relates to an AAV comprising a nucleic acid encoding a protein or functional RNA useful for the treatment of a condition, disease or disorder associated with breast. The following is a non-limiting list of genes associated with breast disease: BRCA1, BRCA2, Tp53, PTEN, HER2, BRAF, and PARP1. In some embodiments, the disclosure relates to recombinant AAVs comprising nucleic acids that express one or more of the foregoing genes or fragments thereof. In some embodiments, the disclosure relates to recombinant AAVs comprising nucleic acids that express one or more functional RNAs that inhibit expression of one or more of the foregoing genes.

In some embodiments, the disclosure relates to an AAV comprising a nucleic acid encoding a protein or functional RNA useful for the treatment of a condition, disease or disorder associated with the gastrointestinal tract. The following is a non-limiting list of genes associated with gastrointestinal disease: CYP2C19, CCL26, APC, IL12, IL10, and IL-18. In some embodiments, the disclosure relates to recombinant AAVs comprising nucleic acids that express one or more of the foregoing genes or fragments thereof. In some embodiments, the disclosure relates to recombinant AAVs comprising nucleic acids that express one or more functional RNAs that inhibit expression of one or more of the foregoing genes.

In some embodiments, the disclosure relates to an AAV comprising a nucleic acid encoding a protein or functional RNA useful for the treatment of a condition, disease or disorder associated with the pancreas. The following is a non-limiting list of genes associated with pancreatic disease: PRSS1, SPINK1, STK11, MLH1, KRAS2, p16, p53, and BRAF. In some embodiments, the disclosure relates to recombinant AAVs comprising nucleic acids that express one or more of the foregoing genes or fragments thereof. In some embodiments, the disclosure relates to recombinant AAVs comprising nucleic acids that express one or more functional RNAs that inhibit expression of one or more of the foregoing genes.

In some embodiments, the disclosure relates to an AAV comprising a nucleic acid encoding a protein or functional RNA useful for the treatment of a condition, disease or disorder associated with the urinary tract. The following is a non-limiting list of genes associated with urinary tract disease: HSPA1B, CXCR1 & 2, TLR2, TLR4, TGF-1, FGFR3, RB1, HRAS, TP53, and TSC1. In some embodiments, the disclosure relates to recombinant AAVs comprising nucleic acids that express one or more of the foregoing genes or fragments thereof. In some embodiments, the disclosure relates to recombinant AAVs comprising nucleic acids that express one or more functional RNAs that inhibit expression of one or more of the foregoing genes.

In some embodiments, the disclosure relates to an AAV comprising a nucleic acid encoding a protein or functional RNA useful for the treatment of a condition, disease or disorder associated with the uterus. The following is a non-limiting list of genes associated with ocular disease: DN-ER, MLH1, MSH2, MSH6, PMS1, and PMS2. In some embodiments, the disclosure relates to recombinant AAVs comprising nucleic acids that express one or more of the foregoing genes or fragments thereof. In some embodiments, the disclosure relates to recombinant AAVs comprising nucleic acids that express one or more functional RNAs that inhibit expression of one or more of the foregoing genes.

The rAAVs of the disclosure can be used to restore the expression of genes that are reduced in expression, silenced, or otherwise dysfunctional in a subject (e.g., a tumor suppressor that has been silenced in a subject having cancer). The rAAVs of the disclosure can also be used to knockdown the expression of genes that are aberrantly expressed in a subject (e.g., an oncogene that is expressed in a subject having cancer). In some embodiments, an rAAV vector comprising a nucleic acid encoding a gene product associated with cancer (e.g., tumor suppressors) may be used to treat the cancer, by administering a rAAV harboring the rAAV vector to a subject having the cancer. In some embodiments, an rAAV vector comprising a nucleic acid encoding a small interfering nucleic acid (e.g., shRNAs, miRNAs) that inhibits the expression of a gene product associated with cancer (e.g., oncogenes) may be used to treat the cancer, by administering a rAAV harboring the rAAV vector to a subject having the cancer. In some embodiments, an rAAV vector comprising a nucleic acid encoding a gene product associated with cancer (or a functional RNA that inhibits the expression of a gene associated with cancer) may be used for research purposes, e.g., to study the cancer or to identify therapeutics that treat the cancer. The following is a non-limiting list of exemplary genes known to be associated with the development of cancer (e.g., oncogenes and tumor suppressors): AARS, ABCB1, ABCC4, ABI2, ABL1, ABL2, ACK1, ACP2, ACY1, ADSL, AK1, AKR1C2, AKT1, ALB, ANPEP, ANXA5, ANXA7, AP2M1, APC, ARHGAP5, ARHGEF5, ARID4A, ASNS, ATF4, ATM, ATP5B, ATP5O, AXL, BARD1, BAX, BCL2, BHLHB2, BLMH, BRAF, BRCA1, BRCA2, BTK, CANX, CAP1, CAPN1, CAPNS1, CAV1, CBFB, CBLB, CCL2, CCND1, CCND2, CCND3, CCNE1, CCT5, CCYR61, CD24, CD44, CD59, CDC20, CDC25, CDC25A, CDC25B, CDC2L5, CDK10, CDK4, CDK5, CDK9, CDKL1, CDKN1A, CDKN1B, CDKN1C, CDKN2A, CDKN2B, CDKN2D, CEBPG, CENPC1, CGRRF1, CHAF1A, CIB1, CKMT1, CLK1, CLK2, CLK3, CLNS1A, CLTC, COL1A1, COL6A3, COX6C, COX7A2, CRAT, CRHR1, CSF1R, CSK, CSNK1G2, CTNNA1, CTNNB1, CTPS, CTSC, CTSD, CUL1, CYR61, DCC, DCN, DDX10, DEK, DHCR7, DHRS2, DHX8, DLG3, DVL1, DVL3, E2F1, E2F3, E2F5, EGFR, EGR1, EIF5, EPHA2, ERBB2, ERBB3, ERBB4, ERCC3, ETV1, ETV3, ETV6, F2R, FASTK, FBN1, FBN2, FES, FGFR1, FGR, FKBP8, FN1, FOS, FOSL1, FOSL2, FOXG1A, FOXO1A, FRAP1, FRZB, FTL, FZD2, FZD5, FZD9, G22P1, GAS6, GCN5L2, GDF15, GNA13, GNAS, GNB2, GNB2L1, GPR39, GRB2, GSK3A, GSPT1, GTF2I, HDAC1, HDGF, HMMR, HPRT1, HRB, HSPA4, HSPA5, HSPA8, HSPB1, HSPH1, HYAL1, HYOU1, ICAM1, ID1, ID2, IDUA, IER3, IFITM1, IGF1R, IGF2R, IGFBP3, IGFBP4, IGFBP5, IL1B, ILK, ING1, IRF3, ITGA3, ITGA6, ITGB4, JAK1, JARID1A, JUN, JUNB, JUND, K-ALPHA-1, KIT, KITLG, KLK10, KPNA2, KRAS2, KRT18, KRT2A, KRT9, LAMB1, LAMP2, LCK, LCN2, LEP, LITAF, LRPAP1, LTF, LYN, LZTR1, MADH1, MAP2K2, MAP3K8, MAPK12, MAPK13, MAPKAPK3, MAPRE1, MARS, MAS1, MCC, MCM2, MCM4, MDM2, MDM4, MET, MGST1, MICB, MLLT3, MME, MMP1, MMP14, MMP17, MMP2, MNDA, MSH2, MSH6, MT3, MYB, MYBL1, MYBL2, MYC, MYCL1, MYCN, MYD88, MYL9, MYLK, NEO1, NF1, NF2, NFKB1, NFKB2, NFSF7, NID, NINJ1, NMBR, NME1, NME2, NME3, NOTCH1, NOTCH2, NOTCH4, NPM1, NQO1, NR1D1, NR2F1, NR2F6, NRAS, NRG1, NSEP1, OSM, PA2G4, PABPC1, PCNA, PCTK1, PCTK2, PCTK3, PDGFA, PDGFB, PDGFRA, PDPK1, PEA15, PFDN4, PFDN5, PGAM1, PHB, PIK3CA, PIK3CB, PIK3CG, PIM1, PKM2, PKMYT1, PLK2, PPARD, PPARG, PPIH, PPP1CA, PPP2R5A, PRDX2, PRDX4, PRKAR1A, PRKCBP1, PRNP, PRSS15, PSMA1, PTCH, PTEN, PTGS1, PTMA, PTN, PTPRN, RAB5A, RAC1, RAD50, RAF1, RALBP1, RAP1A, RARA, RARB, RASGRF1, RB1, RBBP4, RBL2, REA, REL, RELA, RELB, RET, RFC2, RGS19, RHOA, RHOB, RHOC, RHOD, RIPK1, RPN2, RPS6KB1, RRM1, SARS, SELENBP1, SEMA3C, SEMA4D, SEPP1, SERPINH1, SFN, SFPQ, SFRS7, SHB, SHH, SIAH2, SIVA, SIVA TP53, SKI, SKIL, SLC16A1, SLC1A4, SLC20A1, SMO, SMPD1, SNAI2, SND1, SNRPB2, SOCS1, SOCS3, SOD1, SORT1, SPINT2, SPRY2, SRC, SRPX, STAT1, STAT2, STAT3, STAT5B, STC1, TAF1, TBL3, TBRG4, TCF1, TCF7L2, TFAP2C, TFDP1, TFDP2, TGFA, TGFB1, TGFBI, TGFBR2, TGFBR3, THBS1, TIE, TIMP1, TIMP3, TJP1, TK1, TLE1, TNF, TNFRSF10A, TNFRSF10B, TNFRSF1A, TNFRSF1B, TNFRSF6, TNFSF7, TNK1, TOB1, TP53, TP53BP2, TP53I3, TP73, TPBG, TPT1, TRADD, TRAM1, TRRAP, TSG101, TUFM, TXNRD1, TYRO3, UBC, UBE2L6, UCHL1, USP7, VDAC1, VEGF, VHL, VIL2, WEE1, WNT1, WNT2, WNT2B, WNT3, WNT5A, WT1, XRCC1, YES1, YWHAB, YWHAZ, ZAP70, and ZNF9.

A rAAV vector may comprise as a transgene, a nucleic acid encoding a protein or functional RNA that modulates apoptosis. The following is a non-limiting list of genes associated with apoptosis and nucleic acids encoding the products of these genes and their homologues and encoding small interfering nucleic acids (e.g., shRNAs, miRNAs) that inhibit the expression of these genes and their homologues are useful as transgenes in certain embodiments of the disclosure: RPS27A, ABL1, AKT1, APAF1, BAD, BAG1, BAG3, BAG4, BAK1, BAX, BCL10, BCL2, BCL2A1, BCL2L1, BCL2L10, BCL2L11, BCL2L12, BCL2L13, BCL2L2, BCLAF1, BFAR, BID, BIK, NAIP, BIRC2, BIRC3, XIAP, BIRC5, BIRC6, BIRC7, BIRC8, BNIP1, BNIP2, BNIP3, BNIP3L, BOK, BRAF, CARD10, CARD11, NLRC4, CARD14, NOD2, NOD1, CARD6, CARD8, CARD9, CASP1, CASP10, CASP14, CASP2, CASP3, CASP4, CASP5, CASP6, CASP7, CASP8, CASP9, CFLAR, CIDEA, CIDEB, CRADD, DAPK1, DAPK2, DFFA, DFFB, FADD, GADD45A, GDNF, HRK, IGF1R, LTA, LTBR, MCL1, NOL3, PYCARD, RIPK1, RIPK2, TNF, TNFRSF10A, TNFRSF10B, TNFRSF10C, TNFRSF10D, TNFRSF11B, TNFRSF12A, TNFRSF14, TNFRSF19, TNFRSF1A, TNFRSF1B, TNFRSF21, TNFRSF25, CD40, FAS, TNFRSF6B, CD27, TNFRSF9, TNFSF10, TNFSF14, TNFSF18, CD40LG, FASLG, CD70, TNFSF8, TNFSF9, TP53, TP53BP2, TP73, TP63, TRADD, TRAF1, TRAF2, TRAF3, TRAF4, TRAF5 DRD2, GRIA1, GRIA2, GRIN1, SLC1A1, SYP, SYT1, CHRNA7, 3Rtau/4rTUS, APP, BAX, BCL-2, GRIK1, GFAP, IL-1, AGER, UCH-L1, SKP1, EGLN1, Nurr-1, BDNF, TrkB, gstm1, S106β, IT15, PRNP, JPH3, TBP, ATXN1, ATXN2, ATXN3, Atrophin 1, FTL, TITF-1, FXN, ASPA, DMD, and SMN1, UBE1, DYNC1H1.

The skilled artisan will also realize that in the case of transgenes encoding proteins or polypeptides, that mutations that results in conservative amino acid substitutions may be made in a transgene to provide functionally equivalent variants, or homologs of a protein or polypeptide. In some aspects the disclosure embraces sequence alterations that result in conservative amino acid substitution of a transgene. In some embodiments, the transgene comprises a gene having a dominant negative mutation. For example, a transgene may express a mutant protein that interacts with the same elements as a wild-type protein, and thereby blocks some aspect of the function of the wild-type protein.

Useful transgene products also include miRNAs. miRNAs and other small interfering nucleic acids regulate gene expression via target RNA transcript cleavage/degradation or translational repression of the target messenger RNA (mRNA). miRNAs are natively expressed, typically as final 19-25 non-translated RNA products. miRNAs exhibit their activity through sequence-specific interactions with the 3′ untranslated regions (UTR) of target mRNAs. These endogenously expressed miRNAs form hairpin precursors that are subsequently processed into a miRNA duplex, and further into a “mature” single stranded miRNA molecule. This mature miRNA guides a multiprotein complex, miRISC, which identifies target site, e.g., in the 3′ UTR regions, of target mRNAs based upon their complementarity to the mature miRNA.

The following non-limiting list of miRNA genes, and their homologues, are useful as transgenes or as targets for small interfering nucleic acids encoded by transgenes (e.g., miRNA sponges, antisense oligonucleotides, TuD RNAs) in certain embodiments of the methods: hsa-let-7a, hsa-let-7a*, hsa-let-7b, hsa-let-7b*, hsa-let-7c, hsa-let-7c*, hsa-let-7d, hsa-let-7d*, hsa-let-7e, hsa-let-7e*, hsa-let-7f, hsa-let-7f-1*, hsa-let-7f-2*, hsa-let-7g, hsa-let-7g*, hsa-let-7i, hsa-let-7i*, hsa-miR-1, hsa-miR-100, hsa-miR-100*, hsa-miR-101, hsa-miR-101*, hsa-miR-103, hsa-miR-105, hsa-miR-105*, hsa-miR-106a, hsa-miR-106a*, hsa-miR-106b, hsa-miR-106b*, hsa-miR-107, hsa-miR-10a, hsa-miR-10a*, hsa-miR-10b, hsa-miR-10b*, hsa-miR-1178, hsa-miR-1179, hsa-miR-1180, hsa-miR-1181, hsa-miR-1182, hsa-miR-1183, hsa-miR-1184, hsa-miR-1185, hsa-miR-1197, hsa-miR-1200, hsa-miR-1201, hsa-miR-1202, hsa-miR-1203, hsa-miR-1204, hsa-miR-1205, hsa-miR-1206, hsa-miR-1207-3p, hsa-miR-1207-5p, hsa-miR-1208, hsa-miR-122, hsa-miR-122*, hsa-miR-1224-3p, hsa-miR-1224-5p, hsa-miR-1225-3p, hsa-miR-1225-5p, hsa-miR-1226, hsa-miR-1226*, hsa-miR-1227, hsa-miR-1228, hsa-miR-1228*, hsa-miR-1229, hsa-miR-1231, hsa-miR-1233, hsa-miR-1234, hsa-miR-1236, hsa-miR-1237, hsa-miR-1238, hsa-miR-124, hsa-miR-124*, hsa-miR-1243, hsa-miR-1244, hsa-miR-1245, hsa-miR-1246, hsa-miR-1247, hsa-miR-1248, hsa-miR-1249, hsa-miR-1250, hsa-miR-1251, hsa-miR-1252, hsa-miR-1253, hsa-miR-1254, hsa-miR-1255a, hsa-miR-1255b, hsa-miR-1256, hsa-miR-1257, hsa-miR-1258, hsa-miR-1259, hsa-miR-125a-3p, hsa-miR-125a-5p, hsa-miR-125b, hsa-miR-125b-1*, hsa-miR-125b-2*, hsa-miR-126, hsa-miR-126*, hsa-miR-1260, hsa-miR-1261, hsa-miR-1262, hsa-miR-1263, hsa-miR-1264, hsa-miR-1265, hsa-miR-1266, hsa-miR-1267, hsa-miR-1268, hsa-miR-1269, hsa-miR-1270, hsa-miR-1271, hsa-miR-1272, hsa-miR-1273, hsa-miR-127-3p, hsa-miR-1274a, hsa-miR-1274b, hsa-miR-1275, hsa-miR-127-5p, hsa-miR-1276, hsa-miR-1277, hsa-miR-1278, hsa-miR-1279, hsa-miR-128, hsa-miR-1280, hsa-miR-1281, hsa-miR-1282, hsa-miR-1283, hsa-miR-1284, hsa-miR-1285, hsa-miR-1286, hsa-miR-1287, hsa-miR-1288, hsa-miR-1289, hsa-miR-129*, hsa-miR-1290, hsa-miR-1291, hsa-miR-1292, hsa-miR-1293, hsa-miR-129-3p, hsa-miR-1294, hsa-miR-1295, hsa-miR-129-5p, hsa-miR-1296, hsa-miR-1297, hsa-miR-1298, hsa-miR-1299, hsa-miR-1300, hsa-miR-1301, hsa-miR-1302, hsa-miR-1303, hsa-miR-1304, hsa-miR-1305, hsa-miR-1306, hsa-miR-1307, hsa-miR-1308, hsa-miR-130a, hsa-miR-130a*, hsa-miR-130b, hsa-miR-130b*, hsa-miR-132, hsa-miR-132*, hsa-miR-1321, hsa-miR-1322, hsa-miR-1323, hsa-miR-1324, hsa-miR-133a, hsa-miR-133b, hsa-miR-134, hsa-miR-135a, hsa-miR-135a*, hsa-miR-135b, hsa-miR-135b*, hsa-miR-136, hsa-miR-136*, hsa-miR-137, hsa-miR-138, hsa-miR-138-1*, hsa-miR-138-2*, hsa-miR-139-3p, hsa-miR-139-5p, hsa-miR-140-3p, hsa-miR-140-5p, hsa-miR-141, hsa-miR-141*, hsa-miR-142-3p, hsa-miR-142-5p, hsa-miR-143, hsa-miR-143*, hsa-miR-144, hsa-miR-144*, hsa-miR-145, hsa-miR-145*, hsa-miR-146a, hsa-miR-146a*, hsa-miR-146b-3p, hsa-miR-146b-5p, hsa-miR-147, hsa-miR-147b, hsa-miR-148a, hsa-miR-148a*, hsa-miR-148b, hsa-miR-148b*, hsa-miR-149, hsa-miR-149*, hsa-miR-150, hsa-miR-150*, hsa-miR-151-3p, hsa-miR-151-5p, hsa-miR-152, hsa-miR-153, hsa-miR-154, hsa-miR-154*, hsa-miR-155, hsa-miR-155*, hsa-miR-15a, hsa-miR-15a*, hsa-miR-15b, hsa-miR-15b*, hsa-miR-16, hsa-miR-16-1*, hsa-miR-16-2*, hsa-miR-17, hsa-miR-17*, hsa-miR-181a, hsa-miR-181a*, hsa-miR-181a-2*, hsa-miR-181b, hsa-miR-181c, hsa-miR-181c*, hsa-miR-181d, hsa-miR-182, hsa-miR-182*, hsa-miR-1825, hsa-miR-1826, hsa-miR-1827, hsa-miR-183, hsa-miR-183*, hsa-miR-184, hsa-miR-185, hsa-miR-185*, hsa-miR-186, hsa-miR-186*, hsa-miR-187, hsa-miR-187*, hsa-miR-188-3p, hsa-miR-188-5p, hsa-miR-18a, hsa-miR-18a*, hsa-miR-18b, hsa-miR-18b*, hsa-miR-190, hsa-miR-190b, hsa-miR-191, hsa-miR-191*, hsa-miR-192, hsa-miR-192*, hsa-miR-193a-3p, hsa-miR-193a-5p, hsa-miR-193b, hsa-miR-193b*, hsa-miR-194, hsa-miR-194*, hsa-miR-195, hsa-miR-195*, hsa-miR-196a, hsa-miR-196a*, hsa-miR-196b, hsa-miR-197, hsa-miR-198, hsa-miR-199a-3p, hsa-miR-199a-5p, hsa-miR-199b-5p, hsa-miR-19a, hsa-miR-19a*, hsa-miR-19b, hsa-miR-19b-1*, hsa-miR-19b-2*, hsa-miR-200a, hsa-miR-200a*, hsa-miR-200b, hsa-miR-200b*, hsa-miR-200c, hsa-miR-200c*, hsa-miR-202, hsa-miR-202*, hsa-miR-203, hsa-miR-204, hsa-miR-205, hsa-miR-206, hsa-miR-208a, hsa-miR-208b, hsa-miR-20a, hsa-miR-20a*, hsa-miR-20b, hsa-miR-20b*, hsa-miR-21, hsa-miR-21*, hsa-miR-210, hsa-miR-211, hsa-miR-212, hsa-miR-214, hsa-miR-214*, hsa-miR-215, hsa-miR-216a, hsa-miR-216b, hsa-miR-217, hsa-miR-218, hsa-miR-218-1*, hsa-miR-218-2*, hsa-miR-219-1-3p, hsa-miR-219-2-3p, hsa-miR-219-5p, hsa-miR-22, hsa-miR-22*, hsa-miR-220a, hsa-miR-220b, hsa-miR-220c, hsa-miR-221, hsa-miR-221*, hsa-miR-222, hsa-miR-222*, hsa-miR-223, hsa-miR-223*, hsa-miR-224, hsa-miR-23a, hsa-miR-23a*, hsa-miR-23b, hsa-miR-23b*, hsa-miR-24, hsa-miR-24-1*, hsa-miR-24-2*, hsa-miR-25, hsa-miR-25*, hsa-miR-26a, hsa-miR-26a-1*, hsa-miR-26a-2*, hsa-miR-26b, hsa-miR-26b*, hsa-miR-27a, hsa-miR-27a*, hsa-miR-27b, hsa-miR-27b*, hsa-miR-28-3p, hsa-miR-28-5p, hsa-miR-296-3p, hsa-miR-296-5p, hsa-miR-297, hsa-miR-298, hsa-miR-299-3p, hsa-miR-299-5p, hsa-miR-29a, hsa-miR-29a*, hsa-miR-29b, hsa-miR-29b-1*, hsa-miR-29b-2*, hsa-miR-29c, hsa-miR-29c*, hsa-miR-300, hsa-miR-301a, hsa-miR-301b, hsa-miR-302a, hsa-miR-302a*, hsa-miR-302b, hsa-miR-302b*, hsa-miR-302c, hsa-miR-302c*, hsa-miR-302d, hsa-miR-302d*, hsa-miR-302e, hsa-miR-302f, hsa-miR-30a, hsa-miR-30a*, hsa-miR-30b, hsa-miR-30b*, hsa-miR-30c, hsa-miR-30c-1*, hsa-miR-30c-2*, hsa-miR-30d, hsa-miR-30d*, hsa-miR-30e, hsa-miR-30e*, hsa-miR-31, hsa-miR-31*, hsa-miR-32, hsa-miR-32*, hsa-miR-320a, hsa-miR-320b, hsa-miR-320c, hsa-miR-320d, hsa-miR-323-3p, hsa-miR-323-5p, hsa-miR-324-3p, hsa-miR-324-5p, hsa-miR-325, hsa-miR-326, hsa-miR-328, hsa-miR-329, hsa-miR-330-3p, hsa-miR-330-5p, hsa-miR-331-3p, hsa-miR-331-5p, hsa-miR-335, hsa-miR-335*, hsa-miR-337-3p, hsa-miR-337-5p, hsa-miR-338-3p, hsa-miR-338-5p, hsa-miR-339-3p, hsa-miR-339-5p, hsa-miR-33a, hsa-miR-33a*, hsa-miR-33b, hsa-miR-33b*, hsa-miR-340, hsa-miR-340*, hsa-miR-342-3p, hsa-miR-342-5p, hsa-miR-345, hsa-miR-346, hsa-miR-34a, hsa-miR-34a*, hsa-miR-34b, hsa-miR-34b*, hsa-miR-34c-3p, hsa-miR-34c-5p, hsa-miR-361-3p, hsa-miR-361-5p, hsa-miR-362-3p, hsa-miR-362-5p, hsa-miR-363, hsa-miR-363*, hsa-miR-365, hsa-miR-367, hsa-miR-367*, hsa-miR-369-3p, hsa-miR-369-5p, hsa-miR-370, hsa-miR-371-3p, hsa-miR-371-5p, hsa-miR-372, hsa-miR-373, hsa-miR-373*, hsa-miR-374a, hsa-miR-374a*, hsa-miR-374b, hsa-miR-374b*, hsa-miR-375, hsa-miR-376a, hsa-miR-376a*, hsa-miR-376b, hsa-miR-376c, hsa-miR-377, hsa-miR-377*, hsa-miR-378, hsa-miR-378*, hsa-miR-379, hsa-miR-379*, hsa-miR-380, hsa-miR-380*, hsa-miR-381, hsa-miR-382, hsa-miR-383, hsa-miR-384, hsa-miR-409-3p, hsa-miR-409-5p, hsa-miR-410, hsa-miR-411, hsa-miR-411*, hsa-miR-412, hsa-miR-421, hsa-miR-422a, hsa-miR-423-3p, hsa-miR-423-5p, hsa-miR-424, hsa-miR-424*, hsa-miR-425, hsa-miR-425*, hsa-miR-429, hsa-miR-431, hsa-miR-431*, hsa-miR-432, hsa-miR-432*, hsa-miR-433, hsa-miR-448, hsa-miR-449a, hsa-miR-449b, hsa-miR-450a, hsa-miR-450b-3p, hsa-miR-450b-5p, hsa-miR-451, hsa-miR-452, hsa-miR-452*, hsa-miR-453, hsa-miR-454, hsa-miR-454*, hsa-miR-455-3p, hsa-miR-455-5p, hsa-miR-483-3p, hsa-miR-483-5p, hsa-miR-484, hsa-miR-485-3p, hsa-miR-485-5p, hsa-miR-486-3p, hsa-miR-486-5p, hsa-miR-487a, hsa-miR-487b, hsa-miR-488, hsa-miR-488*, hsa-miR-489, hsa-miR-490-3p, hsa-miR-490-5p, hsa-miR-491-3p, hsa-miR-491-5p, hsa-miR-492, hsa-miR-493, hsa-miR-493*, hsa-miR-494, hsa-miR-495, hsa-miR-496, hsa-miR-497, hsa-miR-497*, hsa-miR-498, hsa-miR-499-3p, hsa-miR-499-5p, hsa-miR-500, hsa-miR-500*, hsa-miR-501-3p, hsa-miR-501-5p, hsa-miR-502-3p, hsa-miR-502-5p, hsa-miR-503, hsa-miR-504, hsa-miR-505, hsa-miR-505*, hsa-miR-506, hsa-miR-507, hsa-miR-508-3p, hsa-miR-508-5p, hsa-miR-509-3-5p, hsa-miR-509-3p, hsa-miR-509-5p, hsa-miR-510, hsa-miR-511, hsa-miR-512-3p, hsa-miR-512-5p, hsa-miR-513a-3p, hsa-miR-513a-5p, hsa-miR-513b, hsa-miR-513c, hsa-miR-514, hsa-miR-515-3p, hsa-miR-515-5p, hsa-miR-516a-3p, hsa-miR-516a-5p, hsa-miR-516b, hsa-miR-517*, hsa-miR-517a, hsa-miR-517b, hsa-miR-517c, hsa-miR-518a-3p, hsa-miR-518a-5p, hsa-miR-518b, hsa-miR-518c, hsa-miR-518c*, hsa-miR-518d-3p, hsa-miR-518d-5p, hsa-miR-518e, hsa-miR-518e*, hsa-miR-518f, hsa-miR-518f*, hsa-miR-519a, hsa-miR-519b-3p, hsa-miR-519c-3p, hsa-miR-519d, hsa-miR-519e, hsa-miR-519e*, hsa-miR-520a-3p, hsa-miR-520a-5p, hsa-miR-520b, hsa-miR-520c-3p, hsa-miR-520d-3p, hsa-miR-520d-5p, hsa-miR-520e, hsa-miR-520f, hsa-miR-520g, hsa-miR-520h, hsa-miR-521, hsa-miR-522, hsa-miR-523, hsa-miR-524-3p, hsa-miR-524-5p, hsa-miR-525-3p, hsa-miR-525-5p, hsa-miR-526b, hsa-miR-526b*, hsa-miR-532-3p, hsa-miR-532-5p, hsa-miR-539, hsa-miR-541, hsa-miR-541*, hsa-miR-542-3p, hsa-miR-542-5p, hsa-miR-543, hsa-miR-544, hsa-miR-545, hsa-miR-545*, hsa-miR-548a-3p, hsa-miR-548a-5p, hsa-miR-548b-3p, hsa-miR-548b-5p, hsa-miR-548c-3p, hsa-miR-548c-5p, hsa-miR-548d-3p, hsa-miR-548d-5p, hsa-miR-548e, hsa-miR-548f, hsa-miR-548g, hsa-miR-548h, hsa-miR-548i, hsa-miR-548j, hsa-miR-548k, hsa-miR-548l, hsa-miR-548m, hsa-miR-548n, hsa-miR-548o, hsa-miR-548p, hsa-miR-549, hsa-miR-550, hsa-miR-550*, hsa-miR-551a, hsa-miR-551b, hsa-miR-551b*, hsa-miR-552, hsa-miR-553, hsa-miR-554, hsa-miR-555, hsa-miR-556-3p, hsa-miR-556-5p, hsa-miR-557, hsa-miR-558, hsa-miR-559, hsa-miR-561, hsa-miR-562, hsa-miR-563, hsa-miR-564, hsa-miR-566, hsa-miR-567, hsa-miR-568, hsa-miR-569, hsa-miR-570, hsa-miR-571, hsa-miR-572, hsa-miR-573, hsa-miR-574-3p, hsa-miR-574-5p, hsa-miR-575, hsa-miR-576-3p, hsa-miR-576-5p, hsa-miR-577, hsa-miR-578, hsa-miR-579, hsa-miR-580, hsa-miR-581, hsa-miR-582-3p, hsa-miR-582-5p, hsa-miR-583, hsa-miR-584, hsa-miR-585, hsa-miR-586, hsa-miR-587, hsa-miR-588, hsa-miR-589, hsa-miR-589*, hsa-miR-590-3p, hsa-miR-590-5p, hsa-miR-591, hsa-miR-592, hsa-miR-593, hsa-miR-593*, hsa-miR-595, hsa-miR-596, hsa-miR-597, hsa-miR-598, hsa-miR-599, hsa-miR-600, hsa-miR-601, hsa-miR-602, hsa-miR-603, hsa-miR-604, hsa-miR-605, hsa-miR-606, hsa-miR-607, hsa-miR-608, hsa-miR-609, hsa-miR-610, hsa-miR-611, hsa-miR-612, hsa-miR-613, hsa-miR-614, hsa-miR-615-3p, hsa-miR-615-5p, hsa-miR-616, hsa-miR-616*, hsa-miR-617, hsa-miR-618, hsa-miR-619, hsa-miR-620, hsa-miR-621, hsa-miR-622, hsa-miR-623, hsa-miR-624, hsa-miR-624*, hsa-miR-625, hsa-miR-625*, hsa-miR-626, hsa-miR-627, hsa-miR-628-3p, hsa-miR-628-5p, hsa-miR-629, hsa-miR-629*, hsa-miR-630, hsa-miR-631, hsa-miR-632, hsa-miR-633, hsa-miR-634, hsa-miR-635, hsa-miR-636, hsa-miR-637, hsa-miR-638, hsa-miR-639, hsa-miR-640, hsa-miR-641, hsa-miR-642, hsa-miR-643, hsa-miR-644, hsa-miR-645, hsa-miR-646, hsa-miR-647, hsa-miR-648, hsa-miR-649, hsa-miR-650, hsa-miR-651, hsa-miR-652, hsa-miR-653, hsa-miR-654-3p, hsa-miR-654-5p, hsa-miR-655, hsa-miR-656, hsa-miR-657, hsa-miR-658, hsa-miR-659, hsa-miR-660, hsa-miR-661, hsa-miR-662, hsa-miR-663, hsa-miR-663b, hsa-miR-664, hsa-miR-664*, hsa-miR-665, hsa-miR-668, hsa-miR-671-3p, hsa-miR-671-5p, hsa-miR-675, hsa-miR-7, hsa-miR-708, hsa-miR-708*, hsa-miR-7-1*, hsa-miR-7-2*, hsa-miR-720, hsa-miR-744, hsa-miR-744*, hsa-miR-758, hsa-miR-760, hsa-miR-765, hsa-miR-766, hsa-miR-767-3p, hsa-miR-767-5p, hsa-miR-768-3p, hsa-miR-768-5p, hsa-miR-769-3p, hsa-miR-769-5p, hsa-miR-770-5p, hsa-miR-802, hsa-miR-873, hsa-miR-874, hsa-miR-875-3p, hsa-miR-875-5p, hsa-miR-876-3p, hsa-miR-876-5p, hsa-miR-877, hsa-miR-877*, hsa-miR-885-3p, hsa-miR-885-5p, hsa-miR-886-3p, hsa-miR-886-5p, hsa-miR-887, hsa-miR-888, hsa-miR-888*, hsa-miR-889, hsa-miR-890, hsa-miR-891a, hsa-miR-891b, hsa-miR-892a, hsa-miR-892b, hsa-miR-9, hsa-miR-9*, hsa-miR-920, hsa-miR-921, hsa-miR-922, hsa-miR-923, hsa-miR-924, hsa-miR-92a, hsa-miR-92a-1*, hsa-miR-92a-2*, hsa-miR-92b, hsa-miR-92b*, hsa-miR-93, hsa-miR-93*, hsa-miR-933, hsa-miR-934, hsa-miR-935, hsa-miR-936, hsa-miR-937, hsa-miR-938, hsa-miR-939, hsa-miR-940, hsa-miR-941, hsa-miR-942, hsa-miR-943, hsa-miR-944, hsa-miR-95, hsa-miR-96, hsa-miR-96*, hsa-miR-98, hsa-miR-99a, hsa-miR-99a*, hsa-miR-99b, and hsa-miR-99b*.

A miRNA inhibits the function of the mRNAs it targets and, as a result, inhibits expression of the polypeptides encoded by the mRNAs. Thus, blocking (partially or totally) the activity of the miRNA (e.g., silencing the miRNA) can effectively induce, or restore, expression of a polypeptide whose expression is inhibited (derepress the polypeptide). In one embodiment, derepression of polypeptides encoded by mRNA targets of a miRNA is accomplished by inhibiting the miRNA activity in cells through any one of a variety of methods. For example, blocking the activity of a miRNA can be accomplished by hybridization with a small interfering nucleic acid (e.g., antisense oligonucleotide, miRNA sponge, TuD RNA) that is complementary, or substantially complementary to, the miRNA, thereby blocking interaction of the miRNA with its target mRNA. As used herein, an small interfering nucleic acid that is substantially complementary to a miRNA is one that is capable of hybridizing with a miRNA, and blocking the miRNA's activity. In some embodiments, a small interfering nucleic acid that is substantially complementary to a miRNA is a small interfering nucleic acid that is complementary to the miRNA at all but 1, 2, 3, 4, 5, 6, 7, 8, 9, 10, 11, 12, 13, 14, 15, 16, 17, or 18 bases. In some embodiments, a small interfering nucleic acid sequence that is substantially complementary to a miRNA, or is a small interfering nucleic acid sequence that is complementary to the miRNA with at least one base.

A “miRNA Inhibitor” is an agent that blocks miRNA function, expression and/or processing. For instance, these molecules include but are not limited to microRNA specific antisense, microRNA sponges, tough decoy RNAs (TuD RNAs) and microRNA oligonucleotides (double-stranded, hairpin, short oligonucleotides) that inhibit miRNA interaction with a Drosha complex. MicroRNA inhibitors can be expressed in cells from a transgenes of a rAAV vector, as discussed above. MicroRNA sponges specifically inhibit miRNAs through a complementary heptameric seed sequence (Ebert, M. S. Nature Methods, Epub Aug. 12, 2007). In some embodiments, an entire family of miRNAs can be silenced using a single sponge sequence. TuD RNAs achieve efficient and long-term-suppression of specific miRNAs in mammalian cells (See, e.g., Takeshi Haraguchi, et al., Nucleic Acids Research, 2009, Vol. 37, No. 6 e43, the contents of which relating to TuD RNAs are incorporated herein by reference). Other methods for silencing miRNA function (derepression of miRNA targets) in cells will be apparent to one of ordinary skill in the art.

In some embodiments, the cloning capacity of the recombinant RNA vector may limit a desired coding sequence and may require the complete replacement of the virus's 4.8 kilobase genome. Large genes may, therefore, not be suitable for use in a standard recombinant AAV vector, in some cases. The skilled artisan will appreciate that options are available in the art for overcoming a limited coding capacity. For example, the AAV ITRs of two genomes can anneal to form head to tail concatamers, almost doubling the capacity of the vector. Insertion of splice sites allows for the removal of the ITRs from the transcript. Other options for overcoming a limited cloning capacity will be apparent to the skilled artisan.

Somatic Transgenic Animal Models Produced Using rAAV-Based Gene Transfer

The disclosure also relates to the production of somatic transgenic animal models of disease using recombinant Adeno-Associated Virus (rAAV) based methods. The methods are based, at least in part, on the observation that AAV serotypes and variants thereof mediate efficient and stable gene transfer in a tissue specific manner in adult animals. The rAAV elements (capsid, promoter, transgene products) are combined to achieve somatic transgenic animal models that express a stable transgene in a time and tissue specific manner. The somatic transgenic animal produced by the methods of the disclosure can serve as useful models of human disease, pathological state, and/or to characterize the effects of gene for which the function (e.g., tissue specific, disease role) is unknown or not fully understood. For example, an animal (e.g., mouse) can be infected at a distinct developmental stage (e.g., age) with a rAAV comprising a capsid having a specific tissue targeting capability (e.g., liver, heart, pancreas) and a transgene having a tissue specific promoter driving expression of a gene involved in disease. Upon infection, the rAAV infects distinct cells of the target tissue and produces the product of the transgene.

In some embodiments, the sequence of the coding region of a transgene is modified. The modification may alter the function of the product encoded by the transgene. The effect of the modification can then be studied in vivo by generating a somatic transgenic animal model using the methods disclosed herein. In some embodiments, modification of the sequence of coding region is a nonsense mutation that results in a fragment (e.g., a truncated version). In other cases, the modification is a mis sense mutation that results in an amino acid substitution. Other modifications are possible and will be apparent to the skilled artisan.

In some embodiments, the transgene causes a pathological state. A transgene that causes a pathological state is a gene whose product has a role in a disease or disorder (e.g., causes the disease or disorder, makes the animal susceptible to the disease or disorder) and/or may induce the disease or disorder in the animal. The animal can then be observed to evaluate any number of aspects of the disease (e.g., progression, response to treatment, etc.). These examples are not meant to be limiting, other aspects and examples are disclosed herein and described in more detail below.

The disclosure in some aspects, provide methods for producing somatic transgenic animal models through the targeted destruction of specific cell types. For example, models of type 1 diabetes can be produced by the targeted destruction of pancreatic Beta-islets. In other examples, the targeted destruction of specific cell types can be used to evaluate the role of specific cell types on human disease. In this regard, transgenes that encode cellular toxins (e.g., diphtheria toxin A (DTA)) or pro-apoptotic genes (NTR, Box, etc.) can be useful as transgenes for functional ablation of specific cell types. Other exemplary transgenes, whose products kill cells are embraced by the methods disclosed herein and will be apparent to one of ordinary skill in the art.

The disclosure, in some aspects, provides methods for producing somatic transgenic animal models to study the long-term effects of over-expression or knockdown of genes. The long term over expression or knockdown (e.g., by shRNA, miRNA, miRNA inhibitor, etc.) of genes in specific target tissues can disturb normal metabolic balance and establish a pathological state, thereby producing an animal model of a disease, such as, for example, cancer. The disclosure in some aspects, provides methods for producing somatic transgenic animal models to study the long-term effects of over-expression or knockdown of gene of potential oncogenes and other genes to study tumorigenesis and gene function in the targeted tissues. Useful transgene products include proteins that are known to be associated with cancer and small interfering nucleic acids inhibiting the expression of such proteins. Other suitable transgenes may be readily selected by one of skill in the art provided that they are useful for creating animal models of tissue-specific pathological state and/or disease.

### Recombinant AAV Administration Methods

The rAAVs may be delivered to a subject in compositions according to any appropriate methods known in the art. The rAAV, preferably suspended in a physiologically compatible carrier (e.g., in a composition), may be administered to a subject, e.g., host animal, such as a human, mouse, rat, cat, dog, sheep, rabbit, horse, cow, goat, pig, guinea pig, hamster, chicken, turkey, or a non-human primate (e.g., Macaque). In some embodiments a host animal does not include a human.

Delivery of the rAAVs to a mammalian subject may be by, for example, intramuscular injection or by administration into the bloodstream of the mammalian subject. Administration into the bloodstream may be by injection into a vein, an artery, or any other vascular conduit. In some embodiments, the rAAVs are administered into the bloodstream by way of isolated limb perfusion, a technique well known in the surgical arts, the method essentially enabling the artisan to isolate a limb from the systemic circulation prior to administration of the rAAV virions. A variant of the isolated limb perfusion technique, described in U.S. Pat. No. 6,177,403, can also be employed by the skilled artisan to administer the virions into the vasculature of an isolated limb to potentially enhance transduction into muscle cells or tissue. Moreover, in certain instances, it may be desirable to deliver the virions to the CNS of a subject. By “CNS” is meant all cells and tissue of the brain and spinal cord of a vertebrate. Thus, the term includes, but is not limited to, neuronal cells, glial cells, astrocytes, cereobrospinal fluid (CSF), interstitial spaces, bone, cartilage and the like. Recombinant AAVs may be delivered directly to the CNS or brain by injection into, e.g., the ventricular region, as well as to the striatum (e.g., the caudate nucleus or putamen of the striatum), spinal cord and neuromuscular junction, or cerebellar lobule, with a needle, catheter or related device, using neurosurgical techniques known in the art, such as by stereotactic injection (see, e.g., Stein et al., J Virol 73:3424-3429, 1999; Davidson et al., PNAS 97:3428-3432, 2000; Davidson et al., Nat. Genet. 3:219-223, 1993; and Alisky and Davidson, Hum. Gene Ther. 11:2315-2329, 2000).

The compositions of the disclosure may comprise an rAAV alone, or in combination with one or more other viruses (e.g., a second rAAV encoding having one or more different transgenes). In some embodiments, a composition comprises 1, 2, 3, 4, 5, 6, 7, 8, 9, 10, or more different rAAVs each having one or more different transgenes.

Suitable carriers may be readily selected by one of skill in the art in view of the indication for which the rAAV is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The selection of the carrier is not a limitation of the present disclosure.

Optionally, the compositions of the disclosure may contain, in addition to the rAAV and carrier(s), other conventional pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

The rAAVs are administered in sufficient amounts to transfect the cells of a desired tissue and to provide sufficient levels of gene transfer and expression without undue adverse effects. Conventional and pharmaceutically acceptable routes of administration include, but are not limited to, direct delivery to the selected organ (e.g., intraportal delivery to the liver), oral, inhalation (including intranasal and intratracheal delivery), intraocular, intravenous, intramuscular, subcutaneous, intradermal, intratumoral, and other parental routes of administration. Routes of administration may be combined, if desired.

The dose of rAAV virions required to achieve a particular “therapeutic effect,” e.g., the units of dose in genome copies/per kilogram of body weight (GC/kg), will vary based on several factors including, but not limited to: the route of rAAV virion administration, the level of gene or RNA expression required to achieve a therapeutic effect, the specific disease or disorder being treated, and the stability of the gene or RNA product. One of skill in the art can readily determine a rAAV virion dose range to treat a patient having a particular disease or disorder based on the aforementioned factors, as well as other factors that are well known in the art.

An effective amount of an rAAV is an amount sufficient to target infect an animal, target a desired tissue. In some embodiments, an effective amount of an rAAV is an amount sufficient to produce a stable somatic transgenic animal model. The effective amount will depend primarily on factors such as the species, age, weight, health of the subject, and the tissue to be targeted, and may thus vary between animals or tissues. For example, an effective amount of the rAAV is generally in the range of from about 1 ml to about 100 ml of solution containing from about 10⁹ to 10¹⁶ genome copies. In some embodiments the rAAV is administered at a dose of 10¹⁰, 10¹¹, 10¹², 10¹³, 10¹⁴, or 10¹⁵ genome copies per subject. In some embodiments the rAAV is administered at a dose of 10¹⁰, 10¹¹, 10¹², 10¹³, or 10¹⁴ genome copies per kg. In some cases, a dosage between about 10¹¹ to 10¹² rAAV genome copies is appropriate. In certain embodiments, 10¹² rAAV genome copies is effective to target heart, liver, and pancreas tissues. In some cases, stable transgenic animals are produced by multiple doses of an rAAV.

In some embodiments, rAAV compositions are formulated to reduce aggregation of AAV particles in the composition, particularly where high rAAV concentrations are present (e.g., ˜10¹³ GC/ml or more). Methods for reducing aggregation of rAAVs are well-known in the art and, include, for example, addition of surfactants, pH adjustment, salt concentration adjustment, etc. (See, e.g., Wright F R, et al., Molecular Therapy (2005) 12, 171-178, the contents of which are incorporated herein by reference.)

Formulation of pharmaceutically-acceptable excipients and carrier solutions is well-known to those of skill in the art, as is the development of suitable dosing and treatment regimens for using the particular compositions described herein in a variety of treatment regimens.

Typically, these formulations may contain at least about 0.1% of the active compound or more, although the percentage of the active ingredient(s) may, of course, be varied and may conveniently be between about 1 or 2% and about 70% or 80% or more of the weight or volume of the total formulation. Naturally, the amount of active compound in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In certain circumstances it will be desirable to deliver the rAAV-based therapeutic constructs in suitably formulated pharmaceutical compositions disclosed herein either subcutaneously, intraopancreatically, intranasally, parenterally, intravenously, intramuscularly, intrathecally, or orally, intraperitoneally, or by inhalation. In some embodiments, the administration modalities as described in U.S. Pat. Nos. 5,543,158; 5,641,515 and 5,399,363 (each specifically incorporated herein by reference in its entirety) may be used to deliver rAAVs. In some embodiments, a preferred mode of administration is by portal vein injection.

The pharmaceutical forms suitable for injectable use include sterile aqueous solutions or dispersions and sterile powders for the extemporaneous preparation of sterile injectable solutions or dispersions. Dispersions may also be prepared in glycerol, liquid polyethylene glycols, and mixtures thereof and in oils. Under ordinary conditions of storage and use, these preparations contain a preservative to prevent the growth of microorganisms. In many cases the form is sterile and fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the host. The person responsible for administration will, in any event, determine the appropriate dose for the individual host.

Sterile injectable solutions are prepared by incorporating the active rAAV in the required amount in the appropriate solvent with various other ingredients enumerated herein, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The rAAV compositions disclosed herein may also be formulated in a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. The phrase “pharmaceutically-acceptable” refers to molecular entities and compositions that do not produce an allergic or similar untoward reaction when administered to a host.

Delivery vehicles such as liposomes, nanocapsules, microparticles, microspheres, lipid particles, vesicles, and the like, may be used for the introduction of the compositions of the present disclosure into suitable host cells. In particular, the rAAV vector delivered trangenes may be formulated for delivery either encapsulated in a lipid particle, a liposome, a vesicle, a nanosphere, or a nanoparticle or the like.

Such formulations may be preferred for the introduction of pharmaceutically acceptable formulations of the nucleic acids or the rAAV constructs disclosed herein. The formation and use of liposomes is generally known to those of skill in the art. Recently, liposomes were developed with improved serum stability and circulation half-times (U.S. Pat. No. 5,741,516). Further, various methods of liposome and liposome like preparations as potential drug carriers have been described (U.S. Pat. Nos. 5,567,434; 5,552,157; 5,565,213; 5,738,868 and 5,795,587).

Liposomes have been used successfully with a number of cell types that are normally resistant to transfection by other procedures. In addition, liposomes are free of the DNA length constraints that are typical of viral-based delivery systems. Liposomes have been used effectively to introduce genes, drugs, radiotherapeutic agents, viruses, transcription factors and allosteric effectors into a variety of cultured cell lines and animals. In addition, several successful clinical trials examining the effectiveness of liposome-mediated drug delivery have been completed.

Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 μm. Sonication of MLVs results in the formation of small unilamellar vesicles (SUVs) with diameters in the range of 200 to 500.ANG., containing an aqueous solution in the core.

Alternatively, nanocapsule formulations of the rAAV may be used. Nanocapsules can generally entrap substances in a stable and reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use.

In addition to the methods of delivery described above, the following techniques are also contemplated as alternative methods of delivering the rAAV compositions to a host. Sonophoresis (i.e., ultrasound) has been used and described in U.S. Pat. No. 5,656,016 as a device for enhancing the rate and efficacy of drug permeation into and through the circulatory system. Other drug delivery alternatives contemplated are intraosseous injection (U.S. Pat. No. 5,779,708), microchip devices (U.S. Pat. No. 5,797,898), ophthalmic formulations (Bourlais et al., 1998), transdermal matrices (U.S. Pat. Nos. 5,770,219 and 5,783,208) and feedback-controlled delivery (U.S. Pat. No. 5,697,899).

### Kits and Related Compositions

The agents described herein may, in some embodiments, be assembled into pharmaceutical or diagnostic or research kits to facilitate their use in therapeutic, diagnostic or research applications. A kit may include one or more containers housing the components of the disclosure and instructions for use. Specifically, such kits may include one or more agents described herein, along with instructions describing the intended application and the proper use of these agents. In certain embodiments agents in a kit may be in a pharmaceutical formulation and dosage suitable for a particular application and for a method of administration of the agents. Kits for research purposes may contain the components in appropriate concentrations or quantities for running various experiments.

The kit may be designed to facilitate use of the methods described herein by researchers and can take many forms. Each of the compositions of the kit, where applicable, may be provided in liquid form (e.g., in solution), or in solid form, (e.g., a dry powder). In certain cases, some of the compositions may be constitutable or otherwise proces sable (e.g., to an active form), for example, by the addition of a suitable solvent or other species (for example, water or a cell culture medium), which may or may not be provided with the kit. As used herein, “instructions” can define a component of instruction and/or promotion, and typically involve written instructions on or associated with packaging of the disclosure. Instructions also can include any oral or electronic instructions provided in any manner such that a user will clearly recognize that the instructions are to be associated with the kit, for example, audiovisual (e.g., videotape, DVD, etc.), Internet, and/or web-based communications, etc. The written instructions may be in a form prescribed by a governmental agency regulating the manufacture, use or sale of pharmaceuticals or biological products, which instructions can also reflects approval by the agency of manufacture, use or sale for animal administration.

The kit may contain any one or more of the components described herein in one or more containers. As an example, in one embodiment, the kit may include instructions for mixing one or more components of the kit and/or isolating and mixing a sample and applying to a subject. The kit may include a container housing agents described herein. The agents may be in the form of a liquid, gel or solid (powder). The agents may be prepared sterilely, packaged in syringe and shipped refrigerated. Alternatively it may be housed in a vial or other container for storage. A second container may have other agents prepared sterilely. Alternatively the kit may include the active agents premixed and shipped in a syringe, vial, tube, or other container. The kit may have one or more or all of the components required to administer the agents to an animal, such as a syringe, topical application devices, or iv needle tubing and bag, particularly in the case of the kits for producing specific somatic animal models.

The kit may have a variety of forms, such as a blister pouch, a shrink wrapped pouch, a vacuum sealable pouch, a sealable thermoformed tray, or a similar pouch or tray form, with the accessories loosely packed within the pouch, one or more tubes, containers, a box or a bag. The kit may be sterilized after the accessories are added, thereby allowing the individual accessories in the container to be otherwise unwrapped. The kits can be sterilized using any appropriate sterilization techniques, such as radiation sterilization, heat sterilization, or other sterilization methods known in the art. The kit may also include other components, depending on the specific application, for example, containers, cell media, salts, buffers, reagents, syringes, needles, a fabric, such as gauze, for applying or removing a disinfecting agent, disposable gloves, a support for the agents prior to administration etc.

The instructions included within the kit may involve methods for detecting a latent AAV in a cell. In addition, kits of the disclosure may include, instructions, a negative and/or positive control, containers, diluents and buffers for the sample, sample preparation tubes and a printed or electronic table of reference AAV sequence for sequence comparisons.

### EXAMPLES

### Example 1: Isolation of Transcriptionally Active, Novel AAV Capsid Sequences with Desired Tissue Tropisms and Properties from Human Tissues

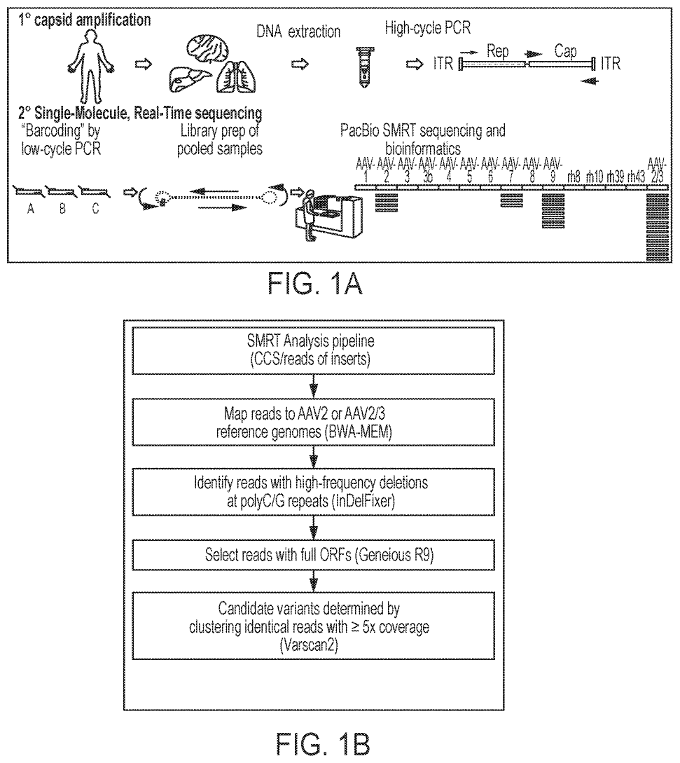

This example describes novel AAV capsid sequences isolated by the following steps: 1) PCR amplification of wtAAV genomes present in normal and diseased human tissues; 2) high-throughput single-molecule, real-time (SMRT) sequencing of PCR amplicon libraries; 3) variant identification/profiling by bioinformatic analyses; and 4) the selection of high-confidence ORFs that can be translated into full length capsid proteins. Schematic depictions of workflows used in this example are shown in FIGS. 1A-1B.

This approach exploits the natural pool of genomic diversity observed among viral genomes isolated from both normal and tumor tissues. Conceptually, in vivo tissues act as natural incubators for viral genomic diversity through selective pressure and/or immune evasion. Thus, the discovery of inter- and intra-tissue variability, as well as inter-patient diversity benefit from methods that are able to profile the full spectrum of AAV variants found among tissues and organs of human origin.

PCR Amplification of AAV Genomes from Human Tissues.

To isolate diverse AAV variants with the potential for identifying new serotypes with unique tropisms, 844 human surgical specimens from 455 patients were collected from West China Hospital, Sichuan University, Chengdu, China. These tissues encompass a wide-range of tissue/organ types, as well as various tumors types (Table 1). In particular, AAV variants were identified from nine normal liver tissues, 7 liver tumors, four enlarged prostate tissues, two normal lung tissues, one pancreatic tumor tissue, one breast cancer tissue, one normal breast tissue, one gastric cancer tissue, one normal gastric tissue, one brain tissue and one glioma sample.

Total genomic DNA was extracted from human tissues and subjected to PCR amplification of AAV capsid sequence. PCR primers used in this example are described in Table 2. Briefly, either panAAV primers for the amplification of 4.1-kb AAV rep-cap sequence (e.g., RepF318, AV2cas), or panAAV primers for amplification of 2.3-kb AAV cap sequence (e.g., CapF, CapR) were used for PCR.

TABLE 1
Clinical specimens for wtAAV genome amplification

| | Tissue quantity | |
|---|---|---|
| Organ | Normal tissue | Tumor tissue |
| Liver | 100 | 101 |
| Brain | 4 | 50 |
| Gastric | 37 | 37 |
| Lung | 100 | 100 |
| Breast | 52 | 57 |
| Pancreatic | NA | 45 |
| Rectal | 50 | 50 |
| Prostate | 34 | NA |
| Urologic | 3 | 12 |
| Cervical | 2 | 10 |
| Sum | 378 | 466 |

TABLE 2
PCR primer sequences

| Primer | Sequence (5′-3′) | SEQ ID NO: |
|---|---|---|
| RepF318 | GCCATGCCGGGGTTCTACGAGAT | 872 |
| AV2cas | ACAGGAGACCAAAGTTCAACTGAAACGA | 873 |
| CapF | GACTGCATCTTTGAACAATAAATGA | 874 |
| CapR | GAAACGAATTAACCGGTTTATTGATTAA | 875 |

### High-Throughput Sequencing of AAV PCR Products and Bioinformatics Analysis

AAV PCR products were subjected to high-throughput single-molecule, real-time (SMRT) sequencing. This approach removes the need to perform viral genome reconstruction and chimera prediction from aligned short-read fragments obtained from other conventional high-throughput genome sequencing methodologies.

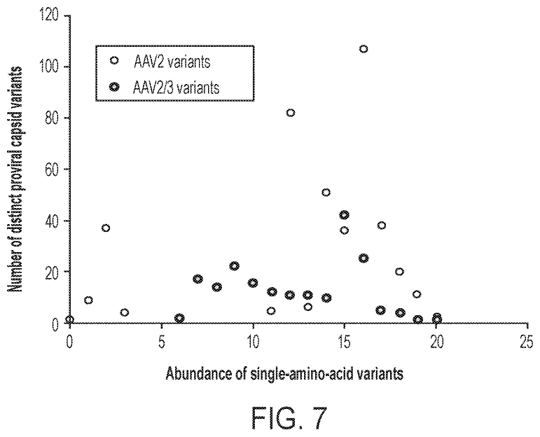

Using variant analysis pipelines developed from open source bioinformatic tools more than 600 previously undescribed, high-confidence AAV2, AAV2/3 hybrid, and AAV8 capsid sequence variants were identified. Specifically, 224 AAV8 variants (harboring 1 to 10 single amino acid variants); 425 AAV2 variants (harboring 1 to 20 single amino acid variants); and 194 AAV2/3 hybrid variants (harboring 10 to 50 single amino acid variants) were identified. Tables 3, 4 and 5 summarize the unique capsid protein variants. For purposes of comparison, wild-type AAV2, AAV3, and AAV8 capsid amino acid sequences are described in SEQ ID NOs: 869, 870, and 871, respectively. FIG. 7 is a scatter plot displaying the distribution of distinct AAV2 capsid variants and AAV2/3 variants harboring one or more single-amino-acid variants.

TABLE 3
Unique AAV2 and AAV2/3 hybrid variants (amino acid sequences)
identified by SMRT sequencing and bioinformatics analyses.

| | | | | | Total |
|---|---|---|---|---|---|
| Unique AAV2 variants | | | Unique | | unique |
| | Patient | Size of | variants | | variants |
| Sample Source | No. | DNA (kb) | (a.a.) | SEQ ID NOs: | (a.a.) |
| Liver | 7927N | 2.3 kb | 85 | 325-409 | 409 |
| Liver Tumor | 37HCC | (cap) | 3 | 322-324 | |
| Breast | 18B | | 26 | 118-143 | |
| Breast cancer | 19B | | 21 | 211-231 | |
| Lung | 18L | | 55 | 144-198 | |
| Prostate | 5 | | 24 | 1-24 | |
| | 17 | | 12 | 106-117 | |
| | 18 | | 12 | 199-210 | |
| | 27 | | 90 | 232-321 | |
| Pancreatic | 10 | | 81 | 25-105 | |
| cancer | | | | | |
| Liver | 1178N | 4.1 kb | 4 | 410-414; | 16 |
| | | (rep + cap) | | 837-840 | |
| | 9955N | | 3 | 429-434; | |
| | | | | 850-852 | |
| Liver tumor | 9955C | | 9 | 415-428; | |
| | | | | 841-849 | |
| | | | | | Total |
| Unique AAV2/3 variants | | | Unique | | unique |
| | Patient | variants | variants | | variants |
| Sample Source | No. | (a.a.) | (a.a.) | | (a.a.) |
| Liver | 42 | 2.3 kb | 6 | 512-517 | 194 |
| | 74 | (cap) | 11 | 543-553 | |
| Liver Tumor | 37HCC | | 6 | 506-511 | |
| | 65 | | 4 | 539-542 | |
| | 7449C | | 15 | 554-568 | |
| Breast | 18B | | 23 | 435-457 | |
| Breast cancer | 19B | | 44 | 462-505 | |
| Prostate | 5 | | 60 | 569-628 | |
| | 17 | | | | |
| | 18 | | 4 | 458-461 | |
| Gastric cancer | 17G | | N/A (420 | — | |
| | | | in DNA) | | |
| Gastric | 50G | | 21 | 518-538 | |

DNA sequences are provided for 4.1 kb libraries.

TABLE 4
Unique AAV8 variants (amino acid sequences) identified by SMRT
sequencing and bioinformatics analyses.

| | | | | | Total |
|---|---|---|---|---|---|
| | | | Unique | | unique |
| Sample | | Size of | variants | | variants |
| Source | Patient No. | DNA(kb) | (a.a.) | SEQ ID NOs: | (a.a.) |
| Liver | 0067N | 2.3kb(cap) | 12 | 647-658 | 208 |
| | 3522N | | 73 | 674-746 | |
| | 5110N | | 3 | 747-749 | |
| | 7427N | | 6 | 750-755 | |
| Liver Tumor | 0067C | | 9 | 638-646 | |
| | 7803C | | 9 | 756-764 | |
| | 8818C | | 63 | 765-827 | |
| Brain | G5 | | 9 | 828-836 | |
| Glioma | 2236 | | 14 | 659-672 | |
| Lung | 24 | | 10 | 629-637; 673 | |

TABLE 5
Additional AAV8 variant capsid proteins

| AAV8 Variant Name | SEQ ID NO: |
|---|---|
| B1 | 853 |
| B2 | 854 |
| B3 | 855 |
| B4 | 856 |
| B12 | 857 |
| B18 | 858 |
| B24 | 859 |
| B41 | 860 |
| B44 | 861 |
| B45 | 862 |
| B46 | 863 |
| B60 | 864 |
| B61 | 865 |
| B62 | 866 |
| B63 | 867 |
| B64 | 868 |

### Example 2: Identification of AAV8 Variants with Improved In Vivo Tropism

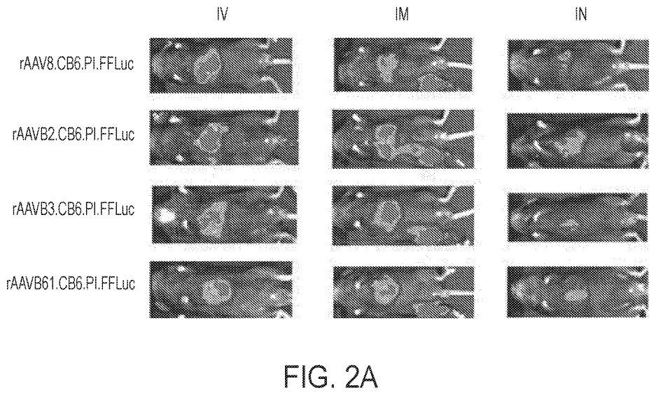

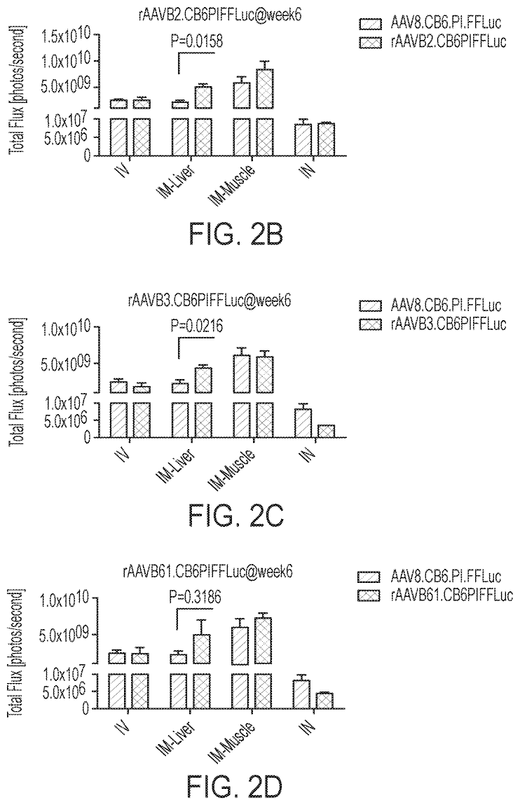

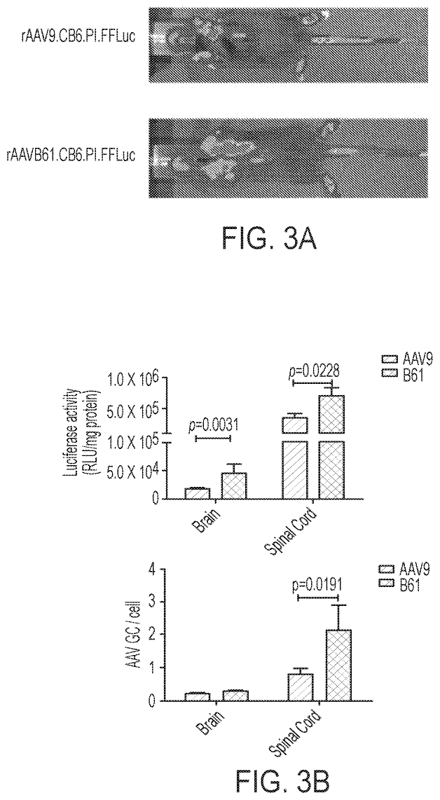

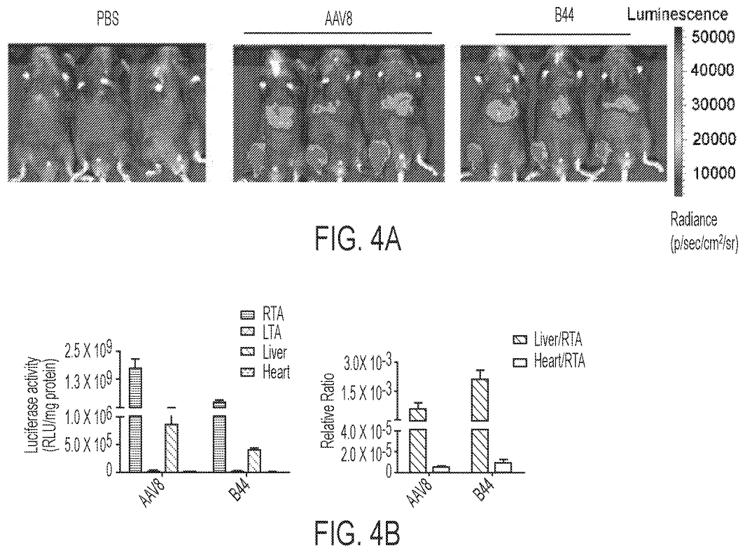

A subset of candidate AAV8 variants (e.g., B2, B3, B44 and B61) were cloned into AAV packaging vectors by standard molecular cloning methods, and packaged with luciferase reporter genes driven by the CB6 promoter. Produced vectors were injected into mice and in vivo levels of luciferase transgene expression were analyzed by whole animal imaging and quantification of relative luminescence. It was observed that the B2 (SEQ ID NO: 854) and B3 (SEQ ID NO: 855) variants have higher expression in liver after intramuscular injection (FIGS. 2A-2D), while after IV injection in neonatal mice, the B61 (SEQ ID NO: 865) variant has higher transduction efficiencies in brain and spinal cord compared to AAV9 (FIGS. 3A-3B). This is notable since wild-type AAV8 has been observed to cross the blood brain barrier less than AAV9. One AAV8 variant, B44 (SEQ ID NO: 861) has better ability transduced to liver after IM injection compared to AAV8 (FIGS. 4A-4B).

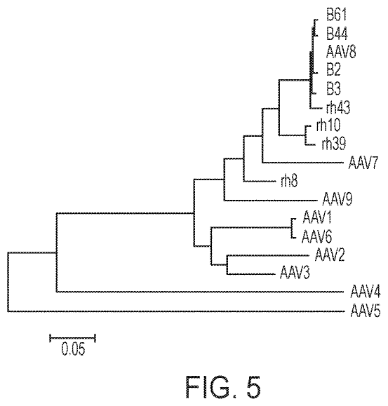

Phylogenic analysis was performed to compare AAV8 capsid variants B2, B3, B44, and B61 to other AAV serotypes. Briefly, amino acid sequences of AAV8 variants were aligned with other published AAV sequences using ClustalW and Phylogenetic trees were inferred using the Minimum Evolution method in MEGA6.06. Results of the bioinformatics analysis indicate that B2, B3, B44, and B61 sequences are related to Clade E [AAV8] capsid proteins. FIG. 5. Representative amino acid substitutions in AAV8 variants are shown in Table 6.

TABLE 6
Representative amino acid substitutions in AAV8 variants relative to
wild-type AAV8

| AAV Variant | Representative Substitutions (relative to wt AAV8) |
|---|---|
| B2 | E63G |
| B3 | K259R |
| B44 | L91Q, T234A, M374T |
| B61 | M374T, M561V |

### Example 3: In Vitro Assessment of rAAV Genome Packaging Efficiency and Initial Characterization of Candidate Capsid Variants

### Molecular Cloning of Packaging Plasmid Constructs Containing Selected AAV Capsid Variants

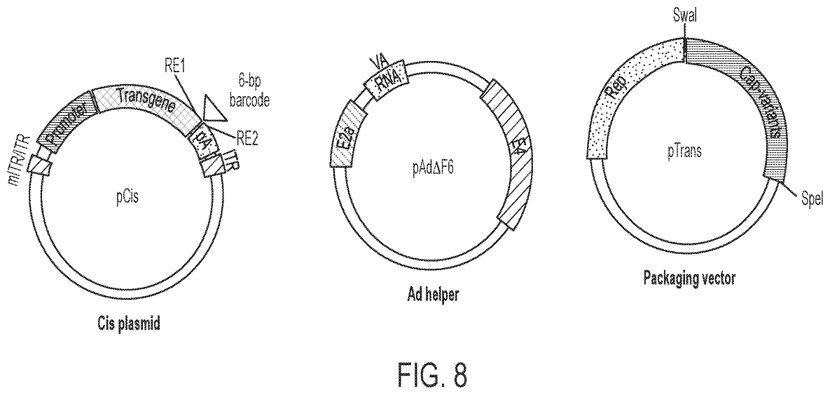

AAV2 and AAV2/3 hybrid capsid variants identified by SMRT sequencing are cloned into packaging plasmids by replacing the conventional viral capsid genes using standard molecular cloning strategies (e.g., site-directed mutagenesis of parental AAV2 or AAV2/3 capsid expression plasmids, PCR-based cloning and Gibson Assembly, or synthesized by outsourcing). FIG. 8 shows vector constructs to be used in multiplexed screening of discovered capsid variants. A summary of the proposed transgene cassettes to be used for various diagnostic strategies is shown in Table 7.

TABLE 7
Transgene cassettes for various diagnostic strategies

| Promoter | Transgene | Reporter/therapeutic gene analysis |
|---|---|---|
| CMV enhancer | EGFP | Tissue or cell-type specific |
| Chicken β-actin | | transduction efficiency |
| CMV enhancer | Luciferase | Whole-animal tropism profiling and |
| Chicken β-actin | | individual tissue quantification |
| Thyroxin | Factor IX | Liver-specific transduction of secreted |
| binding globulin | | factors. Pre-clinical testing |

### Multiplex Assessment of Packaging Efficiency by High-Throughput Small-Scale Vector Production and Titration for Vector Genomes

Quantification of vector genomes of rAAV in crude lysate is used to directly test rAAV variant packaging efficiency of both first-generation (single-strand AAV) and second-generation (self-complementary AAV) vectors directly following triple-transfection of HEK 293 packaging cells. This provides a streamlined alternative to performing the full workflow for small-scale vector production followed by silver staining and conventional PCR titration of vector genomes to assess virus quality for all discovered variants. Since this method can be scaled for performance in 96-well formats, it us used to quickly identify variants that produce high titer vectors.

### Serological Evaluation of Novel AAV Variants

Candidate variants with high packaging efficiency are screened for antibody cross-reactivity to current AAVs by standard means, such as capsid immunology assays to test novel rAAVs against serum from AAV-immunized rabbits. In addition, pooled human IgG (IVIG) neutralizing assays are performed for each candidate variant to determine the potential for pre-existing humoral immunity in the human population.

### Example 4: In Vivo Analyses of rAAV2 and rAAV2/3 Variants to Study Vector Transduction Biology, Prevalence of Pathotoxicity, Tissue/Organ Tropism, and Bio-Distribution Profiles

### Mouse Studies

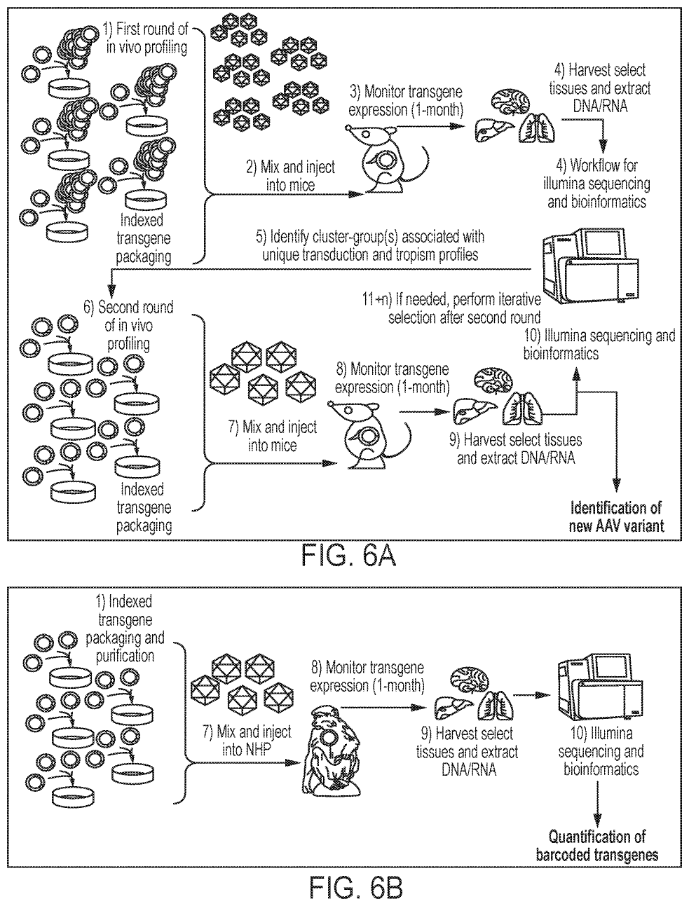

Candidate capsid variants are grouped based on tissue distribution, and prioritized by organs of interest. Groups of candidate variants are subjected to clustered-indexing (FIG. 6A), whereby multiple packaging plasmids expressing candidate capsid variants are mixed and expressed to package uniquely DNA barcoded transgenes by triple-transfection (e.g., F9 coagulation factor IX (F.IX), to assess liver targeting and expression efficacy of secreted factors; EGFP, to assess bio-distribution and the extent of tissue-specific transduction via organ/tissue sectioning and comparative immunofluorescence microscopy; or Luciferase (Luc), to assess the quality of CNS and liver transduction via live animal imaging

For studies that gauge the capacity of rAAV variants for liver-targeted transgene expression and secretion, rAAV constructs comprising the thyroxine-binding globulin (TGB), a liver specific promoter, are designed. For studies that profile whole-animal vector transduction, constructs comprising the CMV-enhancer, chicken β-actin promoter (CB6) regulatory cassette are designed.

Vectors encapsidating indexed transgenes are injected into adult and newborn mice by different routes of administration, and screened for secreted F.IX expression, EGFP expression, or Luc expression in 1-month longitudinal studies to profile AAV variant-mediated transgene expression. Routes of administration for the CNS/brain include peripheral intravascular (IV, to test transduction across the blood-brain barrier), intracerebroventricular (ICV), intraparenchymal, and intrathecal. Administration for retina is performed via subretinal injection. In some embodiments, IV injections also target the liver.

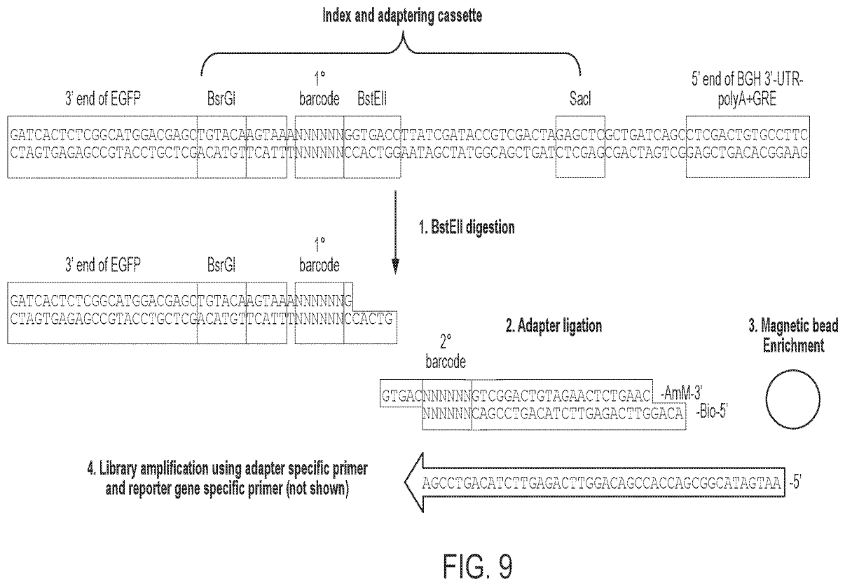

Animals that exhibit unique transgene expression compared to control animals (e.g., transgenes delivered by AAV2, AAV2/3, or AAV8) are sacrificed and harvested for organs. Individual organs are assayed for the presence and abundance of barcoded transgenes by conventional PCR amplification of bulk DNA extracts or cDNA libraries containing transgene message, followed by Illumina sequencing to trace barcoded transgenes enriched in each tissue. FIG. 9 outlines the general design strategy for transgene indexing. The abundance and tissue/organ distribution of detectable barcoded transgenes reflects candidate rAAV variant tropism and transduction efficacy of each group. Highly efficacious candidate groups with desirable vector properties are selected. Individual candidate variants from selected groups are used to package barcoded transgenes for a second round of screening for the purpose of identifying individual, highly efficacious variants. Clustered-indexing can be carried out iteratively in multiple rounds of hierarchical selection to reduce the workload.

### Non-Human Primate (NHP) Studies

Candidate rAAV variants are screened for bio-distribution in non-human primates by a modality similar to the clustered-indexing methodology outlined for mouse studies (FIG. 6B). The transduction efficiencies to target organs via different routes of administration are re-assessed in NHPs to validate rAAV variant profiles observed in precursor mouse studies.

Immunogenicity, prevalence of neutralization antibody in human populations, capacity for genotoxicity, and general aspects of pathogenicity are gauged alongside primary assessments, for example histopathology of multiple tissues and organs to scrutinize T-cell or neutrophil infiltrates, monitoring hepatotoxicity by ALT/AST activity, and analyzing inflammation by examination of histological sections, to determine transduction profiles in non-human primate (NHP) animals.

### Example 5: Isolation of Novel AAV Capsid Sequences

Additional AAV capsid sequences were isolated. Using variant analysis pipelines developed from bioinformatic tools, an additional 263 previously undescribed, high-confidence AAV2 and AAV2/3 hybrid capsid sequence variants were identified. For purposes of comparison, wild-type AAV2 and AAV3 capsid amino acid sequences are described in SEQ ID NOs: 869 and 870, respectively.

TABLE 8
Additional unique AAV2 and AAV2/3 hybrid variants (amino acid
sequences) identified by SMRT sequencing and bioinformatics
analyses.

| | | | | Total |
|---|---|---|---|---|
| | | Unique | | unique |
| Unique AAV2 variants | Size of | variants | SEQ ID NOs | variants |
| Sample Source | DNA (kb) | (a.a.) | (aa): | (a.a.) |
| Breast Cancer | 2.2 kb | 8 | 1726-1733 | 89 |
| Gastric Tumor | | 15 | 1734-1748 | |
| Glioma | | 2 | 1749-1750 | |
| Liver | | 25 | 1751-1775 | |
| Liver Tumor | | 36 | 1776-1811 | |
| Lung Tumor | | 3 | 1812-1814 | |
| | | | | Total |
| | | Unique | | unique |
| Unique AAV2/3 variants | Size of | variants | | variants |
| Sample Source | DNA (kb) | (a.a.) | | (a.a.) |
| Breast Cancer | 2.2 kb | 18 | 1815-1832 | 174 |
| Gastric | | 17 | 1833-1849 | |
| Liver | | 117 | 1850-1966 | |
| Liver Tumor | | 22 | 1967-1988 | |

The corresponding DNA sequences are provided for all libraries. The nucleic acid sequences for the AAV2 capsid variants correspond to SEQ ID NOs: 1989-2077. The nucleic acid sequences for the AAV2/3 capsid variants correspond to SEQ ID NOs: 2078-2251.

### Example 6: AAVv66

Recombinant adeno-associated viruses (rAAVs) have recently gained a lot of attention within the human gene therapy field, as safe and reliable gene delivery vehicles. AAV2 is currently most commonly used in preclinical and clinical studies. However, AAV2-based drug Luxturna is the only FDA approved virus-based biotherapeutic, so it is critical to improve the pharmaceutical properties of AAV.

AAV2 is known to be a “poor producer” for vector production and “underperforms” in many tissue- and cell-types. A virus variant with improved properties was isolated.

A variant named AAVv66 was identified as the most abundant pro-viral capsid variant in a clinical pancreatic neoplasm sample. The AAVv66 capsid harbors 13 residues that differ from AAV2. The variant exhibits favorable tropism in the CNS following subcranial injections. Furthermore, AAVv66 demonstrates better packaging efficiencies than prototypical AAV2. Using differential scanning fluorimetry (DSF), it was observed that the melting temperature of AAVv66 is ˜6° C. higher than AAV2 across a pH range spanning pH4-pH7. Furthermore, DSF analysis shows that at pH4, AAVv66 expunges its vector DNA at higher temperatures than AAV2.

It was also observed that AAVv66 confers superior CNS transduction relative to AAV2. Cryo-EM structure at 2.9 Å resolution reveals structural differences between AAV2 and AAVv66 at the 3-fold protrusions and at the interface of the 5-fold axis of symmetry, indicating that residues at these positions confer improved stability and function for vector transduction.

### Example 7: AAVv66 Experiments

AAVs have recently attracted attention as effective and proven gene therapy vectors. The current class of AAV vectors confer stable, long-term gene expression, have a broad range of tissue tropisms, and exhibit relatively low pathogenicity. To date, three serotype capsids (AAV1, AAV2, and AAV9) have gained regulatory approval for commercial use in patients. Unfortunately, the current library of discovered and engineered AAV capsids falls short for certain clinical applications that require targeting of specific tissues or cell types. Furthermore, patients may have pre-existing immunity to the vector via neutralizing antibodies that would limit therapeutic efficacy. Additionally, certain capsids are known to be problematic under standard production schemes for generating high-yield titers needed to meet therapeutic doses. In response to these shortcomings, there is a need to discover and develop novel capsids that exhibit better vector yields, can escape innate immunity, and possess unique tropism profiles.

This example describes a capsid protein variant, AAVv66 (SEQ ID NO: 66) that was identified by high-throughput single molecule real-time (SMRT) sequencing and whose properties substantially differ from those of AAV2 despite high (98%) sequence similarity. First, AAVv66 exhibits better vector yield and is more thermostable than prototypical AAV2. Second, AAVv66 has a better spread of distribution within brain tissue when administrated by intracranial injections. Finally, AAVv66 is antigenically distinct from AAV2.

To better understand how AAVv66 differs from AAV2, cryogenic electron microscopy (cryo-EM) was performed to explore the structural and functional characteristics that define AAVv66. Our 2.5-Å resolution structure of the AAVv66 capsid reveals differences from the structure of AAV2 and provides insights into the functional properties the capsids. Together, these observations describe the mechanistic properties of AAVv66.

### Materials and Methods

### DNA Extraction

A pancreatic neoplasm sample was acquired from a 71-year-old female patient following tumorectomy and pathology of the tissue by frozen section examination and intraoperative frozen section diagnosis. The sample was stored in liquid nitrogen until DNA extraction. To avoid AAV DNA cross-contamination, DNA extraction, and PCR procedures were performed in a sterile UV-irradiated biosafety cabinet. All surfaces and equipment were sprayed with DNA-Exitus Plus (Applichem, Cat No: A7089) and wiped clean with milli-Q water after 15 minutes. Frozen tissues were then thawed at room temperature, quickly cut to about 25 mg of tissue with disposable scalpels and placed in a 2 mL tube. Extractions of DNA from tissues were performed using the QIAamp DNA Mini Kit (Qiagen, #51306) according to manufacturer's recommended procedures.

### SMRT Sequencing

Amplicon libraries were generated from genomic DNA by standard PCR procedures. To amplify AAV genomes, PCR was performed using Platinum™ PCR SuperMix High Fidelity (Invitrogen) with the following cycle conditions: 97° C. for 1 min, 46 cycles of 98° C. for 10 s, 60° C. for 15 s, and 68° C. for 2 min 30 s; and 68° C. for 10 min. Correctly-sized PCR products were gel-purified with PureLink™ PCR Purification Kit (Thermo Fisher) and used for a second round of 15-cycle PCR for barcoding. The primer pairs used were:

First Round Primers:
(SEQ ID NO: 2252)
CapF 5′-GACTGCATCTTTGAACAATAAATGA-3′
and
(SEQ ID NO: 2253)
CapR 5′-GAAACGAATTAACCGGTTTATTGATTAA-3′
Second Round Primers: EF 5′-
(SEQ ID NO: 2254)
CATCACTACGCTAGATGACTGCATCTTTGAACAATAAATGA-3′
and
(SEQ ID NO: 2255)
ER 5′-TAGTATATCGAGACTCGAAACGAATTAACCGGTTTATTG
ATTAA-3′

Amplicons representing the capsid variant ORFs were subjected to standard SMRT sequencing library generation. Sequencing was performed on the RSII platform. SMRT sequencing returned 17,727 DNA reads that mapped to the AAV2 Cap ORF using the BWA-MEM algorithm. To rule out artifactual sequences, reads were then filtered to exclude those that were less than 1,800 nt and more than 2,500 nt in length, and then filtered on the quality of reads (Phred score>30). This filtering reduced the reads to 14,500. Finally, reads were processed through InDelFixer to remove single nucleotide insertions and deletions that may result from error-prone PCR or sequencing error. In order to consider only unique capsid sequences and to rule out low-confidence variants, de novo assembly (Geneious R9) was performed on the filtered reads to cluster reads with 99% of sequence similarity. Only read clusters represented by at least 10 reads were considered unique DNA capsid sequences. DNA sequences were then translated to amino acid sequences to define the final list of unique AAV capsids.

Full AAV Cap ORFs from contemporary AAV serotypes (hu.2 used for AAV2/3) were obtained from NCBI and the predicted amino acid sequences were aligned using the MUSCLE algorithm, iterating until convergence was achieved. PhyML was then used to generate the phylogenetic tree using default parameters from within SeaView55 and then visualized via the Interactive Tree of Life online tool.

### Viral Vector Production

Viruses were produced using the triple-transfection method in HEK293 cells and purified by CsCl gradient centrifugation. All vectors described were packaged with either the self-complementary AAV vector expressing enhanced green fluorescence protein (scAAV-CB6-EGFP), single-strand vector expressing Firefly luciferase (ssAAV-CB6-Fluc), single-strand vector expressing secreted human alpha1-anti-trypsin (ssAAV-CB6-hA1AT), or single-strand vector expressing LacZ. All transgenes are driven by the CMV early enhancer/chicken β actin (CB6) ubiquitous promoter.

### Animals

Six- to eight-week-old male C57BL/6J mice (The Jackson Laboratory) were injected by IV, IM, or intracranial administration of test vectors. Mice subjected to IV injections were administered with vectors packaged with ssAAV-CB6-Fluc transgenes (1.0E11 vg/mouse), and mice were sacrificed 14 days post-injection. Mice subjected to IM injections of the TA muscle, were administered with vectors packaged with ssAAV-CB6-Fluc transgenes (4.0E10 vg/mouse), and mice were sacrificed 28-days post-injection. Every week, up until, and at time of sacrifice, animals were injected intraperitoneally with D-luciferin substrate and sedated with isoflurane and luciferase activity was quantified using the IVIS SpectrumCT imaging platform with 1 min exposures. Image acquisition was performed using Living Image software. Mice subjected to intrahippocampal injections were administered with vectors packaged with scAAV-CB6-Egfp transgenes (3.6E9 vg/mouse). Unilateral injections were performed in the right hemisphere using a stereotaxic frame (Stoelting Co. Wood Dale, Ill.), Hamilton Syringe (1207K95, Thomas Scientific), and Hamilton Needle (77602-06, Hamilton). The following relative coordinates were used for all intra-hippocampal injections: x: −1.5 mm, y: −2 mm, z: −2 mm.

### Immunostaining

Four-weeks post-injection, animals were transcardially perfused with 1× phosphate buffered saline (PBS), followed by 4% paraformaldehyde (PFA). Brains were extracted and subsequently fixed in 4% PFA overnight at 4° C. Brains were then immersed in 30% sucrose (prepared in 1×PBS), at 4° C., until equilibrated in sucrose mixture. Brains were embedded in a 1:2 OCT (Tissue Tek, Torrance, Calif.) and 30% sucrose mixture, and cryo-sectioned at 40 μm (Cryostar NX70, ThermoScientific, Waltham, Mass.). Sections were permeabilized in 0.5% TritonX-100 for 1 hr, blocked in 5% goat serum (10% normal goat serum, 50062Z, Life Technologies) for 1 hr, and then incubated in primary antibody (anti-NeuN, 1:1000, EMD Millipore MAB377; anti-Gfap, 1:500, EMD Millipore MAB360; anti-Olig2, 1:200, Abcam ab109186; anti-Ibal, 1:1000, Wako Chemicals NC9288364) overnight at 4° C. Sections were washed three times in 1×PBS and incubated in secondary antibody (anti-mouse, Invitrogen A32744; or anti-rabbit, Invitrogen A32740) for 1 hour at room temperature. Sections were washed three times in 1×PBS and mounted with Vectashield containing DAPI (Vector Laboratories, Burlingame, Calif.).

### Microscopy

Brain section images were acquired on a Leica SP8 Lightning High Resolution Confocal (Leica Microsystems, Wetzlar, Germany). Global brain images (10× tiled brain sections) and high-magnification images (63× region specific areas) were collected at the same intensity and exposure thresholds for each respective magnification. For high-magnification images, 40-50 z-stack steps were collected at a 0.29 z-size. Analysis was performed using Imaris 9.3 Software (Bitplane Inc., Zurich, Switzerland). Each image was 3D rendered and thresholds were manually established. To ensure consistency, non-biased 3D rendering of total sub-anatomical EGFP volumes colocalized with DAPI volumes and cell type-specific stains were used as proxies for cellular counts and the number of positively transduced cells. Percent quantifications of the different cell types within each ipsilateral sub-anatomical region was conducted, followed by percentage quantification of each cell type. Percent transduction was determined by normalizing colocalized EGFP volume to total volume of cell type-specific staining within each region. Per cell-specific stain, n=3 mice were analyzed. Statistical calculations for FIG. 2b was conducted in Prism 7 (GraphPad Software, Inc., San Diego, Calif.) and analysis was performed using Student's unpaired t-test.

### DSF Analysis

For capsid stability experiments, 5 μL SYPRO Orange 5000× (Thermo Fisher Scientific) was diluted in 495 μL PBS (Corning) to make a 50× stock. 45 μL of virus was mixed with 5 μL of 50×SYPRO Orange (final SYPRO Orange concentration was 5×). Fluorescence was quantified using a ViiA 7 real-time PCR instrument (Thermo Fisher Scientific) with the following parameters: samples were incubated at 25° C. for 2 min, followed by a temperature gradient (25° C. to 99° C., 0.4° C. per step and held at each step for 2 min). To monitor the fluorescence of the SYPRO Orange at each temperature step, the ROX filter was used with no passive reference. To investigate the effect of pH on the melting temperature of AAV vectors, 5 μL of the virus vectors, 5 μL of 50× SYBR Orange, and 40 μL of 0.6M acetate buffer pH-adjusted from pH7 to pH4 were mixed. Tm values reported in this study is defined as the max Dsignal/Dtemp detected between 25 and 95° C. To investigate vector genome release, the SYBRO Orange dye was switched to SYBR Gold (Thermo Fisher Scientific).

### Site-Directed Mutagenesis

To generate point mutations in the AAVv66 capsid ORF, the Q5 site-directed mutagenesis kit (New England Biolabs) and following pairs of mutagenesis primers were used:

TABLE 9

| Mutation | F. Primer (lowercase = mutated | R. Primer (SEQ ID NOs: |
|---|---|---|
| in AAVv66 | bases) (SEQ ID NOs: 2256-2268) | 2269-2281) |
| Q39K | AGAGCGGCATaagGACGACAGCA | GCGGGCTTTGGTGGTGGT |
| A151V | GCATTCTCCTgtgGAGCCAGACT | TCTACCGGCCTCTTTTTTCC |
| K447R | TTACTTGAGCagaACAAACGCTC | TACAGATACTGGTCGATC |
| A450T | CAAAACAAACactCCAAGCGGAAC | CTCAAGTAATACAGATACTGG |
| M457Q | AACCACCACGcagTCCAGGCTTC | CCGCTTGGAGCGTTTGTT |
| A492S | ATCAAAAACAtctGCGGATAAC | ACTCGCTGCTGGCGGTAA |
| | AACAACAGTG | |
| D499E | CAACAACAGTgaaTATTCGTGGAC | TTATCCGCAGCTGTTTTTG |
| Y533F | TGAAGAAAAAtttTTTCCTCAGA | TCGTCCTTGTGGCTGGCC |
| | GCGGGGTTC | |
| D546G | TGGAAAACAAggcTCGGGAAAAA | AAGATGAGAACCCCGCTC |
| G548E | ACAAGACTCGgagAAAACTAATGTG | TTTCCAAAGATGAGAACC |
| S585R | CAACCTCCAGagaGGCAACACAC | GTAGATACAGAACCATACTGCTC |
| T588R | GAGCGGCAACagaCAGGCAGCCA | TGGAGGTTGGTAGATACAGA |
| | | ACCATACTG |
| T593A | GGCAGCCACCgcaGATGTCAACA | TGTGTGTTGCCGCTCTGG |

### Cryo-EM

AAVv66 was prepared for cryo-EM on grids with a lacey carbon support film (01824G, Ted Pella, Inc.). First, the grids were washed with acetyl acetate and allowed to dry overnight. Next, the grids were glow discharged with 20 mA current with negative polarity for 60 sec in a PELCO easiGlow glow discharge unit. 3 μL of 1E13 vg/mL AAVv66-CB6-Egfp vector in buffer (5% sorbitol, 0.001% pluronic acid F68 in PBS) was placed onto the grids loaded on a Vitrobot Mark IV (ThermoFisher) cryo-EM plunging apparatus. The grids were blotted for 6 to 6.5 seconds with Whatman #1 filter paper at 10° C. and 95% relative humidity prior to rapid freezing in liquid ethane.

A data set consisting of 2,033 movies was collected using SerialEM on a Titan Krios electron microscope (FEI) operating at 300 kV and equipped with a Gatan Image Filter (GIF) and a K2 Summit direct electron detector (Gatan Inc.) using 0.5-2.2 μm underfocus. 50 frames per movie were collected, and 34 frames were used at 1.43 e-/Å2 per frame for a total dose of 48.62 e-/Å2 on the sample. Pixel size was 1.0588 Å on the sample. Movies were imported into cisTEM and were aligned with dose filtering and CTF parameters were determined. Next, a total of 52,874 particles were automatically picked within cisTEM (characteristic and maximum radius: 130 and 140 Å). We note that both particles encapsulating vector transgenes and the small percentage of empty capsids were used to determine the final structure. Within cisTEM, an initial reference for alignment was generated from all particles using the Ab initio 3D reconstruction function. This reference and all particles were iteratively refined using auto refine to obtain a 2.95-Å resolution map as determined from the FSC_part cutoff at 0.143. One round of per-particle CTF refinement in manual mode improved map resolution to 2.62 Å. Lastly, one round of beam tilt refinement and reconstruction improved map resolution to 2.46 Å. 3D classification did not improve the maps. The final map was B-factor sharpened by applying a B-factor of −32.92 Å2 using the PHENIX auto sharpen function.

Cryo-EM structure of AAV2 (PDB ID: 1LP3) was used as a starting model for structure refinement. Variant residues were modeled using PyMOL (The PyMOL Molecular Graphics System, Version 2.0 Schrödinger, LLC.). The resulting AAVv66 model, containing 60 copies of VP3, was refined using PHENIX59 against the cryo-EM map. Real-space simulated annealing and B-factor refinement in PHENIX resulted in a stereochemically optimal model. The refinement results are summarized in Table 10. The model was inspected, and figures were prepared using PyMOL.

Vectors were diluted to a concentration of ˜1.0E9 vg/mL for zeta potential analysis using the Zetasizer Nano ZS system (Malvern). 500 μL of sample was added into a universal dip cell (Malvern). Before measurement, the system was stabilized for 2 min. Three measurements were recorded for each sample.

TABLE 10
Cryo-EM data collection, refinement and
validation statistics

| | #1 name |
|---|---|
| | (EMDB-20630) |
| | (PDB 6U3Q) |
| Data collection and processing | |
| Magnification | 47,214 |
| Voltage (kV) | 300 |
| Electron exposure (e-/A²) | 48.62 |
| Defocus range (μm) | 0.4-5.0 |
| Pixel size (A) | 1.059 |
| Symmetry imposed | I |
| Initial particle images (no.) | 52,874 |
| Final particle images (no.) | 52,874 |
| Map resolution (A) | 2.46 |
| FSC threshold | 0.143 |
| Refinement | |
| Initial model used (PDB code) | 1LP3 |
| Model resolution (A) | 2.6 |
| FSC threshold | 0.5 |
| Map sharpening B factor (A²) | 32.92 |
| Model composition | |
| Non-hydrogen atoms | 248,280 |
| Protein residues | 31,140 |
| Ligands | 0 |
| B factors (A²) | |
| Protein | 86.48 |
| Ligand | 0 |
| r.m.s. deviations | |
| Bond lengths (A) | 0.009 |
| Bond angles (°) | 0.603 |
| Validation | |
| MolProbity score | 1.66 |
| Clashscore | 2.25 |
| Poor rotamers (%) | 4.15 |
| Ramachandran plot | |
| Favored (%) | 96.91 |
| Allowed (%) | 4.15 |
| Disallowed (%) | 0.00 |

### Immunological Studies

1.0E11 vg/mice of scAAV-CB6-Egfp were intramuscularly administrated into the left/right tibialis anterior of C57BL/6J mice. Four weeks later, 1.0E11 vg/mice of ssAAVv66-CB6-hA1AT or ssAAV2-CB6-hA1AT was delivered to the contralateral leg. Serum was collected at weeks 4, 5, 6, 7, and 8 by facial vein bleeds to assess neutralizing antibody titers and A1AT levels by ELISA.

Huh-7.5 (5.0E4 cells/well) were seeded onto a 96-well plate 24 hr prior to transduction at 37° C. Ad helper virus was then added at a multiplicity of infection (MOI) of 100:1 to the cell monolayer and incubated for at least an hour. Serial dilutions of serum and ssAAV2-LacZ or ssAAVv66-LacZ mixed solution were prepared in a V-bottom 96-well plate and incubated at 37° C. for 1 hr. The serum-AAV mixed solution was then added to cells and incubated at 37° C. for 24 hr. Cells were lysed and treated with beta-galactosidase substrate using the Galacto-Star One-Step Assay System (Invitrogen). Luminescence signal was detected by Synergy HT microplate reader (BioTek, Winooski, Vt.).

### A1AT ELISA

A 96-well plate was first coated with anti-A1AT antibody at 4° C. overnight, and wells were incubated with blocking buffer (1% non-fat milk and 0.05% Tween-20 in PBS buffer) for 1 hr at room temperature. 1/20, 1/200 and 1/2,000 serum dilutions were performed using sample buffer (0.05% Tween-20 in PBS buffer) in a 96-well plate along with positive control (100, 50, 25, 12.5, 6.25, and 3.125 ng/mL A1AT). After the plate was washed 3 times, sera were added into each well and incubated at 4° C. overnight. The plate was then washed 3 times and incubated with goat anti-trypsin-HRP antibody (1:5,500 dilution in sample buffer) for 2 hr. Before reacting with substrate, the plate was washed 6 times to remove all residual proteins. Lastly, ABTS substrates were added into wells and the signal was read by a Synergy HT microplate reader (BioTek).

### Identification of Novel AAV Variants in a Human Tissue Sample by Long-Read Sequencing

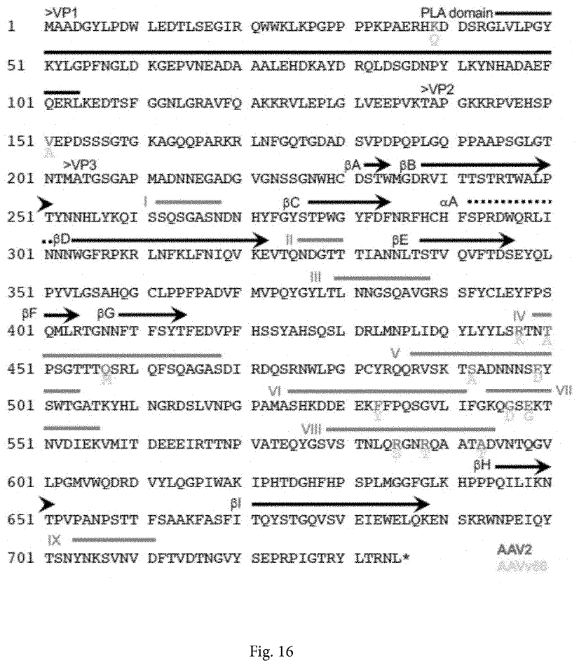

To identify novel full-length capsid sequences from human tissues, SMRT sequencing was performed to obtain long DNA reads that span the entire capsid open reading frame. This method can resolve sequences of long DNA fragments without the need for sequence assembly, which is necessary in short-read sequencing approaches. In this way, capsid diversity that is defined by both point mutations and recombination events can be assessed on individual intact molecules that span the entire capsid ORF. To explore AAV diversity, a single tissue was selected from about 800 human surgical samples. Using primers that flank the capsid ORF at conserved sequence across known serotypes, target PCR amplicons were produced for SMRT sequencing analysis. One capsid sequence that made up ˜45% of all sequences identified from the single tissue was isolated. This predominant capsid, named “variant 66” (AAVv66), exhibits closest homology to AAV2 (98% sequence similarity). It was observed that AAVv66 contains 13 amino acid residues that differ from AAV2 (FIG. 16): one within the VP1u region (K39Q), one within the VP2 domain (V151A), and eleven within VP3 (R447K, T450A, Q457M, S492A, E499D, F533Y, G546D, E548G, R585S, R588T and A593T). Notably, the unique amino acid residues within VP3 are all within or near variable regions VR-IV through VR-VIII.

The VP3 region of AAVv66 was compared with those of other contemporary AAV serotypes (AAV1-AAV9). The most notable differences occur at four positions (499, 533, 585, and 588), which are highly conserved among AAV serotypes (FIG. 16). At position 499, most serotypes harbor an asparagine, while AAVv66, AAV2, AAV4, and AAV9 have a negatively charged aspartic acid or glutamic acid. The highly conserved phenylalanine at position 533 is a tyrosine in AAVv66 (also, T533 in AAV5). Finally, unlike AAV2, which harbors positively charged arginine residues that define AAV2's capacity to bind heparan sulfate proteoglycans (HSPG) at positions 585 and 58823, AAVv66 contains 5585 and T588 (identical to AAV1, AAV3, AAV5, and AAV6).

AAVv66 Vector Production and Cell Infectivity Differ from Those of AAV2

The strong affinity of AAV2 for heparin and its resulting strong cell-surface association is proposed to lead to the virus' relatively poor packaging titers. The limited vector yield by AAV2 is thought to result from non-productive binding and re-infection of the packaging cells by vector particles during production. Vector production and cell infectivity of AAVv66 was compared with those of AAV2 and AAV3b. Of note, AAV3b is the closest distinct cousin to AAV2 (89% sequence similarity), but uses different electrostatic surface charges at the 3-fold protrusions to weakly bind heparin. This difference between AAV3b and AAV2 likely explains AAV3b's increased packaging titers that result from transduction of HEK producer cells.

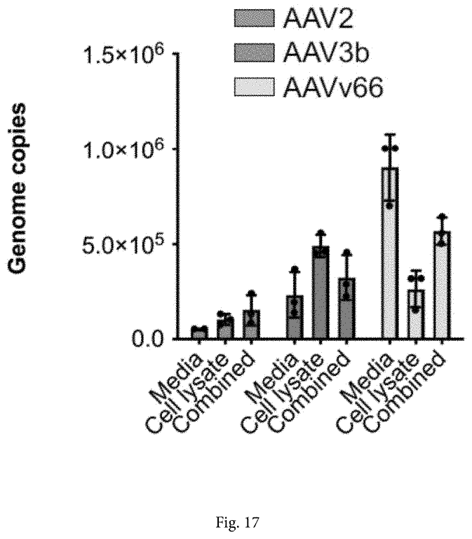

The packaging profiles of AAVv66 were compared with those of AAV2 and AAV3b by measuring the yields of encapsidated vector genomes in cell lysates. To this end, the AAVv66 capsid ORF was synthesized and cloned into a trans-plasmid expressing AAV2 Rep under the AAV2 p5 promoter (pAAV2/v66). Small-scale vector preparations of AAVv66, AAV2, and AAV3b were used to package a single-stranded vector consisting of the firefly luciferase transgene driven by the ubiquitous chicken-beta actin promoter (AAV-CB6-Fluc). Quantification of viral vector yields by crude lysate qPCR29 revealed that the yield of encapsidated, DNase-resistant genomes of AAVv66 vectors is ˜2.4-fold higher than AAV2 yields and is ˜30% higher than AAV3b yields (FIG. 17, “combined” samples).

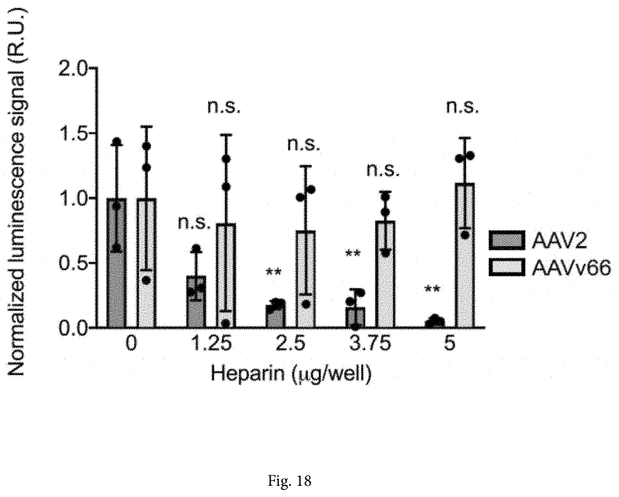

Whether the higher abundance of AAVv66 in crude lysate is due to non-productive binding of particles to the packaging cells, which would be manifested by a predominance of AAVv66 particles in the media rather than in the cell lysate fraction, was investigated next. PCR analysis revealed that encapsidated genomes of AAVv66 within the media are ˜3-fold more abundant than in cell lysates (FIG. 17). In contrast, very few AAV2 particles were detected in the media of packaging cells. To test whether AAVv66's ability to produce more DNase-resistant genomes is related to weak re-infectivity of packaging cells due to poor HSPG binding, a heparin competition assay was performed (FIG. 18). For this purpose, large-scale AAVv66 and AAV2 vectors, again packaging CB6-Fluc, were produced using the standard cesium chloride purification protocol. Transduction of AAVv66 is not affected by the presence of heparin, whereas 1.25 μg/well of heparin blocked AAV2 transduction by 50% and 5 μg/well of heparin completely abolished transduction. These results indicate that the improved production efficiency of AAVv66 is at least in part due to poor heparin binding.

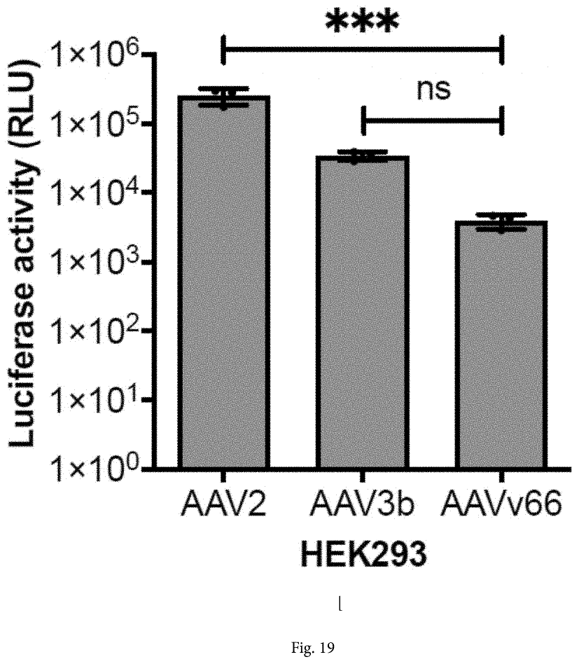

To determine whether AAVv66's lower affinity to heparin coincides with reduced cell transduction compared to AAV2, purified AAVv66, AAV2, and AAV3b vectors were used to infect HEK293 cells. Data indicate that AAV2 exhibits greater transduction than AAVv66 (˜65-fold) and AAV3b (˜7.5-fold) (FIG. 19). Vectorized AAVv66 proviral capsid sequence is able to efficiently transduce cells in vitro, but its vector production and cell infectivity properties are distinct from those of its closest serotype relative, AAV2.

AAVv66 Exhibits CNS Transduction that is Distinct from that of AAV2

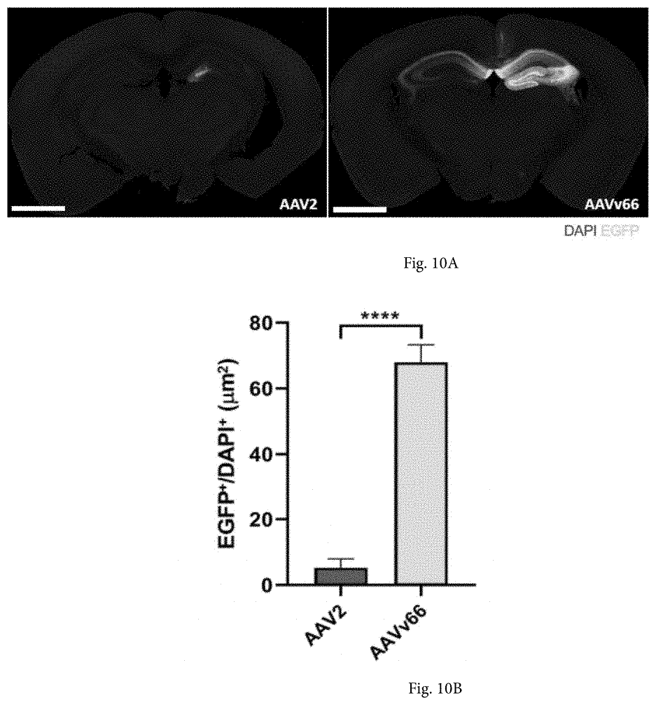

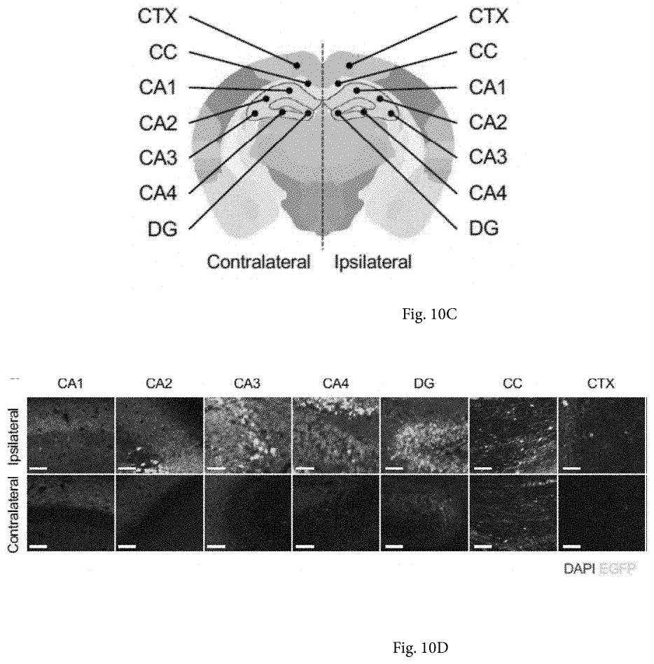

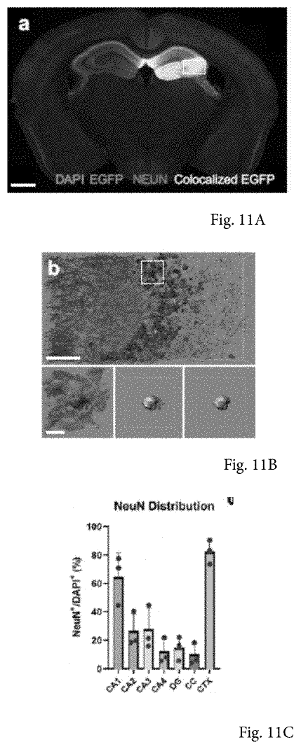

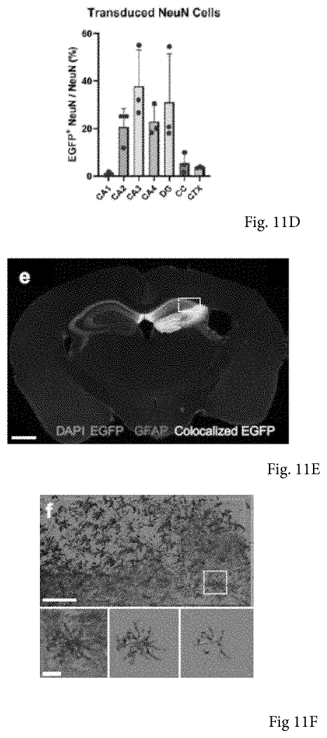

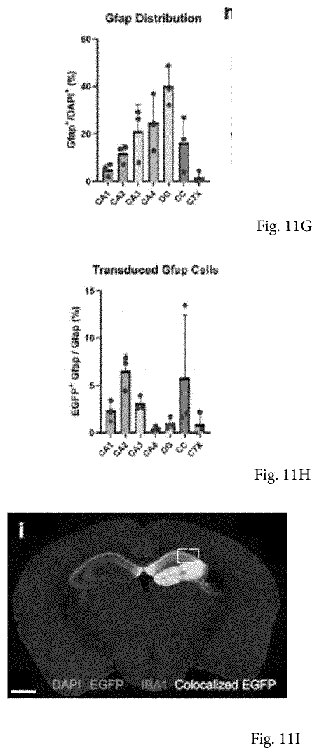

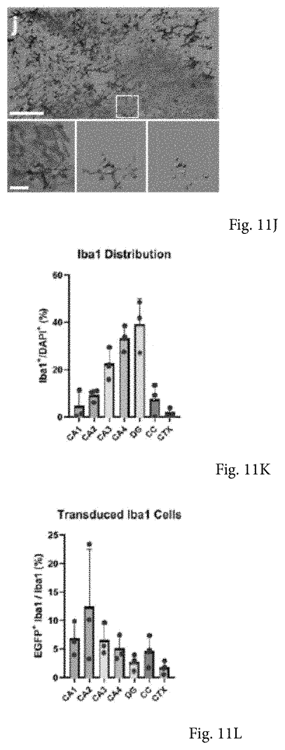

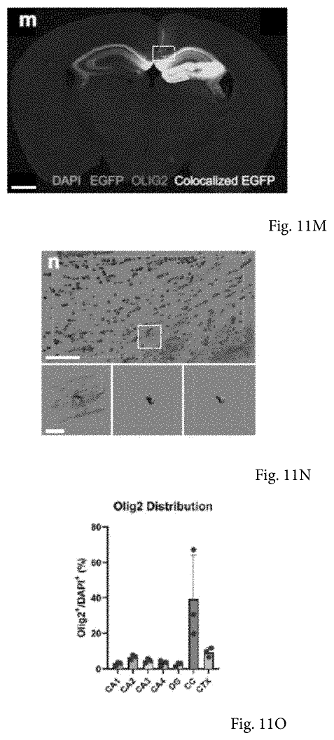

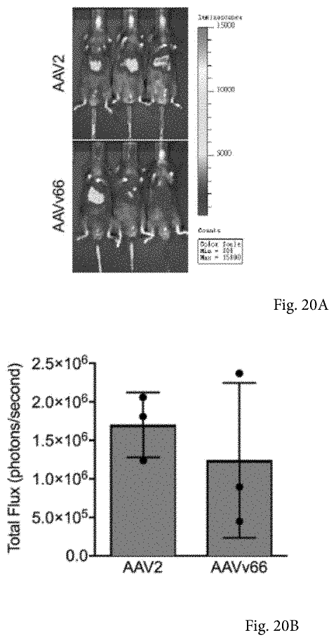

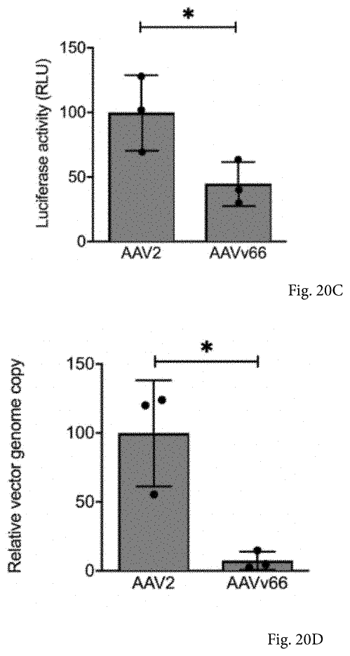

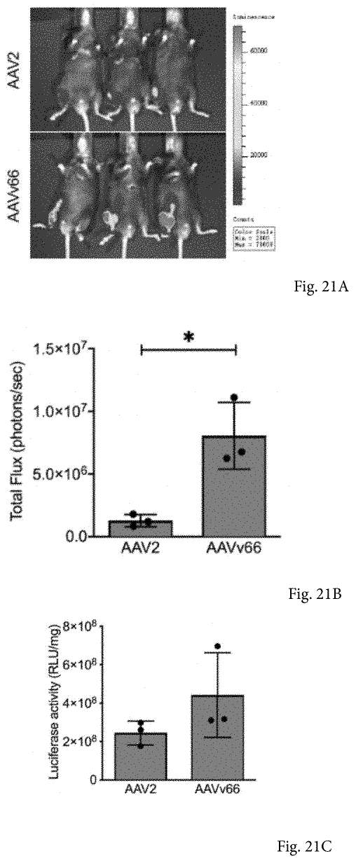

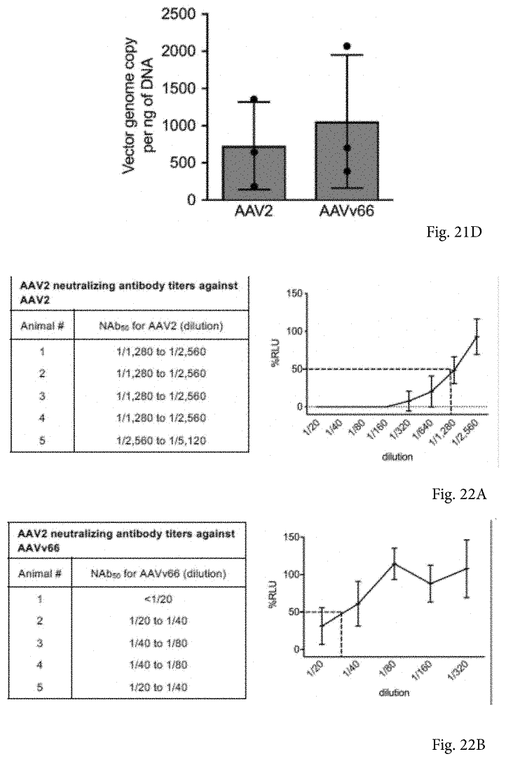

AAVv66 was tested for its capacity to transduce selected target tissues via different routes of administration. To this end, biodistribution of AAVv66 was assessed in mice via multiple routes of delivery (FIGS. 20A-21D). Among all routes tested, the most striking was AAVv66's transduction profile following intracranial delivery to target cells of the central nervous system (CNS) (FIGS. 10A-10D). To determine whether AAVv66 has increased tropism in the CNS, relative to that of AAV2, the Egfp transgene, driven by the ubiquitous chicken-beta actin promoter, was packaged into AAVv66 and AAV2 capsids. Vectors were unilaterally injected into the right hemisphere of the hippocampus at a dose of 3.6E9 vg/animal. Four-weeks post-injection, cryo-sections of treated brains showed that AAVv66 transduced ˜13-fold more cells of the CNS than AAV2, as demonstrated by the enhanced spread throughout the tissue, while AAV2 tended to stay localized to the site of injection (FIGS. 10A-10B). High-magnification imaging of contralateral regions to the site of injection showed that all sub-anatomical regions of the brain (cornu ammonis [CA1, CA2, CA3, and CA4], dentate gyrus, and corpus callosum, FIG. 10C), exhibited detectable levels of EGFP expression (FIG. 10D), indicating that AAVv66 can spread efficiently throughout the hippocampal hemispheres.

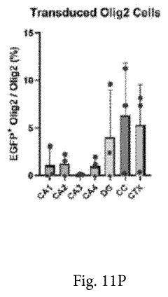

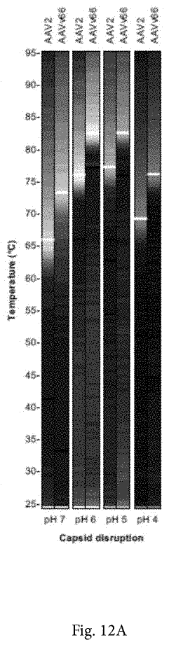

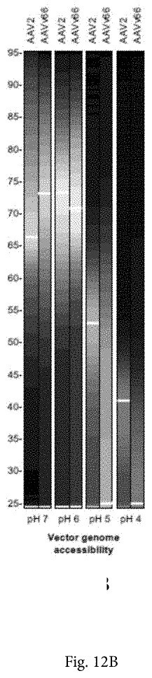

The specific cell types that are transduced by AAVv66 were investigated. Antibody staining was performed with cell type-specific markers; anti-NEUN (neurons), anti-GFAP (astrocytes), anti-IBA1 (microglia), and anti-OLIG2 (oligodendrocytes) (FIGS. 11A, 11E, 11I, and 11M). 3D-volume reconstruction of sub-anatomical CNS regions demonstrates that EGFP expression colocalized with each investigated cell type (FIGS. 11B, 11J, 11F, 11N). Neurons were the predominant cell type found in the cortex and CA1 regions (FIG. 11C). Interestingly, CA2-4 regions and the dentate gyrus exhibited the greatest transduction (˜20-40%). Astrocytes and microglia shared a similar distribution pattern, showing the highest enrichment in the dentate gyrus (FIGS. 11G and 11K). Astrocytes showed approximately 1-7% transduction across all regions (FIG. 11H), while microglia exhibited slightly higher transduction efficiencies (2-12%) (FIG. 11L). Oligodendrocytes were enriched in the corpus callosum (FIG. 110) and had approximately 1-7% transduction by AAVv66 across all regions (FIG. 11P). These data indicate that AAVv66 can transduce all major cell types of the CNS following intrahippocampal injection.

AAVv66 is Serologically Distinct from AAV2

Neutralization of AAV by the host immune system is a major limiting factor for AAV vector transduction efficacy. Individuals harboring pre-existing antibodies against AAV serotypes that are used as capsids in therapeutic vectors are at greater risk to adverse effects and ineffectual treatment. Furthermore, patients requiring repeated administration of an AAV gene therapy, risk poorer transduction efficiencies and stronger immune responses, necessitating alternative vectors.

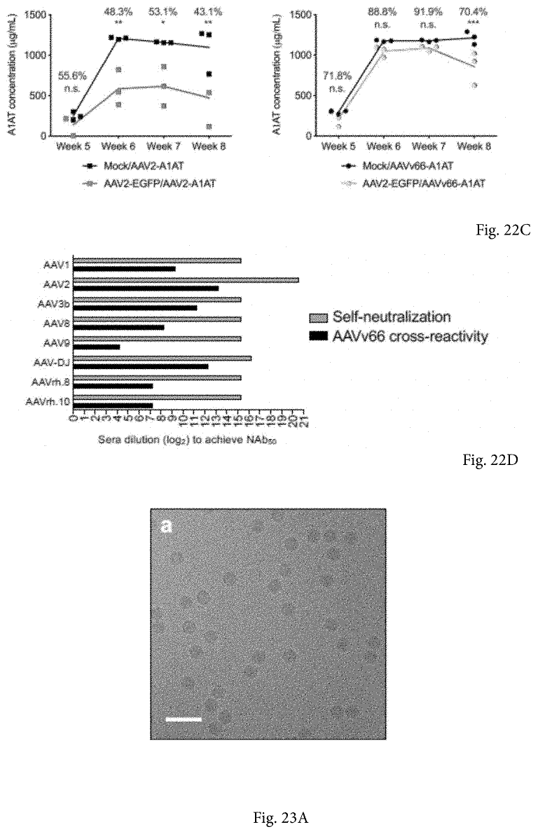

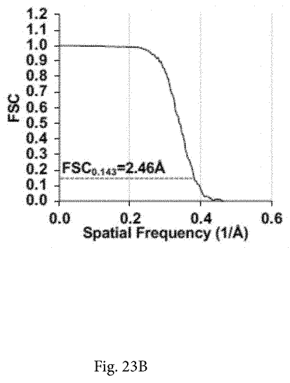

The question of whether AAVv66 transduction can be blocked by pre-immunization with AAV2 was investigated. To create pre-existing anti-AAV2 antibodies in circulation, AAV2-Egfp vectors (1E11 vg/mouse) were intramuscularly delivered to mice. Sera were collected after four weeks to assess neutralizing antibody (NAb) titers in vitro (FIGS. 21A-21D and FIGS. 22A-22B). Low NAb titers were needed to achieve 50% neutralization (NAb50) of AAV2 infection in Huh-7.5 cells (1/1,280˜1/2,560), indicating that antibodies generated from AAV2 pre-immunization are sufficient to inhibit AAV2 transduction. By contrast, the NAb50 for AAVv66 infection with AAV2-treated mouse sera was 1/20˜1/40, indicating that AAVv66 is able to infect cells despite the presence of NAbs generated against AAV2.

To test these findings with a secreted therapeutic transgene product in vivo, we re-dosed AAV2-immunized mice with AAV2 or AAVv66 packaged with the alpha-1 antitrypsin transgene (AAV2-A1AT or AAVv66-A1AT). Sera were collected at weeks 5, 6, 7, and 8, and secreted A1AT levels were quantified by ELISA31 (FIG. 22C). Low A1AT expression would suggest that NAbs generated from the first vector dose were preventing the transduction of the second vector dose. To establish a baseline of “maximal” A1AT expression, naïve mice were also treated in the same fashion. At weeks six and seven, A1AT expression in mice treated with AAV2-Egfp and then AAVv66-A1AT reached ˜90% of A1AT expression as compared to naïve mice, whereas mice re-dosed with AAV2-A1AT reached only ˜40% of naïve levels (FIG. 22C). These results concur with in vitro observations of robust infectivity by AAVv66 in the presence of sera from mice pre-immunized with AAV2 capsids.

Pre-immunity was also tested to investigate whether a broad range of AAV serotypes (AAV1, AAV2, AAV3b, AAV8, AAV9, AAV-DJ, AAVrh.8, and AAVrh.10) can compromise AAVv66 vector transduction. Antisera of rabbits separately pre-immunized with the eight serotypes were screened for AAVv66-vector neutralization. It was observed that, AAV1, AAV3b, and AAV-DJ exhibit about an order of magnitude difference in NAb50 titers compared to AAVv66, whereas AAV2, AAV8, AAVrh.8 and AAVrh.10 exhibit a two-order magnitude difference, and AAV9 had a three-order magnitude difference (FIG. 22D). Taken together, these data indicate that AAVv66 is serologically distinct from AAV2 and some other contemporary AAV capsids.

The AAVv66 Capsid is More Thermostable than AAV2 Across a Range of pHs

The efficient formation and structural stability of the capsid is essential to the production, purification, and storage of viral vectors. Additionally, for productive infection to take place, vector particles must also maintain stability throughout the entry process and uncoat only under conditions in which delivery of the genomic payload can result in transduction of the cell. Although AAV vectors have been studied widely and are utilized for their strong transduction profiles in a range of tissues, the processes of intracellular trafficking, endosomal escape, and transportation of capsids into the nucleus are not fully understood. Among the presumed intracellular checkpoints impacting AAV intracellular trafficking and transduction that are dependent on capsid dynamics, endosomal escape is best understood. This process is believed to be triggered by a pH-dependent structural change of the capsid. Acidification of the endosomal lumen leads to a conformational change of the VP1 domain and exposure of the PLA2 domain within VP1, which triggers escape from the endosome compartment. In principle, vector capsids that can retain stability throughout intracellular trafficking are desirable and may exhibit high transduction capacity.

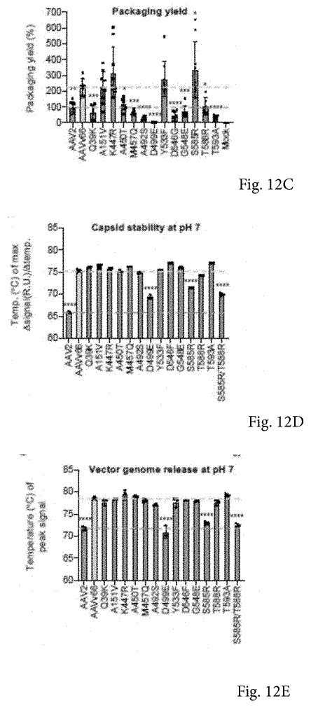

To determine the overall stability of AAVv66 capsids, differential scanning fluorimetry (DSF) analysis was used to measure the thermostability of the AAVv66 capsid across a range of physiological pHs (pH7-pH4) (FIGS. 12A-12E). This range includes pH 4.5, which is observed in the lumen of late endosomes and lysosomes. In this assay, vector particles are suspended in SYPRO Orange dye, which fluoresces upon binding to hydrophobic residues in proteins. Thus, peak fluorescence signals are an indirect readout for maximally bound hydrophobic regions exposed upon protein unfolding. The melting temperatures (maximum slope values [Dsignal/Dtemp], Tm) for AAVv66 are more than five degrees higher than for AAV2 across all pH conditions tested. The most extreme difference was observed at pH 7, where the Tm of AAVv66 (75.29±0.34° C.) is nearly 10 degrees higher than that of AAV2 (65.85±0.18° C.) (FIG. 12A). Thus, the AAVv66 capsid is more thermally stable and resistant to pH than AAV2.

The effect of stability of AAVv66 capsid on vector genome release was investigated. Gauging vector genome release as a function of temperature range has been used as a proxy for pressure-driven DNA extrusion exerted by the nucleolar environment. The temperature dependence of AAVv66 genome release was compared with that of AAV2 under different pHs. To this end, DSF analysis was employed with SYBR Gold dye, which fluoresces upon binding to DNA. Peak fluorescence is an indirect measure of maximal accessibility of the encapsidated genomes to the dye solution. Vector genome release at pH 7 was observed to be concomitant with capsid stability, showing signal peaks at ˜65° C. for AAV2 and ˜74° C. for AAVv66. At lower pHs, however, peak fluorescence for dye-accessible DNA was detected at lower temperatures than peak fluorescence for unfolded capsid protein (FIG. 12B). Furthermore, DNA accessibility for AAVv66 was more evident than that for AAV2—where peak DNA accessibility at pHs 5 and 4 for AAV2 occurred at ˜53° C. and ˜42° C., respectively; and AAVv66 exhibited peak signals at 25° C. This surprising observation shows that in AAVv66 capsids DNA is especially accessible at low pHs (4-5) even at room temperature.

The question of whether AAVv66-specific amino acid residues contribute to the structural and functional differences observed between AAVv66 and AAV2 was then investigated. The thirteen AAVv66-defining amino acid residues were mutated to those of AAV2 and their impact on packaging vector genomes during production with HEK293 cells was tested (FIG. 12C). Additionally, thermal capsid stability and vector genome release were assessed for the mutant capsids (FIGS. 12D and 12E). All but four mutations (A151V, K447R, Y533F, and S585R) resulted in lower yields of DNase-resistant genomes, similar to or lower than those of AAV2 (FIG. 12C). Remarkably, the relatively conservative mutation D499E, which does not involve a charge change, lowered the packaging yield to −5% of AAV2 yields. The modification also affected capsid stability, as D499E along with S585R and the S585R/T588R double mutation lowered Tm by 5.9° C., 3.8° C., and 5.4° C., respectively (FIG. 12D), while other mutations affected the Tm by only 1-2° C. Vector genome accessibilities of the same amino acid mutations exhibit lowered peak signal temperatures, while other mutations led to little or no change (FIG. 12E). Notably, the overall titers of purified vector were not drastically impacted (Table 11). Thus, the packaging yield of AAVv66 is only partially dependent on capsid stability, suggesting that partial capsid destabilization may be sufficient to facilitate genome release. Only residue D499 drastically affects both packaging and capsid stability.

TABLE 11
Virus titer after large-scale production and
purification

| Vector name | Titer GC/mL |
|---|---|
| ssAAV2-CB6-FLuc | 1.60E+12 |
| ssAAVv66-CB6-FLuc | 6.00E+12 |
| ssAAVv66-Q39K-CB6-FLuc | 7.70E+12 |
| ssAAVv66-A151V-CB6-FLuc | 9.00E+12 |
| ssAAVv66-K477R-CB6-FLuc | 6.60E+12 |
| ssAAVv66-A450T-CB6-FLuc | 8.80E+12 |
| ssAAVv66-M457Q-CB6-FLuc | 1.00E+13 |
| ssAAVv66-A492S-CB6-FLuc | 1.40E+13 |
| ssAAVv66-D499E-CB6-FLuc | 9.00E+12 |
| ssAAVv66-Y533F-CB6-FLuc | 7.10E+12 |
| ssAAVv66-D546F-CB6-FLuc | 1.20E+13 |
| ssAAVv66-G548E-CB6-FLuc | 7.80E+12 |
| ssAAVv66-S585R-CB6-FLuc | 7.00E+12 |
| ssAAVv66-T588R-CB6-FLuc | 1.30E+13 |
| ssAAVv66-T593A-CB6-FLuc | 8.10E+12 |
| ssAAVv66-S585R/T588R-CB6-FLuc | 1.85E+12 |

### Cryo-EM Structure Analysis of Capsid Differences Between AAVv66 and AAV2

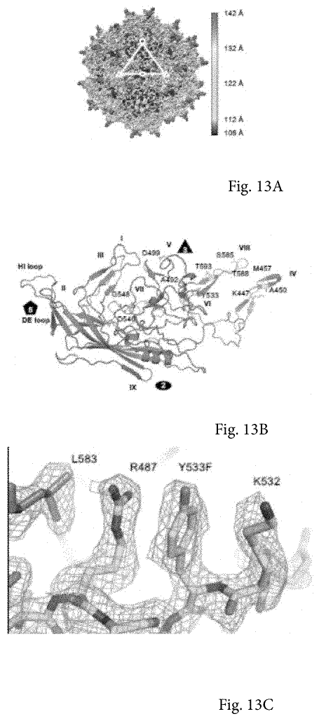

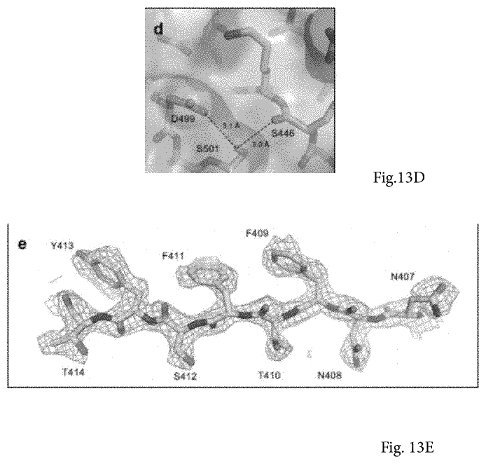

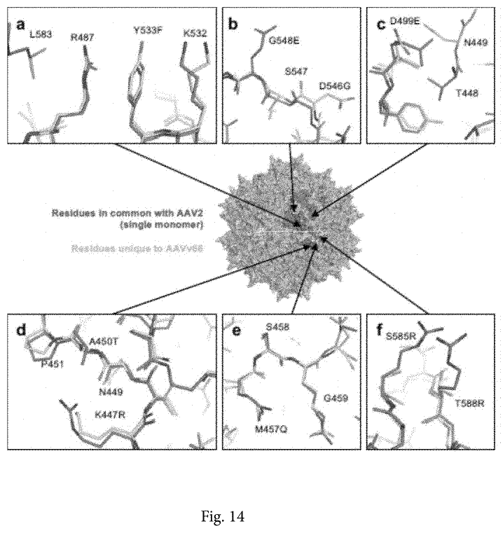

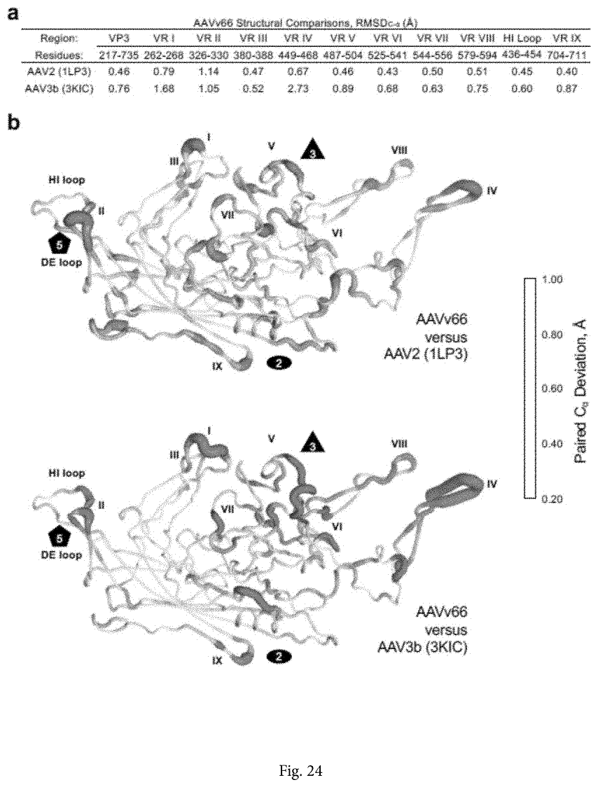

To characterize the structural properties of AAVv66, AAV2v66-Egfp vector was purified for cryo-EM analysis. 52,874 particle images were obtained, which yielded a cryo-EM map at 2.5 Å resolution (FIG. 13E and FIG. 24), and obtained a structural model with optimal real-space fit and stereochemical parameters (Table 9). Overall, the AAVv66 structure is similar to AAV2 (root-mean-square deviation (RMSD) of atomic coordinates=0.456 Å) (FIG. 24). Thus, AAVv66 exhibits the characteristic features of an AAV capsid, which include the depression at two-fold axis, the three-fold symmetry that is defined by the three-fold protrusions, and the five-fold pore that is comprised of five monomers that form the interface and pore for Rep binding (FIG. 13A). Of note, VP1u and VP2 domains are each represented at approximately a twelfth of the VP3 domains for each particle, and similar to other AAV structures before, were not resolved in our symmetrized cryo-EM map. Therefore, only residues 219-736 are definitively resolved within the cryo-EM map, including eleven of thirteen AAVv66-defining residues (FIG. 13B).

Comparison of the AAVv66 structure with that of AAV2 reveals several structural differences, which may contribute to the improved DNA packaging and/or capsid stability. Key differences occur at the interfaces between monomers of VP3 at the protrusions around the 3-fold axis. D499, whose mutation to the longer glutamate residue resulted in dramatic defects in vector genome packaging (FIG. 12C), forms electrostatic interactions and/or hydrogen bonding with S501 (FIG. 13D). This region is tightly packed against the neighboring VP3 monomer (FIG. 13D). Here, the backbone atoms of D499 and 5501 interact with the side chains of the symmetry-related N449 and T448, respectively, whereas the hydroxyl group of S501 hydrogen-bonds with the backbone carbonyl of the symmetry-related S446. The strong effect of D499 mutation is therefore likely due to the disruption of the interface between VP3 monomers, leading to destabilization of the capsid. In the same region, residues K447 and A450 of the neighboring monomer eliminate potential for electrostatic interaction between corresponding AAV2-R447 and T450 side chains (FIG. 14). Amino acid M457 is located on the three-fold protrusion of AAVv66 at variable region IV, with the sidechain poised toward the solvent (FIG. 14). Interestingly, this methionine is a unique feature among other serotypes (FIG. 16), suggesting at potential unique capsid interactions with cellular receptors, host factors, or antibodies. The polar hydroxyl group in AAVv66-Y533 (AAV2-F533) likely stabilizes the polar environment between the side chains of R487 and K532 and may contribute to the interaction with L583 of the symmetry-related monomer (FIG. 14). AAVv66-D546 and G548 redistribute the surface charge conferred by AAV2-G546 and E548 (FIG. 14) and is yet another defining feature of AAVv66.

A key functional region of AAV2 involves the positively charged arginine residues at position 585 and 588. These residues are at the surface of the three-fold protrusions and govern the capsid's interaction with HSPG receptors, which are vital to attachment and entry in many cell types. By contrast, S585 and T588 in the AAVv66 capsid are neutral charged polar residues (FIG. 13D and FIG. 14), similar to S586 and T589 of AAV3b (FIG. 16). AAV3b's physical and functional interactions with HSPG rely on electrostatic interactions conferred by residues R447 and R594 (R447 and A593 in AAV2)7, but AAVv66 also lacks these arginine residues (K447 and T593). These differences from AAV2 and AAV3b suggest that AAVv66 associates differently with the canonical cell surface receptor commonly utilized by AAV clade B and C capsids, consistent with our findings that AAVv66 lacks heparin binding.

### AAV2 and AAVv66 Show Surface Charge Differences

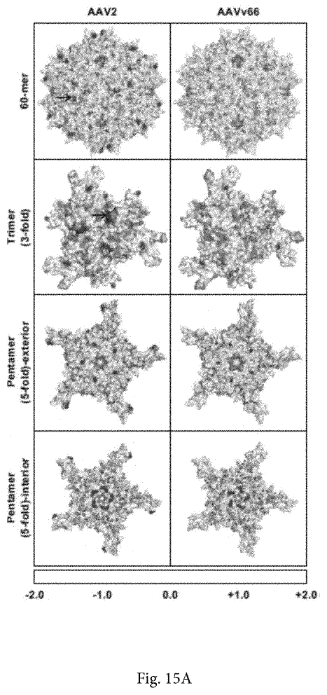

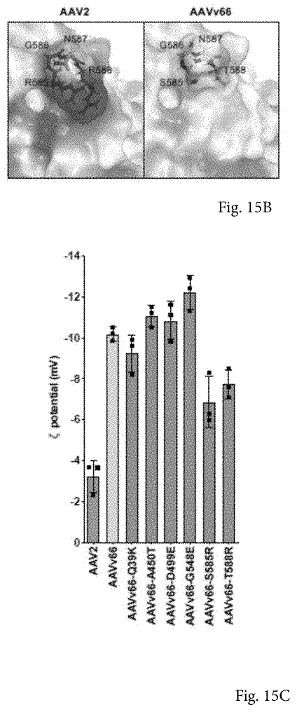

Because electrostatic properties of the virus are important for capsid-receptor interaction 7,43, how the net loss of positive charge for the AAVv66 capsid in relation to AAV2 affects the electrostatic properties of the capsid was investigated. First, the calculated electrostatic potential values for AAV2 and AAVv66 structures were compared (FIG. 15A). The distribution of electrostatic potential on the surface of AAVv66 differs from that of AAV2. The most notable difference is at the three-fold protrusions, where the positive charge conferred by R585 and R588 in AAV2 is drastically reduced by S585 and T588 in AAVv66 (FIG. 15B).

Whether the distinct structure and surface electrostatics of AAVv66 affects the charge-dependent particle migration (zeta potential) of the capsid was then investigated (FIG. 15C). The zeta potential of AAVv66 (−10 mV) is remarkably different from that of AAV2 (−3.5 mV), consistent with differences in electrostatic potential between the capsids. To test the contributions of individual substitutions, particle migration of AAVv66 harboring single amino acid substitutions that convert residues to the corresponding residues of AAV2 was measured (FIG. 15C). Single mutations S585R and T588R resulted in the most dramatic change of the zeta potential (by ˜3 mV each), bringing the zeta potential closer to that of AAV2 (FIG. 15C). These observations indicate that the electrostatic properties of AAVv66 differ from those of AAV2 and the difference is predominantly due to substitutions at positions 585 and 588. Thus, interactions of capsid AAVv66 with receptors, antibodies and other proteins likely differ substantially from those of other closely related capsids.

This disclosure is not limited in its application to the details of construction and the arrangement of components set forth in this description or illustrated in the drawings. The disclosure is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Having thus described several aspects of at least one embodiment of this disclosure, it is to be appreciated various alterations, modifications, and improvements will readily occur to those skilled in the art. Such alterations, modifications, and improvements are intended to be part of this disclosure, and are intended to be within the spirit and scope of the disclosure. Accordingly, the foregoing description and drawings are by way of example only.

What is claimed is:

1. A method for delivering a transgene to a target cell in a subject, the method comprising intracranially administering to the subject a recombinant adeno-associated virus (rAAV) comprising:
(i) an isolated nucleic acid comprising a transgene encoding one or more gene products of interest; and
(ii) an adeno-associated acid (AAV) capsid protein having the sequence set forth in SEQ ID NO: 66.

2. The method of claim 1, wherein the intracranial administration comprises intrahippocampal injection.

3. The method of claim 1, wherein the target cell is a central nervous system (CNS) cell.

4. The method of claim 3 wherein the CNS cell is a neuron, oligodendrocyte, astrocyte, or microglial cell.

5. The method of claim 1, wherein the subject is a mammal, optionally wherein the mammal is a human.

6. The method of claim 1, wherein the subject is characterized by production of anti-AAV2 antibodies.

7. The method of claim 6, wherein after administration of the rAAV, the subject does not elicit a neutralizing immune response against the rAAV.

8. The method of claim 1, wherein the isolated nucleic acid comprises AAV inverted terminal repeats (ITRs) flanking the transgene.

9. The method of claim 1, wherein the nucleic acid sequence encoding the one or more gene products is operably linked to a promoter.

10. The method of claim 1, wherein the one or more gene products comprise a protein or an inhibitory nucleic acid.

11. A method for delivering a transgene to a target cell in a subject, the method comprising intravenously administering to the subject a recombinant adeno-associated virus (rAAV) comprising:
(i) an isolated nucleic acid comprising a transgene encoding one or more gene products of interest; and
(ii) an adeno-associated acid (AAV) capsid protein having the sequence set forth in SEQ ID NO: 66,
wherein the administration results in the rAAV crossing the blood brain barrier (BBB) of the subject.

12. The method of claim 11, wherein the target cell is a central nervous system (CNS) cell.

13. The method of claim 12 wherein the CNS cell is a neuron, oligodendrocyte, astrocyte, or microglial cell.

14. The method of claim 11, wherein the administration results in decreased transduction of liver cells relative to administration of an rAAV having an AAV2 capsid protein

15. The method of claim 11, wherein the subject is a mammal, optionally wherein the mammal is a human.

16. The method of claim 11, wherein the subject is characterized by production of anti-AAV2 antibodies.

17. The method of claim 16, wherein after administration of the rAAV, the subject does not elicit a neutralizing immune response against the rAAV.

18. The method of claim 11, wherein the isolated nucleic acid comprises AAV inverted terminal repeats (ITRs) flanking the transgene.

19. The method of claim 11, wherein the nucleic acid sequence encoding the one or more gene products is operably linked to a promoter.

20. The method of claim 11, wherein the one or more gene products comprise a protein or an inhibitory nucleic acid.
